SEQUENCE LISTING (304 sequences)

SEQ ID NO: 1 - DNA, 24 bp, Artificial Sequence
  synthetic oligonucleotide
cggcatcattacgtcaagcaaaag

SEQ ID NO: 2 - DNA, 24 bp, Artificial Sequence
  synthetic oligonucleotide
gcgccagcgccgcgagggactcag

SEQ ID NO: 3 - DNA, 44 bp, Artificial Sequence
  synthetic oligonucleotide
gaacttcggaataggaactaatagtaaaccctgttttccttgcc

SEQ ID NO: 4 - DNA, 44 bp, Artificial Sequence
  synthetic oligonucleotide
gaagcagctccagcctacaccatcatttgctcccaaaatctttc

SEQ ID NO: 5 - DNA, 30 bp, Artificial Sequence
  synthetic oligonucleotide
cccgaattccgaagtgcttttaatgtgtcg

SEQ ID NO: 6 - DNA, 21 bp, Artificial Sequence
  synthetic oligonucleotide
cgcctggatctgtcatcggtg

SEQ ID NO: 7 - DNA, 23 bp, Artificial Sequence
  synthetic oligonucleotide
cgtgagttgctactactcaatag

SEQ ID NO: 8 - DNA, 20 bp, Artificial Sequence
  synthetic oligonucleotide
gccggacgaatcagataaag

SEQ ID NO: 9 - DNA, 42 bp, Artificial Sequence
  synthetic oligonucleotide
gaacttcggaataggaactaaggtttaaaaccttgcgtggtc

SEQ ID NO: 10 - DNA, 43 bp, Artificial Sequence
  synthetic oligonucleotide
gaagcagctccagcctacacgaaattacgtcatcagacgtcgc

SEQ ID NO: 11 - DNA, 20 bp, Artificial Sequence
  synthetic oligonucleotide
gattgattcataaatactcc

SEQ ID NO: 12 - DNA, 22 bp, Artificial Sequence
  synthetic oligonucleotide
caaacagtaagttaatgaaatc

SEQ ID NO: 13 - DNA, 20 bp, Artificial Sequence
  synthetic oligonucleotide
cactattgcatcagaagcgg

SEQ ID NO: 14 - DNA, 21 bp, Artificial Sequence
  synthetic oligonucleotide
cctttctggttcgaaaagtgg

SEQ ID NO: 15 - DNA, 50 bp, Artificial Sequence
  synthetic oligonucleotide
cttcacgaggcagacctcagcgcctgatttttaccgttgcatcatgtcgc

SEQ ID NO: 16 - DNA, 50 bp, Artificial Sequence
  synthetic oligonucleotide
gattttgagacacaacgtggctttcatttttactccttaattacgccgac

SEQ ID NO: 17 - DNA, 20 bp, Artificial Sequence
  synthetic oligonucleotide
ggaataccttacattgatga

SEQ ID NO: 18 - DNA, 21 bp, Artificial Sequence
  synthetic oligonucleotide
ctttagatgcctttaatttag

SEQ ID NO: 19 - DNA, 20 bp, Artificial Sequence
  synthetic oligonucleotide
ccatgctgatgattgcagcc

SEQ ID NO: 20 - DNA, 20 bp, Artificial Sequence
  synthetic oligonucleotide
ccgacgccacaaccagtgac

SEQ ID NO: 21 - DNA, 47 bp, Artificial Sequence
  synthetic oligonucleotide
cttcacgaggcagacctcagcgcctaatgagtcattctaccgatcac

SEQ ID NO: 22 - DNA, 50 bp, Artificial Sequence
  synthetic oligonucleotide
gattttgagacacaacgtggctttcatttttcagccctgccttagtaatc

SEQ ID NO: 23 - DNA, 20 bp, Artificial Sequence
  synthetic oligonucleotide
cagggatgacctactggtgg

SEQ ID NO: 24 - DNA, 20 bp, Artificial Sequence
  synthetic oligonucleotide
ggattcgcgccaaaccacag

SEQ ID NO: 25 - DNA, 20 bp, Artificial Sequence
  synthetic oligonucleotide
gtttaaccgcaaccatatgc

SEQ ID NO: 26 - DNA, 20 bp, Artificial Sequence
  synthetic oligonucleotide
cgattcctgcttctaatatc

SEQ ID NO: 27 - DNA, 48 bp, Artificial Sequence
  synthetic oligonucleotide
cttcacgaggcagacctcagcgcctaatatgcagtgattttttttgcc

SEQ ID NO: 28 - DNA, 44 bp, Artificial Sequence
  synthetic oligonucleotide
gattttgagacacaacgtggctttcataatagccccctgttgaa

SEQ ID NO: 29 - DNA, 20 bp, Artificial Sequence
  synthetic oligonucleotide
cctgactgcgctgaaagtcg

SEQ ID NO: 30 - DNA, 20 bp, Artificial Sequence
  synthetic oligonucleotide
gacgccgatactcgtttacc

SEQ ID NO: 31 - DNA, 20 bp, Artificial Sequence
  synthetic oligonucleotide
cgccgcgttgcagaatgttg

SEQ ID NO: 32 - DNA, 28 bp, Artificial Sequence
  synthetic oligonucleotide
ccggatgtggcgtatgctgataagacgc

SEQ ID NO: 33 - DNA, 50 bp, Artificial Sequence
  synthetic oligonucleotide
cttcacgaggcagacctcagcgcccaacgcggtagttcgctaaactgccg

SEQ ID NO: 34 - DNA, 44 bp, Artificial Sequence
  synthetic oligonucleotide
gattttgagacacaacgtggctttccatttttttgctttccttc

SEQ ID NO: 35 - DNA, 20 bp, Artificial Sequence
  synthetic oligonucleotide
atttttgaaattaacgctcg

SEQ ID NO: 36 - DNA, 20 bp, Artificial Sequence
  synthetic oligonucleotide
gttgaaaccgcaacccgttc

SEQ ID NO: 37 - DNA, 20 bp, Artificial Sequence
  synthetic oligonucleotide
gatcacttccacatcttcag

SEQ ID NO: 38 - DNA, 20 bp, Artificial Sequence
  synthetic oligonucleotide
gatttcatttttaaatgcgg

SEQ ID NO: 39 - DNA, 49 bp, Artificial Sequence
  synthetic oligonucleotide
cttcacgaggcagacctcagcgcctaaggagtcacaatggaacaggttg

SEQ ID NO: 40 - DNA, 44 bp, Artificial Sequence
  synthetic oligonucleotide
gattttgagacacaacgtggctttcatgtcagtctcctgaatcc

SEQ ID NO: 41 - DNA, 20 bp, Artificial Sequence
  synthetic oligonucleotide
ctggcctcaatacccagttg

SEQ ID NO: 42 - DNA, 25 bp, Artificial Sequence
  synthetic oligonucleotide
gtttactggatcaaacgccggacgc

SEQ ID NO: 43 - DNA, 20 bp, Artificial Sequence
  synthetic oligonucleotide
gtctgcaaacgctcaacgac

SEQ ID NO: 44 - DNA, 20 bp, Artificial Sequence
  synthetic oligonucleotide
gtcgttctccagatcttccg

SEQ ID NO: 45 - DNA, 44 bp, Artificial Sequence
  synthetic oligonucleotide
cttcacgaggcagacctcagcgcctaataccgtcctttctacag

SEQ ID NO: 46 - DNA, 48 bp, Artificial Sequence
  synthetic oligonucleotide
gattttgagacacaacgtggctttccatcggtctcgctccagttaatc

SEQ ID NO: 47 - DNA, 20 bp, Artificial Sequence
  synthetic oligonucleotide
gccgctgtttgacggtggac

SEQ ID NO: 48 - DNA, 20 bp, Artificial Sequence
  synthetic oligonucleotide
cgcccagtactcggaataac

SEQ ID NO: 49 - DNA, 20 bp, Artificial Sequence
  synthetic oligonucleotide
cctttgaaccgttggggctg

SEQ ID NO: 50 - DNA, 20 bp, Artificial Sequence
  synthetic oligonucleotide
cttatctactcgaatttggc

SEQ ID NO: 51 - DNA, 46 bp, Artificial Sequence
  synthetic oligonucleotide
cttcacgaggcagacctcagcgccgataacataacgttgtaaaaac

SEQ ID NO: 52 - DNA, 50 bp, Artificial Sequence
  synthetic oligonucleotide
gattttgagacacaacgtggctttcatttcgaactccttaaatttatttg

SEQ ID NO: 53 - DNA, 20 bp, Artificial Sequence
  synthetic oligonucleotide
gttgcgcggagatgaaaatg

SEQ ID NO: 54 - DNA, 20 bp, Artificial Sequence
  synthetic oligonucleotide
catgaagatttaatgcctcc

SEQ ID NO: 55 - DNA, 20 bp, Artificial Sequence
  synthetic oligonucleotide
gcactgctctcgattgtctg

SEQ ID NO: 56 - DNA, 20 bp, Artificial Sequence
  synthetic oligonucleotide
gggccgctcatacctgaatg

SEQ ID NO: 57 - DNA, 47 bp, Artificial Sequence
  synthetic oligonucleotide
cttcacgaggcagacctcagcgcctgattatttccgcagccagcgat

SEQ ID NO: 58 - DNA, 49 bp, Artificial Sequence
  synthetic oligonucleotide
gattttgagacacaacgtggctttccattaaggaatcatccacgttaag

SEQ ID NO: 59 - DNA, 20 bp, Artificial Sequence
  synthetic oligonucleotide
ggtggcgcgctgtatgagtg

SEQ ID NO: 60 - DNA, 20 bp, Artificial Sequence
  synthetic oligonucleotide
cctaaatcatgtggacgacc

SEQ ID NO: 61 - DNA, 26 bp, Artificial Sequence
  synthetic oligonucleotide
acgactccagcggatcgcgcggcaac

SEQ ID NO: 62 - DNA, 24 bp, Artificial Sequence
  synthetic oligonucleotide
caatagtggaattgttgctttatc

SEQ ID NO: 63 - DNA, 49 bp, Artificial Sequence
  synthetic oligonucleotide
cttcacgaggcagacctcagcgcctagcccttgtggaggttcctgcaat

SEQ ID NO: 64 - DNA, 46 bp, Artificial Sequence
  synthetic oligonucleotide
gattttgagacacaacgtggctttcatttctcatccttgttttatc

SEQ ID NO: 65 - DNA, 23 bp, Artificial Sequence
  synthetic oligonucleotide
ggttttcgaccagtttgactaag

SEQ ID NO: 66 - DNA, 24 bp, Artificial Sequence
  synthetic oligonucleotide
cgttggtgctgttagcactgtatc

SEQ ID NO: 67 - DNA, 23 bp, Artificial Sequence
  synthetic oligonucleotide
gcataagtcacccgatatggtgg

SEQ ID NO: 68 - DNA, 21 bp, Artificial Sequence
  synthetic oligonucleotide
ctcgctgggcgaactgatggg

SEQ ID NO: 69 - DNA, 50 bp, Artificial Sequence
  synthetic oligonucleotide
cttcacgaggcagacctcagcgcctaattgattagcggataataaaaaac

SEQ ID NO: 70 - DNA, 48 bp, Artificial Sequence
  synthetic oligonucleotide
gattttgagacacaacgtggctttcatcctaaactccttattatattg

SEQ ID NO: 71 - DNA, 21 bp, Artificial Sequence
  synthetic oligonucleotide
ctgcaacgcggcgacgatttc

SEQ ID NO: 72 - DNA, 21 bp, Artificial Sequence
  synthetic oligonucleotide
ggcaaaacttcctccaaaccg

SEQ ID NO: 73 - DNA, 20 bp, Artificial Sequence
  synthetic oligonucleotide
cctttcttatctggaacaac

SEQ ID NO: 74 - DNA, 20 bp, Artificial Sequence
  synthetic oligonucleotide
cgcccagacgcaggccaaac

SEQ ID NO: 75 - DNA, 43 bp, Artificial Sequence
  synthetic oligonucleotide
cttcacgaggcagacctcagcgcctaatcggtatcggaatcag

SEQ ID NO: 76 - DNA, 51 bp, Artificial Sequence
  synthetic oligonucleotide
gattttgagacacaacgtggctttcattgctctataaactccgtacatcac

SEQ ID NO: 77 - DNA, 20 bp, Artificial Sequence
  synthetic oligonucleotide
catgagactgacatctaaag

SEQ ID NO: 78 - DNA, 20 bp, Artificial Sequence
  synthetic oligonucleotide
cttcttttacgaagtccagc

SEQ ID NO: 79 - DNA, 20 bp, Artificial Sequence
  synthetic oligonucleotide
catcgcaaaagaagagatgg

SEQ ID NO: 80 - DNA, 20 bp, Artificial Sequence
  synthetic oligonucleotide
ctcagcgcctgggtatcgag

SEQ ID NO: 81 - DNA, 46 bp, Artificial Sequence
  synthetic oligonucleotide
cttcacgaggcagacctcagcgcctaagagttgaggtttggttatg

SEQ ID NO: 82 - DNA, 45 bp, Artificial Sequence
  synthetic oligonucleotide
gattttgagacacaacgtggctttcattataaattctcctgattc

SEQ ID NO: 83 - DNA, 20 bp, Artificial Sequence
  synthetic oligonucleotide
ggtgcgctgtatgtacgtcg

SEQ ID NO: 84 - DNA, 20 bp, Artificial Sequence
  synthetic oligonucleotide
ggttaatggttgcagattgc

SEQ ID NO: 85 - DNA, 20 bp, Artificial Sequence
  synthetic oligonucleotide
acatgttgctattcccgatg

SEQ ID NO: 86 - DNA, 20 bp, Artificial Sequence
  synthetic oligonucleotide
gcagtgtgggcgaaggagac

SEQ ID NO: 87 - DNA, 46 bp, Artificial Sequence
  synthetic oligonucleotide
cacgaggcagacctcagcgctaacccgtcttgagacagaaacaaac

SEQ ID NO: 88 - DNA, 45 bp, Artificial Sequence
  synthetic oligonucleotide
ttgagacacaacgtggctttcatccattccttctttttattcccg

SEQ ID NO: 89 - DNA, 20 bp, Artificial Sequence
  synthetic oligonucleotide
atctttccgtctggtatctg

SEQ ID NO: 90 - DNA, 20 bp, Artificial Sequence
  synthetic oligonucleotide
gactcgccagatgtgctcac

SEQ ID NO: 91 - DNA, 32 bp, Artificial Sequence
  synthetic oligonucleotide
cccgagctcaccatcatcggtgccgtagcgag

SEQ ID NO: 92 - DNA, 25 bp, Artificial Sequence
  synthetic oligonucleotide
gataatcaattaccgaaaagccatc

SEQ ID NO: 93 - DNA, 46 bp, Artificial Sequence
  synthetic oligonucleotide
cttcacgaggcagacctcagcgcctaaaagaattcaccatgagcgg

SEQ ID NO: 94 - DNA, 50 bp, Artificial Sequence
  synthetic oligonucleotide
gattttgagacacaacgtggctttccataatgttgtcctcttgatttctg

SEQ ID NO: 95 - DNA, 23 bp, Artificial Sequence
  synthetic oligonucleotide
cagttcaccgtactcaatcacgc

SEQ ID NO: 96 - DNA, 26 bp, Artificial Sequence
  synthetic oligonucleotide
cgagttgctgaatatcctgccactcc

SEQ ID NO: 97 - DNA, 20 bp, Artificial Sequence
  synthetic oligonucleotide
ggtattggtcgtccgctggg

SEQ ID NO: 98 - DNA, 20 bp, Artificial Sequence
  synthetic oligonucleotide
cgctcacgttgcgcttccac

SEQ ID NO: 99 - DNA, 45 bp, Artificial Sequence
  synthetic oligonucleotide
cttcacgaggcagacctcagcgcctagttttaatcaggttgtatg

SEQ ID NO: 100 - DNA, 45 bp, Artificial Sequence
  synthetic oligonucleotide
gattttgagacacaacgtggctttcatattcaagatgtcctgtag

SEQ ID NO: 101 - DNA, 20 bp, Artificial Sequence
  synthetic oligonucleotide
ggcgtgaccatggtttatac

SEQ ID NO: 102 - DNA, 20 bp, Artificial Sequence
  synthetic oligonucleotide
gaagttcgcgagccggagcc

SEQ ID NO: 103 - DNA, 27 bp, Artificial Sequence
  synthetic oligonucleotide
gacctgatgctggtcagccaggcgtag

SEQ ID NO: 104 - DNA, 21 bp, Artificial Sequence
  synthetic oligonucleotide
cgcttcagaaggtactcccag

SEQ ID NO: 105 - DNA, 44 bp, Artificial Sequence
  synthetic oligonucleotide
cttcacgaggcagacctcagcgcccaggcgttgacgatagcggg

SEQ ID NO: 106 - DNA, 46 bp, Artificial Sequence
  synthetic oligonucleotide
gattttgagacacaacgtggctttcatccgaggtaaagtctcccca

SEQ ID NO: 107 - DNA, 25 bp, Artificial Sequence
  synthetic oligonucleotide
gaaccttccgttaccgggcctttac

SEQ ID NO: 108 - DNA, 24 bp, Artificial Sequence
  synthetic oligonucleotide
gcaacattgcattaatgcgacgac

SEQ ID NO: 109 - DNA, 20 bp, Artificial Sequence
  synthetic oligonucleotide
gcataaagtggcgagtctgg

SEQ ID NO: 110 - DNA, 20 bp, Artificial Sequence
  synthetic oligonucleotide
gtaatttcgattcggtgtcc

SEQ ID NO: 111 - DNA, 44 bp, Artificial Sequence
  synthetic oligonucleotide
gaacttcggaataggaactaagtttgagcaggcgcaaacccagc

SEQ ID NO: 112 - DNA, 40 bp, Artificial Sequence
  synthetic oligonucleotide
gaagcagctccagcctacaccatgcctaatacccttatcc

SEQ ID NO: 113 - DNA, 20 bp, Artificial Sequence
  synthetic oligonucleotide
ggtcctggttgaacgggtcc

SEQ ID NO: 114 - DNA, 20 bp, Artificial Sequence
  synthetic oligonucleotide
gttccagcgtttcaccatcg

SEQ ID NO: 115 - DNA, 20 bp, Artificial Sequence
  synthetic oligonucleotide
ccatttcgatatctcttcac

SEQ ID NO: 116 - DNA, 20 bp, Artificial Sequence
  synthetic oligonucleotide
cgtcctcgcatttgttatgc

SEQ ID NO: 117 - DNA, 44 bp, Artificial Sequence
  synthetic oligonucleotide
cttcacgaggcagacctcagcgccatgatcaaagagtgggctac

SEQ ID NO: 118 - DNA, 44 bp, Artificial Sequence
  synthetic oligonucleotide
gattttgagacacaacgtggctttcattactgcgattgcgttcc

SEQ ID NO: 119 - DNA, 20 bp, Artificial Sequence
  synthetic oligonucleotide
cttaatccgtgactcaatgc

SEQ ID NO: 120 - DNA, 20 bp, Artificial Sequence
  synthetic oligonucleotide
gaaatgttcataccgtatgg

SEQ ID NO: 121 - DNA, 20 bp, Artificial Sequence
  synthetic oligonucleotide
cgcaccgcaggttgctgaac

SEQ ID NO: 122 - DNA, 24 bp, Artificial Sequence
  synthetic oligonucleotide
gtgatgttttcactcccctgattc

SEQ ID NO: 123 - DNA, 44 bp, Artificial Sequence
  synthetic oligonucleotide
cttcacgaggcagacctcagcgcctgatgagttccaaattatgc

SEQ ID NO: 124 - DNA, 50 bp, Artificial Sequence
  synthetic oligonucleotide
gattttgagacacaacgtggctttcatattgcaccgcttttgttaaccag

SEQ ID NO: 125 - DNA, 20 bp, Artificial Sequence
  synthetic oligonucleotide
gctgattggcacacaagggc

SEQ ID NO: 126 - DNA, 24 bp, Artificial Sequence
  synthetic oligonucleotide
cattgattcagtcaatagccaatg

SEQ ID NO: 127 - DNA, 22 bp, Artificial Sequence
  synthetic oligonucleotide
gcaatcacgtctgccgtttacc

SEQ ID NO: 128 - DNA, 21 bp, Artificial Sequence
  synthetic oligonucleotide
gatcggatgctcgtaaaagcc

SEQ ID NO: 129 - DNA, 46 bp, Artificial Sequence
  synthetic oligonucleotide
cttcacgaggcagacctcagcgccccgatcaacctaaaccgctggg

SEQ ID NO: 130 - DNA, 44 bp, Artificial Sequence
  synthetic oligonucleotide
gattttgagacacaacgtggctttcataggacctccgttcattg

SEQ ID NO: 131 - DNA, 22 bp, Artificial Sequence
  synthetic oligonucleotide
cgttcaaacatctgcatcagag

SEQ ID NO: 132 - DNA, 22 bp, Artificial Sequence
  synthetic oligonucleotide
ggcgtcgcgttggcgtggttag

SEQ ID NO: 133 - DNA, 20 bp, Artificial Sequence
  synthetic oligonucleotide
gacacgctgaattttatgcc

SEQ ID NO: 134 - DNA, 20 bp, Artificial Sequence
  synthetic oligonucleotide
ctgccctgccgtcagtttcg

SEQ ID NO: 135 - DNA, 51 bp, Artificial Sequence
  synthetic oligonucleotide
cttcacgaggcagacctcagcgcctaatctttcagccaaaaaacttaagac

SEQ ID NO: 136 - DNA, 47 bp, Artificial Sequence
  synthetic oligonucleotide
gattttgagacacaacgtggctttccatattctatatccttcctttc

SEQ ID NO: 137 - DNA, 20 bp, Artificial Sequence
  synthetic oligonucleotide
gtcggtatcgtgagaacacc

SEQ ID NO: 138 - DNA, 20 bp, Artificial Sequence
  synthetic oligonucleotide
cttacagacatccaggcgtg

SEQ ID NO: 139 - DNA, 20 bp, Artificial Sequence
  synthetic oligonucleotide
ggcttgttagcggcatatcg

SEQ ID NO: 140 - DNA, 20 bp, Artificial Sequence
  synthetic oligonucleotide
gacacgtttttcactacgtg

SEQ ID NO: 141 - DNA, 44 bp, Artificial Sequence
  synthetic oligonucleotide
cttcacgaggcagacctcagcgcctaaataaaggatacacaatg

SEQ ID NO: 142 - DNA, 45 bp, Artificial Sequence
  synthetic oligonucleotide
gattttgagacacaacgtggctttcatcgtgatcccttaagcatc

SEQ ID NO: 143 - DNA, 20 bp, Artificial Sequence
  synthetic oligonucleotide
cgcgagcatttacagatgcc

SEQ ID NO: 144 - DNA, 20 bp, Artificial Sequence
  synthetic oligonucleotide
gcttcaccgtactgcttacg

SEQ ID NO: 145 - DNA, 20 bp, Artificial Sequence
  synthetic oligonucleotide
cagcttcagttgatttcgcc

SEQ ID NO: 146 - DNA, 20 bp, Artificial Sequence
  synthetic oligonucleotide
cagttggttttcatgggtcg

SEQ ID NO: 147 - DNA, 48 bp, Artificial Sequence
  synthetic oligonucleotide
cttcacgaggcagacctcagcgcctaagtaaatttaggattaatcctg

SEQ ID NO: 148 - DNA, 45 bp, Artificial Sequence
  synthetic oligonucleotide
gattttgagacacaacgtggctttccataatcaatccagcccctg

SEQ ID NO: 149 - DNA, 20 bp, Artificial Sequence
  synthetic oligonucleotide
gcaaatctttcagtctttcc

SEQ ID NO: 150 - DNA, 20 bp, Artificial Sequence
  synthetic oligonucleotide
catttcaaagccaacatacg

SEQ ID NO: 151 - DNA, 20 bp, Artificial Sequence
  synthetic oligonucleotide
gtctgatgacctgttatgac

SEQ ID NO: 152 - DNA, 20 bp, Artificial Sequence
  synthetic oligonucleotide
caacgccaccagatgtgttc

SEQ ID NO: 153 - DNA, 44 bp, Artificial Sequence
  synthetic oligonucleotide
cttcacgaggcagacctcagcgcctgacgtggcgagcaggacgc

SEQ ID NO: 154 - DNA, 42 bp, Artificial Sequence
  synthetic oligonucleotide
gattttgagacacaacgtggctttcatagataccttcttgac

SEQ ID NO: 155 - DNA, 20 bp, Artificial Sequence
  synthetic oligonucleotide
tgatgctggtaatgaaatcg

SEQ ID NO: 156 - DNA, 20 bp, Artificial Sequence
  synthetic oligonucleotide
cgcggtcgtatggatcgtgc

SEQ ID NO: 157 - DNA, 20 bp, Artificial Sequence
  synthetic oligonucleotide
tacttttaaaggccgtgaag

SEQ ID NO: 158 - DNA, 20 bp, Artificial Sequence
  synthetic oligonucleotide
gcccgaggatgcgcttctat

SEQ ID NO: 159 - DNA, 43 bp, Artificial Sequence
  synthetic oligonucleotide
cttcacgaggcagacctcagcgcctaactcgcttctccgttac

SEQ ID NO: 160 - DNA, 44 bp, Artificial Sequence
  synthetic oligonucleotide
gattttgagacacaacgtggctttcatgtcagctccggcgtaac

SEQ ID NO: 161 - DNA, 20 bp, Artificial Sequence
  synthetic oligonucleotide
cggacacactgacaaagcag

SEQ ID NO: 162 - DNA, 20 bp, Artificial Sequence
  synthetic oligonucleotide
ccatattgacgtttaatgcc

SEQ ID NO: 163 - DNA, 20 bp, Artificial Sequence
  synthetic oligonucleotide
tcatggaagacgaaacgttg

SEQ ID NO: 164 - DNA, 20 bp, Artificial Sequence
  synthetic oligonucleotide
cggaagtgaaaactgtctct

SEQ ID NO: 165 - DNA, 41 bp, Artificial Sequence
  synthetic oligonucleotide
cttcacgaggcagacctcagcgcctaaaaaagccgcatgtg

SEQ ID NO: 166 - DNA, 44 bp, Artificial Sequence
  synthetic oligonucleotide
gattttgagacacaacgtggctttcatacattgaccttcacatc

SEQ ID NO: 167 - DNA, 20 bp, Artificial Sequence
  synthetic oligonucleotide
caacctggctacataatgcc

SEQ ID NO: 168 - DNA, 20 bp, Artificial Sequence
  synthetic oligonucleotide
gatacctacaaaacgtttgc

SEQ ID NO: 169 - DNA, 20 bp, Artificial Sequence
  synthetic oligonucleotide
cgggcggtggggatgtttag

SEQ ID NO: 170 - DNA, 20 bp, Artificial Sequence
  synthetic oligonucleotide
cagtggttaaagagtggcgg

SEQ ID NO: 171 - DNA, 43 bp, Artificial Sequence
  synthetic oligonucleotide
cttcacgaggcagacctcagcgcctaatctcacccgctaacac

SEQ ID NO: 172 - DNA, 43 bp, Artificial Sequence
  synthetic oligonucleotide
gattttgagacacaacgtggctttcattgagtcactctctatg

SEQ ID NO: 173 - DNA, 20 bp, Artificial Sequence
  synthetic oligonucleotide
ctgcactggcgcacgtcgcc

SEQ ID NO: 174 - DNA, 20 bp, Artificial Sequence
  synthetic oligonucleotide
cgatggcagcgtggaagtgg

SEQ ID NO: 175 - DNA, 20 bp, Artificial Sequence
  synthetic oligonucleotide
gcgaaaacttctccattgcc

SEQ ID NO: 176 - DNA, 20 bp, Artificial Sequence
  synthetic oligonucleotide
cagcgggccataatcccttg

SEQ ID NO: 177 - DNA, 43 bp, Artificial Sequence
  synthetic oligonucleotide
cttcacgaggcagacctcagcgcctaacatgacatgaccatcc

SEQ ID NO: 178 - DNA, 44 bp, Artificial Sequence
  synthetic oligonucleotide
gattttgagacacaacgtggctttcatcatggccccctaattcg

SEQ ID NO: 179 - DNA, 20 bp, Artificial Sequence
  synthetic oligonucleotide
ccagtatattcaacaggggg

SEQ ID NO: 180 - DNA, 20 bp, Artificial Sequence
  synthetic oligonucleotide
cttcgccagttggatccagg

SEQ ID NO: 181 - DNA, 20 bp, Artificial Sequence
  synthetic oligonucleotide
caggctgcacaccagatggc

SEQ ID NO: 182 - DNA, 20 bp, Artificial Sequence
  synthetic oligonucleotide
cggaatttaccaacaaagag

SEQ ID NO: 183 - DNA, 42 bp, Artificial Sequence
  synthetic oligonucleotide
cttcacgaggcagacctcagcgcctaacaaggctgtactctg

SEQ ID NO: 184 - DNA, 42 bp, Artificial Sequence
  synthetic oligonucleotide
gattttgagacacaacgtggctttcacgggaacacctccttc

SEQ ID NO: 185 - DNA, 20 bp, Artificial Sequence
  synthetic oligonucleotide
cagacatttacgctattggc

SEQ ID NO: 186 - DNA, 20 bp, Artificial Sequence
  synthetic oligonucleotide
ggacctcgtcgaagcgaccg

SEQ ID NO: 187 - DNA, 20 bp, Artificial Sequence
  synthetic oligonucleotide
gatatatacggcagcaaaac

SEQ ID NO: 188 - DNA, 20 bp, Artificial Sequence
  synthetic oligonucleotide
ggcaatgcctatggctttac

SEQ ID NO: 189 - DNA, 43 bp, Artificial Sequence
  synthetic oligonucleotide
cttcacgaggcagacctcagcgcctaaggggtgaatcttgatg

SEQ ID NO: 190 - DNA, 44 bp, Artificial Sequence
  synthetic oligonucleotide
gattttgagacacaacgtggctttcatcgttacagacctttatg

SEQ ID NO: 191 - DNA, 20 bp, Artificial Sequence
  synthetic oligonucleotide
gcgattatatttagtgtgcg

SEQ ID NO: 192 - DNA, 20 bp, Artificial Sequence
  synthetic oligonucleotide
ctgaccagataatttcgccc

SEQ ID NO: 193 - DNA, 20 bp, Artificial Sequence
  synthetic oligonucleotide
cagctgtgcttcatgcttag

SEQ ID NO: 194 - DNA, 20 bp, Artificial Sequence
  synthetic oligonucleotide
gccggctggactggataacc

SEQ ID NO: 195 - DNA, 42 bp, Artificial Sequence
  synthetic oligonucleotide
cttcacgaggcagacctcagcgcctaagcattaaccctcatt

SEQ ID NO: 196 - DNA, 43 bp, Artificial Sequence
  synthetic oligonucleotide
gattttgagacacaacgtggctttcataatagtggccttatgc

SEQ ID NO: 197 - DNA, 20 bp, Artificial Sequence
  synthetic oligonucleotide
catgaagcagcctgccgggg

SEQ ID NO: 198 - DNA, 20 bp, Artificial Sequence
  synthetic oligonucleotide
gacaaacgtgcaaacccaac

SEQ ID NO: 199 - DNA, 20 bp, Artificial Sequence
  synthetic oligonucleotide
gtcgaatgtaccggcacccc

SEQ ID NO: 200 - DNA, 20 bp, Artificial Sequence
  synthetic oligonucleotide
catcaacagtattgctttcc

SEQ ID NO: 201 - DNA, 45 bp, Artificial Sequence
  synthetic oligonucleotide
cttcacgaggcagacctcagcgcctgaaaggtgaagggatctgtc

SEQ ID NO: 202 - DNA, 43 bp, Artificial Sequence
  synthetic oligonucleotide
gattttgagacacaacgtggctttcataagcgatgttaaaaac

SEQ ID NO: 203 - DNA, 20 bp, Artificial Sequence
  synthetic oligonucleotide
gcgtgtcgtattcttcttgc

SEQ ID NO: 204 - DNA, 20 bp, Artificial Sequence
  synthetic oligonucleotide
cgcttcatctcactgaggac

SEQ ID NO: 205 - DNA, 20 bp, Artificial Sequence
  synthetic oligonucleotide
caaaaaattgtcggtcaggc

SEQ ID NO: 206 - DNA, 20 bp, Artificial Sequence
  synthetic oligonucleotide
gcatattcacagcctggttc

SEQ ID NO: 207 - DNA, 44 bp, Artificial Sequence
  synthetic oligonucleotide
cttcacgaggcagacctcagcgcctaattccgctctctggatag

SEQ ID NO: 208 - DNA, 44 bp, Artificial Sequence
  synthetic oligonucleotide
gattttgagacacaacgtggctttcatatttgctcctcaataac

SEQ ID NO: 209 - DNA, 20 bp, Artificial Sequence
  synthetic oligonucleotide
gtgaagatctggatgctgcc

SEQ ID NO: 210 - DNA, 20 bp, Artificial Sequence
  synthetic oligonucleotide
ggtgttatcgggcgtaatcg

SEQ ID NO: 211 - DNA, 20 bp, Artificial Sequence
  synthetic oligonucleotide
cgcaaacacggaacggtaac

SEQ ID NO: 212 - DNA, 20 bp, Artificial Sequence
  synthetic oligonucleotide
gagatcaccagtaccgaagc

SEQ ID NO: 213 - DNA, 47 bp, Artificial Sequence
  synthetic oligonucleotide
cttcacgaggcagacctcagcgcctaagaaggtcaaagctatatgaa

SEQ ID NO: 214 - DNA, 46 bp, Artificial Sequence
  synthetic oligonucleotide
gattttgagacacaacgtggctttcattattccgcctccagaacca

SEQ ID NO: 215 - DNA, 20 bp, Artificial Sequence
  synthetic oligonucleotide
ggtgatgactgcctttatcc

SEQ ID NO: 216 - DNA, 20 bp, Artificial Sequence
  synthetic oligonucleotide
catcttcagattacacgggc

SEQ ID NO: 217 - DNA, 20 bp, Artificial Sequence
  synthetic oligonucleotide
gaaccgtagccgtcgtctgc

SEQ ID NO: 218 - DNA, 20 bp, Artificial Sequence
  synthetic oligonucleotide
catcgtgtcggtaccgtggg

SEQ ID NO: 219 - DNA, 44 bp, Artificial Sequence
  synthetic oligonucleotide
cttcacgaggcagacctcagcgcctaatcctctattttaagacg

SEQ ID NO: 220 - DNA, 41 bp, Artificial Sequence
  synthetic oligonucleotide
gattttgagacacaacgtggctttcatagtcttccctcaag

SEQ ID NO: 221 - DNA, 20 bp, Artificial Sequence
  synthetic oligonucleotide
gtgatgtcttctattgacgg

SEQ ID NO: 222 - DNA, 20 bp, Artificial Sequence
  synthetic oligonucleotide
gttggcggaggctttatcag

SEQ ID NO: 223 - DNA, 20 bp, Artificial Sequence
  synthetic oligonucleotide
cgaccaaggtgccttgagtc

SEQ ID NO: 224 - DNA, 20 bp, Artificial Sequence
  synthetic oligonucleotide
gcagccgcgaacgctgtacg

SEQ ID NO: 225 - DNA, 46 bp, Artificial Sequence
  synthetic oligonucleotide
cttcacgaggcagacctcagcgcctaataccagctaactcaggttc

SEQ ID NO: 226 - DNA, 47 bp, Artificial Sequence
  synthetic oligonucleotide
gattttgagacacaacgtggctttattaaggaaggtgcgaacaagtc

SEQ ID NO: 227 - DNA, 20 bp, Artificial Sequence
  synthetic oligonucleotide
ctgctctttgttcttggtcg

SEQ ID NO: 228 - DNA, 20 bp, Artificial Sequence
  synthetic oligonucleotide
gcgcagggtcgcgattctcg

SEQ ID NO: 229 - DNA, 20 bp, Artificial Sequence
  synthetic oligonucleotide
gcgatggaagcgggctactc

SEQ ID NO: 230 - DNA, 20 bp, Artificial Sequence
  synthetic oligonucleotide
gttcacgcagctcaacgaag

SEQ ID NO: 231 - DNA, 44 bp, Artificial Sequence
  synthetic oligonucleotide
cttcacgaggcagacctcagcgcctgataccgatggaaagagtc

SEQ ID NO: 232 - DNA, 44 bp, Artificial Sequence
  synthetic oligonucleotide
gattttgagacacaacgtggctttcatttttgtcttccgggacc

SEQ ID NO: 233 - DNA, 20 bp, Artificial Sequence
  synthetic oligonucleotide
gaatggttaactcgcaggtg

SEQ ID NO: 234 - DNA, 20 bp, Artificial Sequence
  synthetic oligonucleotide
cctgatcctgtaaatccgtg

SEQ ID NO: 235 - DNA, 20 bp, Artificial Sequence
  synthetic oligonucleotide
cgctggtgaaatggatatgg

SEQ ID NO: 236 - DNA, 20 bp, Artificial Sequence
  synthetic oligonucleotide
gataaaaaagcgcctcttag

SEQ ID NO: 237 - DNA, 45 bp, Artificial Sequence
  synthetic oligonucleotide
cttcacgaggcagacctcagcgcctaagatagtgccctttttctg

SEQ ID NO: 238 - DNA, 44 bp, Artificial Sequence
  synthetic oligonucleotide
gattttgagacacaacgtggctttcattcctttgtcctctttgg

SEQ ID NO: 239 - DNA, 20 bp, Artificial Sequence
  synthetic oligonucleotide
gtttcgcgtactcgaaatgg

SEQ ID NO: 240 - DNA, 20 bp, Artificial Sequence
  synthetic oligonucleotide
cgataagatgttgacagagg

SEQ ID NO: 241 - DNA, 20 bp, Artificial Sequence
  synthetic oligonucleotide
ggaaaaagcagggcttaacg

SEQ ID NO: 242 - DNA, 20 bp, Artificial Sequence
  synthetic oligonucleotide
gtcaaatgcgtttgtttcgc

SEQ ID NO: 243 - DNA, 50 bp, Artificial Sequence
  synthetic oligonucleotide
cttcacgaggcagacctcagcgcctaagtaaaagcccggtcacattggac

SEQ ID NO: 244 - DNA, 44 bp, Artificial Sequence
  synthetic oligonucleotide
gattttgagacacaacgtggctttcatctgtgtctctgtatctg

SEQ ID NO: 245 - DNA, 20 bp, Artificial Sequence
  synthetic oligonucleotide
caagccctggaaggtcctgg

SEQ ID NO: 246 - DNA, 20 bp, Artificial Sequence
  synthetic oligonucleotide
catatctgccagttagttgc

SEQ ID NO: 247 - DNA, 20 bp, Artificial Sequence
  synthetic oligonucleotide
cgattaacggtggtatcaag

SEQ ID NO: 248 - DNA, 20 bp, Artificial Sequence
  synthetic oligonucleotide
ccgtgggcagagacacctgg

SEQ ID NO: 249 - DNA, 50 bp, Artificial Sequence
  synthetic oligonucleotide
cttcacgaggcagacctcagcgcctaagggattgtgcggatgatcacaac

SEQ ID NO: 250 - DNA, 45 bp, Artificial Sequence
  synthetic oligonucleotide
gattttgagacacaacgtggctttcatgatgctctcccaaatatg

SEQ ID NO: 251 - DNA, 20 bp, Artificial Sequence
  synthetic oligonucleotide
gcaaaggcgagtgtgagatg

SEQ ID NO: 252 - DNA, 20 bp, Artificial Sequence
  synthetic oligonucleotide
gagcggttaaaagagatcac

SEQ ID NO: 253 - DNA, 20 bp, Artificial Sequence
  synthetic oligonucleotide
ggctgcataaaacccatgcc

SEQ ID NO: 254 - DNA, 20 bp, Artificial Sequence
  synthetic oligonucleotide
cgactgatgttcatattcgc

SEQ ID NO: 255 - DNA, 44 bp, Artificial Sequence
  synthetic oligonucleotide
cttcacgaggcagacctcagcgcctgatgtgcattacttaaccg

SEQ ID NO: 256 - DNA, 44 bp, Artificial Sequence
  synthetic oligonucleotide
gattttgagacacaacgtggctttcatgaagatatcctttatgg

SEQ ID NO: 257 - DNA, 20 bp, Artificial Sequence
  synthetic oligonucleotide
gctaacctggatgtgctggg

SEQ ID NO: 258 - DNA, 20 bp, Artificial Sequence
  synthetic oligonucleotide
ggtaccggacatccggcaac

SEQ ID NO: 259 - DNA, 20 bp, Artificial Sequence
  synthetic oligonucleotide
ccggcagatcgccccccgcc

SEQ ID NO: 260 - DNA, 20 bp, Artificial Sequence
  synthetic oligonucleotide
ggtgttggcgctgatacatc

SEQ ID NO: 261 - DNA, 48 bp, Artificial Sequence
  synthetic oligonucleotide
cttcacgaggcagacctcagcgcctaagctttatcgaagcaaaataag

SEQ ID NO: 262 - DNA, 46 bp, Artificial Sequence
  synthetic oligonucleotide
gattttgagacacaacgtggctttcatcattttgtcctcattatac

SEQ ID NO: 263 - DNA, 20 bp, Artificial Sequence
  synthetic oligonucleotide
ccacgcctgtgaatcttccg

SEQ ID NO: 264 - DNA, 20 bp, Artificial Sequence
  synthetic oligonucleotide
ccagggttccagccttcctg

SEQ ID NO: 265 - DNA, 20 bp, Artificial Sequence
  synthetic oligonucleotide
ctgtaagcgccttgtaagac

SEQ ID NO: 266 - DNA, 20 bp, Artificial Sequence
  synthetic oligonucleotide
cgaagctgatgtttgcgtcc

SEQ ID NO: 267 - DNA, 45 bp, Artificial Sequence
  synthetic oligonucleotide
cttcacgaggcagacctcagcgcctaatgctggaaatactctatc

SEQ ID NO: 268 - DNA, 44 bp, Artificial Sequence
  synthetic oligonucleotide
gattttgagacacaacgtggctttcataaagcaacctcaataag

SEQ ID NO: 269 - DNA, 20 bp, Artificial Sequence
  synthetic oligonucleotide
cttaagcctcttctgtaatc

SEQ ID NO: 270 - DNA, 20 bp, Artificial Sequence
  synthetic oligonucleotide
ggcccgcgtgaatagtcagc

SEQ ID NO: 271 - DNA, 20 bp, Artificial Sequence
  synthetic oligonucleotide
ggggatgccattatggagtg

SEQ ID NO: 272 - DNA, 20 bp, Artificial Sequence
  synthetic oligonucleotide
caccaaacgactcagcatgg

SEQ ID NO: 273 - DNA, 45 bp, Artificial Sequence
  synthetic oligonucleotide
cttcacgaggcagacctcagcgcctagcggcccgggtattccggg

SEQ ID NO: 274 - DNA, 43 bp, Artificial Sequence
  synthetic oligonucleotide
gattttgagacacaacgtggctttcacgagtctttatgacctc

SEQ ID NO: 275 - DNA, 20 bp, Artificial Sequence
  synthetic oligonucleotide
ctgcattttctatttcgacg

SEQ ID NO: 276 - DNA, 20 bp, Artificial Sequence
  synthetic oligonucleotide
gaaccttgcgacgacttgcc

SEQ ID NO: 277 - DNA, 21 bp, Artificial Sequence
  synthetic oligonucleotide
cgatggcgataatatttcacc

SEQ ID NO: 278 - DNA, 21 bp, Artificial Sequence
  synthetic oligonucleotide
ccctgatactcaccaggcatc

SEQ ID NO: 279 - DNA, 40 bp, Artificial Sequence
  synthetic oligonucleotide
tgagtaggacaaatccgccgctaaaaaagcaggcttcaac

SEQ ID NO: 280 - DNA, 40 bp, Artificial Sequence
  synthetic oligonucleotide
gcgttttttattggtgagaattactaacttgagcgaaacg

SEQ ID NO: 281 - DNA, 20 bp, Artificial Sequence
  synthetic oligonucleotide
ggcgataaattgccgcatcg

SEQ ID NO: 282 - DNA, 22 bp, Artificial Sequence
  synthetic oligonucleotide
tgccaccatcaagggaaagccc

SEQ ID NO: 283 - DNA, 24 bp, Artificial Sequence
  synthetic oligonucleotide
cgctggcagtcagttcattaagcc

SEQ ID NO: 284 - DNA, 23 bp, Artificial Sequence
  synthetic oligonucleotide
gtctccagttcgctaacattaac

SEQ ID NO: 285 - DNA, 24 bp, Artificial Sequence
  synthetic oligonucleotide
ctaccgatcacagcggcgcgtccc

SEQ ID NO: 286 - DNA, 23 bp, Artificial Sequence
  synthetic oligonucleotide
gttccagccttcatctgatttgg

SEQ ID NO: 287 - DNA, 25 bp, Artificial Sequence
  synthetic oligonucleotide
ctggcaatcgttgttctgtcggctc

SEQ ID NO: 288 - DNA, 24 bp, Artificial Sequence
  synthetic oligonucleotide
gctccttcctgtgccagcgatgcg

SEQ ID NO: 289 - DNA, 26 bp, Artificial Sequence
  synthetic oligonucleotide
gaacagctctatgaagacttcttaac

SEQ ID NO: 290 - DNA, 27 bp, Artificial Sequence
  synthetic oligonucleotide
gctgcaggactttaacctgcttcacat

SEQ ID NO: 291 - DNA, 25 bp, Artificial Sequence
  synthetic oligonucleotide
gttggcgggtatcgggattggtgtc

SEQ ID NO: 292 - DNA, 24 bp, Artificial Sequence
  synthetic oligonucleotide
gtaatgcgattttcatcctgcacc

SEQ ID NO: 293 - DNA, 26 bp, Artificial Sequence
  synthetic oligonucleotide
ccctcatcgctgcggcgattttaagc

SEQ ID NO: 294 - DNA, 25 bp, Artificial Sequence
  synthetic oligonucleotide
ccggttacagaagtaatacggaaag

SEQ ID NO: 295 - DNA, 24 bp, Artificial Sequence
  synthetic oligonucleotide
ggctgctggtcgcaaataaatcag

SEQ ID NO: 296 - DNA, 24 bp, Artificial Sequence
  synthetic oligonucleotide
gcaaggatcaaacgtgctgtacgc

SEQ ID NO: 297 - DNA, 20 bp, Artificial Sequence
  synthetic oligonucleotide
atgaccagccacactggaac

SEQ ID NO: 298 - DNA, 20 bp, Artificial Sequence
  synthetic oligonucleotide
cttcctccccgctgaaagta

SEQ ID NO: 299 - protein, 219 aa, Escherichia coli
MDRIVSSSHDRTSLLSTHKVLRNTYFLLSLTLAFSAITATASTVLMLPSPGLILTLVGMYGLMFLTYKTANKPTGIISAFAFTGFLGYILGPILNTYLSAGMGDVIAMALGGTALVFFCCSAYVLTTRKDMSFLGGMLMAGIVVVLIGMVANIFLQLPALHLAISAVFILISSGAILFETSNIIHGGETNYIRATVSLYVSLYNIFVSLLSILGFASRD

SEQ ID NO: 300 - DNA, 660 bp, Escherichia coli
atggatcgtattgttagttcttcacatgaccgtacatcactgcttagcacccataaggtgctgcgtaatacctattttctgctgagcctgacgctggccttttcggcgattaccgcaactgccagtacggtgctgatgctgccatctccgggtctgattctgacgctggtgggtatgtatggtttgatgttcctgacctataaaacggcgaataagccgaccgggattatctccgcattcgcctttaccggttttctgggttatatcctcggacctattctgaacacctatctgtctgccggaatgggtgacgtaatcgctatggcactgggcggaacggcgttagtgttcttctgctgctctgcatatgtgctgaccacccgcaaagatatgtcgttcctcggcggtatgctgatggcgggtattgtggtggtgctgattggtatggttgcgaatatcttcctgcagctgcctgctctgcatctggcgatcagcgcggtcttcattctgatctcctctggcgctatcttgtttgaaaccagcaacatcattcatggcggtgagacgaactatattcgtgccacggttagcctgtatgtttcgctgtacaacatcttcgtcagcctgctgagcattctgggcttcgctagccgcgattaa

SEQ ID NO: 301 - protein, 179 aa, Escherichia coli
MNKSMLAGIGIGVAAALGVAAVASLNVFERGPQYAQVVSATPIKETVKTPRQECRNVTVTHRRPVQDENRITGSVLGAVAGGVIGHQFGGGRGKDVATVVGALGGGYAGNQIQGSLQESDTYTTTQQRCKTVYDKSEKMLGYDVTYKIGDQQGKIRMDRDPGTQIPLDSNGQLILNNKV

SEQ ID NO: 302 - DNA, 540 bp, Escherichia coli
gtgaataaatcaatgttggcgggtatcgggattggtgtcgcagctgcgctgggcgtagcggcagtggccagtctgaacgtgtttgaacggggcccgcaatacgctcaggttgtttctgcaaccccaatcaaggaaacggttaaaacaccgcgtcaggagtgtcgcaacgtcacagtgacccatcgtcgaccggtgcaggatgaaaatcgcattaccgggtcggtgctcggcgctgttgctggcggcgtgatagggcatcagtttggtggtggtcgcggtaaagatgtcgccactgttgtgggggcgctgggtggtggatatgccggtaaccagatccagggctctctccaggaaagcgatacttacacgactacgcaacagcgttgtaaaacggtgtatgacaagtcagaaaaaatgctcggttatgatgtgacctataagattggcgatcagcagggcaaaatccgcatggaccgcgatccgggtacgcagatcccgctagatagcaacgggcaactgattttgaataacaaagtataa

SEQ ID NO: 303 - protein, 84 aa, Escherichia coli
MEKNNEVIQTHPLVGWDISTVDSYDALMLRLHYQTPNKSEQEGTEVGQTLWLTTDVARQFISILEAGIAKIESGDFQVNEYRRH

SEQ ID NO: 304 - DNA, 255 bp, Escherichia coli
atggaaaaaaataatgaagtcattcagactcatccgctcgtagggtgggacatcagcaccgttgatagctatgatgcgctgatgttgcgtttgcactaccagaccccaaataagtccgagcaggaagggactgaagttggtcagacgctctggttaaccactgatgttgccagacaatttatttcgatattagaagcaggaatcgccaaaattgaatccggtgatttccaggtaaacgagtatcggcgtcattaa

(19) United States
(12) Patent Application Publication
(10) Pub. No.: US 2006/0134640 A1
(43) Pub. Date: Jun. 22, 2006

(54) Inhibiting the growth of bacterial biofilms

(75) Inventors: Jean-Marc Ghigo, Fontenay-aux-roses (FR); Christophe Beloin, Rambouillet (FR); Patricia Latour-Lambert, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris (FR)

(21) Appl. No.: 10/829,452

(22) Filed: Apr. 22, 2004

Related U.S. Application Data
(60) Provisional application No. 60/464,333, filed on Apr. 22, 2003.
(60) Provisional application No. 60/517,391, filed on Nov. 6, 2003.

(51) Int. Cl. C12Q 1/68

(52) U.S. Cl. 435/6

(57) ABSTRACT

The present invention provides targets and methods for inhibiting the development, formation and/or maturation of bacterial biofilms, and for detecting bacterial biofilms.

### CROSS-REFERENCE TO RELATED APPLICATIONS

The present invention claims the benefit of U.S. provisional application No. 60/464,333 filed Apr. 22, 2003 and U.S. provisional application No. 60/517,391 filed Nov. 6, 2003, the entire contents of both applications are incorporated herein by reference.

### BACKGROUND OF THE INVENTION

1. Field of the Invention

The present invention relates to the inhibition or reduction in bacterial biofilm growth and development.

2. Discussion of the Background

Surface attached, matrix-enclosed communities, called biofilms, cause serious economic and health problems due to biofilm-associated phenotypes such as antibiotic resistance or biofouling Costerton, J. W., Stewart, P. S. & Greenberg, E. P. (1999) Science 284, 1318-22. The inherent resistance to antimicrobial agents are the root of many persistent and chronic bacterial infections as nosocomial infections and legionaire's disease. The drastic phenotypic changes seen in biofilms led to the assumption that the physiological modifications necessary for planktonic bacteria to adopt the biofilm lifestyle must involve specific responses. However, biofilm physiology is still poorly understood and, whereas the early events of biofilm formation are well documented, little is known about the nature of the physiological changes and critical regulatory processes occurring inside mature biofilms. Global expression profiling comparing protein synthesis in Pseudomonas planktonic and biofilm bacteria suggested that a large number of genes could be differentially regulated during biofilm development (Sauer, K., Camper, A. K., Ehrlich, G. D., Costerton, J. W. & Davies, D. G. (2002) J Bacteriol 184, 1140-54; Sauer, K. & Camper, A. K. (2001) J Bacteriol 183, 6579-89; Whiteley, M., Bangera, M. G., Bumgarner, R. E., Parsek, M. R., Teitzel, G. M., Lory, S. & Greenberg, E. P. (2001) Nature 413, 860-4). Although these pioneering studies opened the way to the genetic characterization of the biofilm phenotype, extracting functional information from genomic approaches remains a challenge.

Escherichia coli K12, a widely used bacterial model, does not spontaneously form extensive biofilms. However, it has been previously shown that expression of pili from conjugative plasmids, which are widespread in natural bacterial populations, promotes the development of mature biofilms (Ghigo, J. M. (2001) Nature 412, 442-5). This raised the possibility of studying the genetic basis of the biofilm phenotype in E. coli K12 where expression profiling can be combined with the phenotypic analysis of a large set of deletion mutants.

In view of the above, there remains an urgent need to develop new strategies for combating the development of mature biofilms Based on the discovery of the genes involved in the development of mature biofilms, the present invention provides targets to disrupt the development, formation and/or maturation of bacterial biofilms, and molecular tools to characterize and detect mature biofilms.

### SUMMARY OF THE INVENTION

Thus, the present invention is based on the discovery of the unique expression of genes during the formation of bacterial biofilms thereby providing a target to reduce, ameliorate, attenuate, inhibit and/or treat biofilms.

Accordingly, one aspect of the present invention is to a method of treating, reducing, ameliorating, attenuating and/or inhibiting the formation of biofilms by targeting the specific genes that are involved in the formation of the biofilm. These methods can be accomplished by contacting an already formed biofilm and/or a sample, surface or other substrate that may be susceptible to biofilm formation with one or more inhibitors of those genes.

In another aspect of the present invention, methods of screening for substances that inhibit the genes involved in biofilm formation is also provided.

In another aspect of the present invention, a polynucleotide library which is useful for molecular characterization of a mature bacterial biofilms is also provided.

In another aspect of the present invention, using the libraries to detect mature bacterial biofilms is also provided.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1: Function of genes over-expressed in TG1 biofilm versus exponential growth phase

This figure summarizes the data presented in Table 3. The genes have been classified according to the COGs functional categories annotation system. Large and medium size numbers indicate the total number of E. coli biofilm-induced genes into each class or sub-class of indicated functions. Genes are indicated only when their expression level in biofilm differed by at least a two-fold factor (≧2). Numbers within brackets indicate the rank as over-expressed genes; 1=most expressed gene in TG1 E. coli biofilm.

FIG. 2: Correlation of macroarray and quantitative real-time PCR results

The calculated macroarray and Q-RT-PCR ratios of the expression of 7 genes in TG1 biofilm relative to exponential growth phase were log transformed, and values were plotted against each other to evaluate their correlation. The correlation coefficient was deduced from a linear regression of the plotted values.

FIG. 3: Biofilm phenotype of selected deletion mutants

Mature biofilm development of E. coli TG1 (wt) compared with a selection of deletion mutants of genes over-expressed in TG1 biofilm.

A: For each mutant phenotype analysis, the extent of biofilm formation is shown in the bottom part of the micro-fermenter and on the removable glass slide. A typical experiment is shown.
B: Graphical comparison of biofilm formation relative to wild type from the mutants presented in A. Data represents the average of three independent experiments for each mutant. The level of biofilm formed by wt TG1 biofilm was set to 100%.

FIG. 4: Functional profiling of E. coli biofilm: flow chamber analysis
A. Spatial distribution of biofilm formation for E. coli TG1 and selected TG1 deletion mutants expressing Gfp. Biofilms were grown in flow chambers. Biofilm development was monitored by SCLM at the indicated times after inoculation (20 h, 45 h, 70 h, 95 h). Micrographs represent simulated three-dimensional images. Images inseted into 70 h and 95 h of ycfJ correspond to rare area where the biofilm was more developed.
B. COMSTAT analysis of biofilm structures. Diagrams and standard deviations (numbers indicated in the individual columns) of biomass and substrate coverage from biofilms of E. coli TG1 and TG1 deletion mutants were determined by the COMSTAT program at four different time points (20 h, 45 h, 70 h, 95 h). Values are means of data from 12 image stacks (6 image stacks from two independent channels). The biomass is in the unit μm³/μm². The substratum coverage values are relative (1 represents total coverage).

FIG. 5: A comparison of biofilm formation capacity of mutants in the E. coli cpx and rpoE envelope stress pathways

Biofilm development comparison of TG1 and TG1 deletion mutants in micro-fermenters. The average of at least four experiments was plotted in the histogram. The level of biofilm formed by wt TG1 biofilm was set to 100%.

FIG. 6: Comparison of TG1 and TG1 ΔcpxP biofilm structure

Phenotypic analysis of the structure of TG1 and TG1ΔcpxP biofilms grown in micro-fermenter.

A: General view of the bottom part of the fermenter.
B: Macroscopic biofilm grown on the internal glass slide, removed from the fermenter shown in panel A.
C: Close-up on the biofilm shown in panel B.
D: transverse section of TG1 and TG1ΔcpxP biofilm.
E and F: detailed X50 and X 10000 electron micrographs of TG1 and TG1 cpxP biofilm structure.

FIG. 7: COG functional classes for genes under-expressed in TG1 biofilm versus exponential growth phase.

This figure summarizes the data presented in Table 4. The genes have been classified according to the COGs functional categories annotation system. Large and medium size numbers indicate the total number of E. coli genes falling into each class or sub-class of function. Genes are indicated only when their expression level in biofilm differed by at least a two-fold factor (≦0.5). Numbers within brackets indicate the rank as under-expressed genes; 1=most repressed gene in TG1 biofilm.

FIG. 8: Functional profiling of mature E. coli biofilm: biofilm formation in microfermenters.

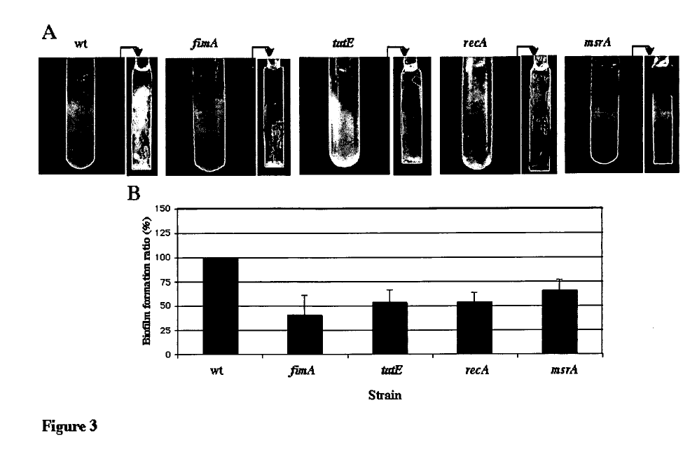

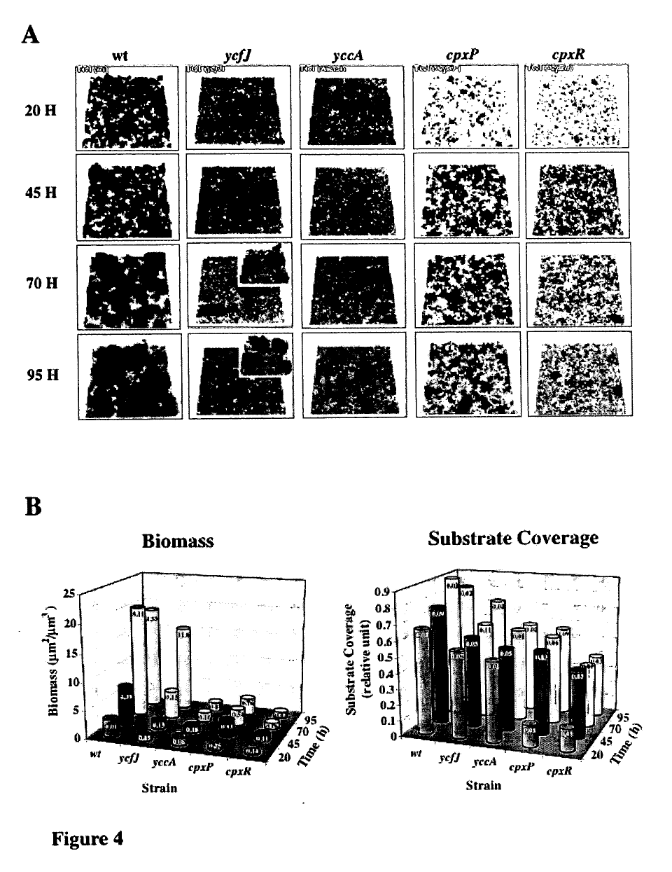

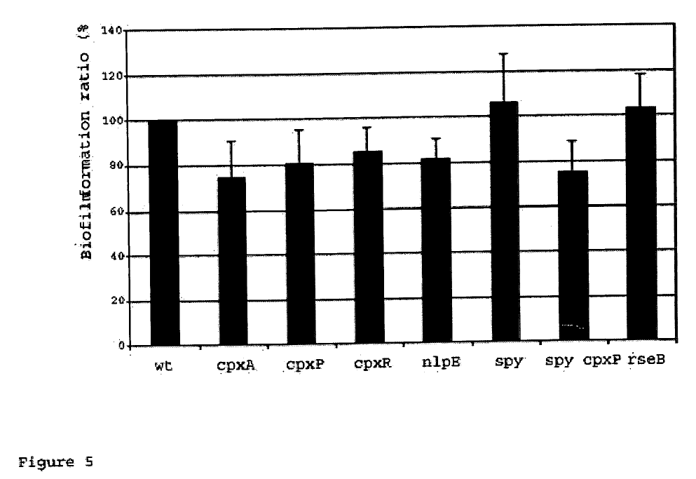

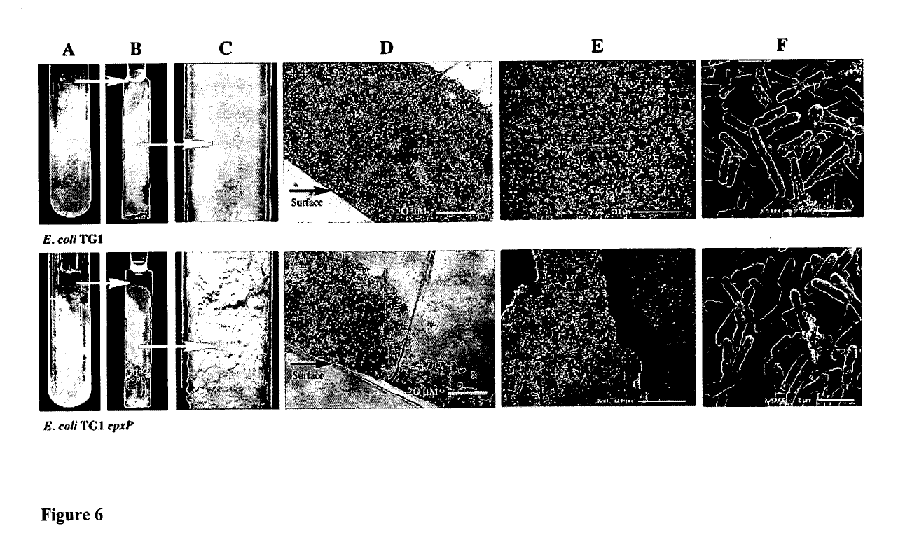

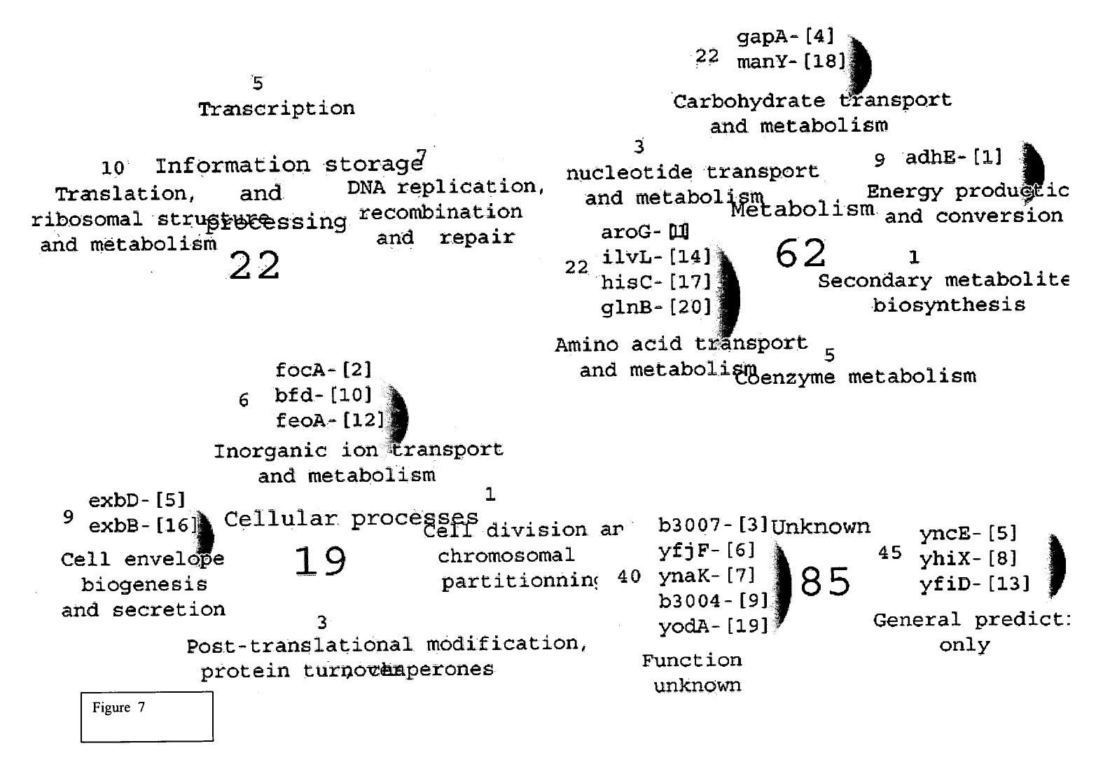

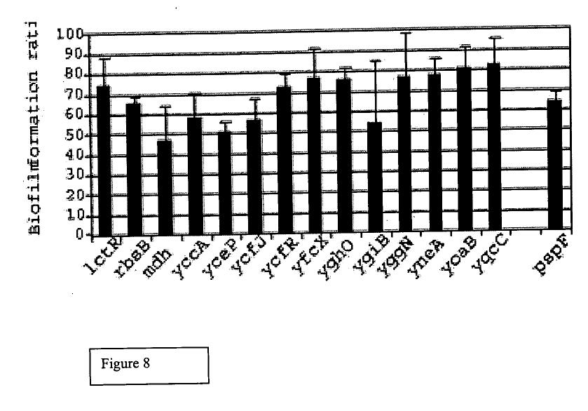

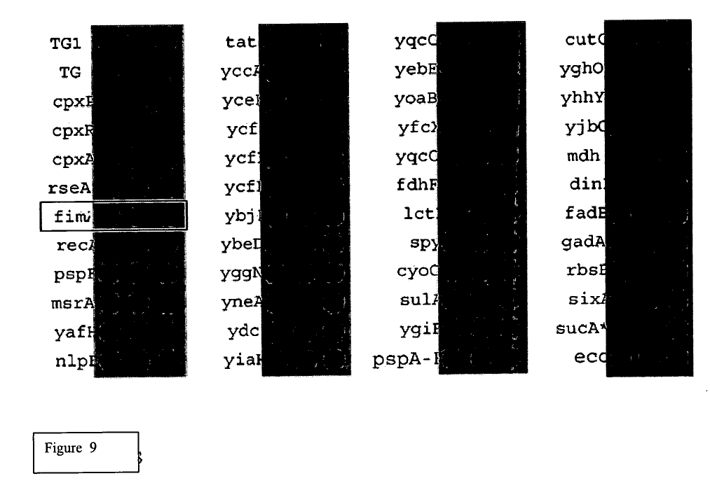

Comparison of mature biofilm development in micro-fermenters of wild type E. coli TG1 with TG1 mutants in the genes found to be induced by over a two-fold factor in TG1 biofilm. This figure complements the FIG. 3. The far right of the panel describes the analysis of biofilm development of a pspF mutant, a constitutively expressed positive regulator of the pspABCDE operon. The data represent the average of at least three independent experiments for each mutant. Wild type TG1 biofilm formation was set to 100.

FIG. 9: Functional profiling of early steps in E. coli biofilm formation

Comparison of the early adhesion ability of TG1 mutants in genes identified as over-expressed in mature TG1 versus exponential growth phase or analyzed in this study as visualized by crystal violet staining in a static microtiter plate-based assay. E. coli TG (M63B1 glucose medium supplemented with proline) adheres poorly in this assay. TG1 fimA (boxed) displays an expected reduced early adhesion capacity. Stars (*) correspond to TG1 mutants with a growth impairment leading to a non meaningful reduction of adhesion in this early biofilm assay.

### DETAILED DESCRIPTION OF THE INVENTION

The formation of biofilms results in a major lifestyle switch that is thought to affect the expression of multiple genes and operons. Using DNA arrays to study the global effect of biofilm formation on gene expression, the inventors have demonstrated that in biofilms, 1.9% of the genes showed a consistent up or down-regulation by a factor greater than two, and that 10% of the E. Coli genome is significantly differentially expressed including genes of unknown function, stress-response genes as well as energy production and envelope biogenesis functions. The inventors provide evidence that the expression of stress envelope response genes, such as the psp operonor elements of the cpx pathway, is a general feature of E. coli biofilms. Using gene disruption of 53 of the genes showed that 17 of the genes are required for the formation of mature biofilm. This includes 11 genes of previously unknown function.

Thus, the genes involved in biofilm formation and useful as targets for identifying substances that inhibit biofilm formation are those described herein, for example, including lctR, recA, mdh, rbsB, msrA, finA, tatE, pspF, cpxP, spy, ycfJ, ycfR, yoaB, yqcC, yggN, ymcA, yccA, yfcx, yghO, yceP, and ycuB. Preferably, the genes involved in biofilms formation are one or more of yccA, (SwissProt accession number—P06967; GenBank number—g1787205, the amino acid sequence is shown as SEQ ID NO:299 and the nucleotide sequence encoding the protein is shown in SEQ ID NO:300), ycfJ, (SwissProt accession number—P37796;GenBank number—g1787353, the amino acid sequence is shown as SEQ ID NO:301 and the nucleotide sequence encoding the protein is shown in SEQ ID NO:302), and yceP, (SwissProt accession number—P75927;GenBank number—g1787299, the amino acid sequence is shown as SEQ ID NO:303 and the nucleotide sequence encoding the protein is shown in SEQ ID NO:304).

As used herein, the term “polynucleotide” refers to a polymer of RNA or DNA that is single-stranded, optionally containing synthetic, non-natural or altered nucleotide bases. A polynucleotide in the form of a polymer of DNA may be comprises of one or more segments of cDNA, genomic DNA or synthetic DNA.

The term “subsequence” refers to a sequence of nucleic acids that comprise a part of a longer sequence of nucleic acids.

In a further embodiment of the invention, the proteins are at least 70%, preferably at least 80%, more preferably at least 90% identical to the sequences identified above. In another embodiment, the genes and thus gene products that are to be inhibited are encoded by polynucleotide sequence with at least 70%, preferably 80%, more preferably at least 90%, 95%, and 97% identity to the sequences described above, these polynucleotides will hybridize under stringent conditions to the coding or non-coding polynucleotide sequence above. Preferably, these homologous sequences would have the same or similar activity to the sequences specifically identified above.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions will be those where hybridization in 50% formamide, 1 M NaCl, 1% SDS at 37° C., and a wash in 0.1×SSC at 60 to 65° C. (see Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995)). Amino acid and polynucleotide identity, homology and/or similarity can be determined using the BLAST algorithm. Preferably, these homologous sequences would have the same or similar activity to the sequences specifically identified above.

In one embodiment, the present invention provides methods of reducing, inhibiting, ameliorating, and/or treating bacterial biofilms, such as E. coli biofilms, by inhibiting, reducing, and/or attenuating the genes and/or gene products, e.g, messenger RNA and proteins encoded thereby, described herein as being involved in biofilm formation.

By “treating” is meant the slowing, interrupting, arresting or stopping of the progression of the biofilm growth and does not necessarily require the complete elimination of the biofilm. “Preventing” or “ameliorating” is intended to include the prophylaxis of the biofilm development and/or growth, wherein “prophylaxis” is understood to be any degree of inhibition on the biofilm development and/or growth, including, but not limited to, the complete prevention of biofilm development and/or growth. The substances which inhibit the gene(s) described herein are collectively termed “biofilm inhibitor(s).” In one embodiment, the biofilm inhibitor(s) decrease the ability of the biofilm to develop and/or mature at least by 1%. In another embodiment, the decrease is at least by 5%, 10%, 15%, 20%, 30%, 35%, 40%, etc.

To effectuate the inhibition of biofilms, a surface, and/or sample (collectively termed “at least one substrate”) on which a biofilm has begun to develop can be contacted with one or more of the biofilm inhibitors thereby inhibiting the biofilm formation. In an alternative embodiment, the at least one substrate on which a biofilm has already formed or developed can be contacted with one or more of the biofilm inhibitors such that biofilm becomes less prevalent or completely disappears from the substrate. In an alternative embodiment, the at least one substrate in which a biofilm has not begun to develop but is susceptible to biofilm formation can be pretreated with one or more of the biofilm inhibitors to inhibit the formation of the biofilm on the at least one substrate. The substrate as used herein refers to any surface, liquid or solid, on which a biofilm develops, has developed, or is susceptible to biofilm formation.

The biofilm inhibitors can be any substance, chemical, and/or biological materials that inhibit the development and/or formation of the biofilm in an appreciable manner as described herein. For example, antibodies that specifically bind to and inhibit the activity of proteins that are encoded by the genes described herein can be used to inhibit the development and/or formation of the biofilm. Polyclonal, monoclonal and/or fragments (e.g., Fab fragments) of antibodies that specifically bind to the proteins of the genes described herein may be used so long as they inhibit the function of the gene products according to the disclosure herein. Obtaining polyclonal, monoclonal and/or functional fragments thereof is conventional and is described, for example, in Harlow and Lane “Using Antibodies: A Laboratory Manual”© Cold Spring Harbor Laboratory Press, Cold Spring Harbor, N.Y. (1999).

An effective amount of the inhibitors as described herein can be used either singularly or in combination and should be used in an amount that results in some inhibition of biofilm development and/or growth When the inhibitors are administered in combination, they may be premixed, administered simultaneously, or administered singly in series.

In another aspect of the present invention, methods to identify substances, agents, compounds and/or chemicals (collectively termed “inhibitors”) that reduce, inhibit, ameliorate and/or treat the development of and/or formation of biofilms. Such methods are preferably accomplished by targeting one or more of the genes involved in biofilm development as described herein. In one embodiment of this aspect, the gene (or genes) are expressed in host cell, preferably a bacterial cell such as E. coli, and the ability of the inhibitor to affect the gene and/or protein are assessed. For example, levels of transcription can be measured using conventional DNA and/or RNA probing techniques, such as PCR and other hybridization assays. Thus, the cell expressing the one or more protein encoded by the genes described herein is contacted with the inhibitor and the relative level of transcription is measured in relation to the cell before contacting with the inhibitor; and/or compared to a cell which similarly expresses the protein(s) and which was not contacted with the inhibitor. In a similar manner, levels of protein expressed in cell can be assessed, comparing contacted and uncontacted cells, using protein analytical techniques known in the art.

Screening for the inhibitors can also be accomplished by testing the effects of the inhibitor(s) on the development and/or growth of the biofilm as described herein. Once identified, the inhibitors can be used to reduce, inhibit, ameliorate and/or treat the development of and/or formation of biofilms as described herein.

The inhibitors may be formulated or combined with any acceptable carrier, such as buffered saline or other buffered solution.

In another aspect of the invention, a polynucleotide library is provided that is useful in the molecular characterization of a mature bacterial biofilm, which comprises a pool of polynucleotide sequences or subsequences thereof wherein said sequences or subsequences are overexpressed in mature bacterial biofilms. The polynucleotide sequences or subsequences may be immobilized on a solid support in order to form a polynucleotide array. As used herein, the term “immobilized on a support” means bound directly or indirectly thereto including attachment by covalent binding, hydrogen bonding, ionic interaction, hydrophobic interaction or otherwise. The solid support can be a nylon membrane, glass slide, glass beads, and/or a silicon chip. Thus, in another embodiment, a polynucleotide array is provided which is useful to detect a mature bacterial biofilm and which comprises an immobilized polynucleotide library as described above.

The immobilized polynucleotide library and array can be used for detecting differentially expressed polynucleotide sequences which are specifically correlated with a mature bacterial biofilms. In this method a polynucleotide sample is obtained, and labeled by reacting the polynucleotide sample with a labeled probe immobilized on a solid support wherein said probe comprises any of the polynucleotide sequences of the polynucleotide library as described above or an expression product encoded by any of the polynucleotide sequences; and detecting a polynucleotide sample reaction product. The method can be used for detecting mature bacterial biofilms, such as, an Escherichia coli biofilm.

In another embodiment of the method, a control polynucleotide sample, which is labeled, is employed for comparing the amount of polynucleotide sample reaction product to the amount of the control sample reaction product. In another embodiment of the method, RNA or mRNA is isolated from the polynucleotide sample, and which may be reverse transcribed to yield a cDNA molecule.

The labeling reaction can be performed by hybridizing the polynucleotide sample with the labeled probe. The label can be radioactive, colorimetric, enzymatic, molecular amplification, bioluminescent or fluorescent. Detection can then be performed as known in the art.

In another embodiment, where the product encoded by any of the polynucleotide sequences or subsequences is employed, the detection can be based on a receptor-ligand reaction.

In another aspect of the present invention, a method of detecting significantly overexpressed genes correlated with a mature bacterial biofilms can be performed. As used herein, “significantly overexpressed” means that the gene or expression product detected is expressed in by a factor of 2 or greater compared to a bacterial cell which is not in a biofilm or begun to develop biofilms characteristics. This method comprises detecting at least one polynucleotide sequence or subsequence of a polynucleotide library as described above or detecting at least one product encoded by said polynucleotide library in a sample obtained from a patient. In another embodiment of this method, an amount of the at least one polynucleotide sequence or subsequence or product encoded by said polynucleotide sequence is compared with an amount of the polynucleotide sequence or subsequence or product encoded by said polynucleotide sequence or subsequence obtained from a control sample. Extracted mRNA may also be used, which can be reverse transcribed into a cDNA molecule. In another embodiment of this method, the at least one polynucleotide sequence or subsequence can be hybridized with mRNA or cDNA from the polynucleotide sample using, for example, the labeling and detection described above. In another embodiment, of this method where the product encoded by any of the polynucleotide sequences or subsequences is employed, the detection can be based on a receptor-ligand reaction.

Preferably, the sequences or subsequences correspond substantially to the polynucleotide sequences of the following genes: rne, lctR, dinI, glpQ, mdh, sixA, lamB, rbsB, gadA, pspA, pspB, pspC, pspD, tatE, cpxP, rseA, rpoE, spy, yebE, yqcC, yfcX, yjbO, yceP, and ygiB. In another embodiment, the library further comprises polynucleotide sequences or subsequences thereof of the following genes: recA, msrA, fimA, pspF, ycfJ, ycfR, yoaB, yggN, yneA, yccA, yghO. In another embodiment, the library further comprises polynucleotide sequences or subsequences thereof of the following genes: RplY, recA, cyoD, sucA, fdhF, cyoC, nifU, sucD, sfsA, nifS, fadB, ucpA, ftsL, sulA, eco, msrA, pspD, fimA, fimI, pspE, pspF, cutC, sodC, rseB, ycfJ, ycfR, yoaB, yhhY, yggN, yneA, ybeD, ydcI, yddL, yccA, yrdD, ybjF, yihN, 1228, ycfL, yiaH, yqeC.

In another embodiment, the library further comprises polynucleotide sequences or subsequences thereof of the following genes: lysU, miaA, rluC, rplY, crl, cspD, dniR, fruR, idnR, lacI, nac, rnk, rpoS, ttk, b0299, dinG, dinP, exo, intA, recA, recN, sbmC, xthA, aceA, aceB, aldA, atpA, cyoA, cyoC, cyoD, dctA, fdhF, fdoG, glpD, glpK, nifU, pckA, sdhB, sdhD, sucA, sucB, sucD, xdhD, agp, gcd, glgS, glpX, malE, malF, malS, mglA, mglB, mrsA, pgm, rbsC, rbsD, sfsA, ansB, argC, argR, idnD, leuD, metH, nifS, putP, metK, pnuC, ubiE, fabA, fadB, fadE, fadL, pgpA, pssA, uppS, idnO, ucpA, ftsL, sulA, dnaJ, dnaK, eco, fkpA, glnE, htpG, htpX, msrA, amiB, ddg, fhiA, fimA, fimI, htrL, lepB, mraW, nlpB, nlpC, ompC, ompG, pspE, pspF, chaA, chaC, cutC, cysP, cysU, fur, modA, modB, modC, modE, sodC, trkH, rseB, ycfJ, ycfR, yoaB, yhhY, yggN, yneA, ybeD, ydcI, yddL, yccA, yrdD, ybjF, yihN, ycfT, yeeF, yfiE, yeeD, yliH, yfcM, ybiX, yfhF/nifA, ygfQ, ybhR, ybdH, yihR, ydcT, ygiS, ybaZ, ydaM, tfaR, yceL, yheT, yjdC, ybiW, ybiF, ynaI, yceE, yhdP, ygjE, csiE, yfdE, yeeE, yegQ, glcA, yfdW, yfeT, ygjK, ydeW, b1228, ycfL, yghO, yiaH, yqeC, ycfT, yhjJ, yceB, ybiX, ygiQ, yagV, yoeA, ybhQ, ybcI, ybbF, ybgI, yncH, yfbM, yjiM, yjfO, ychN, ynaC, ymfE, yfcN, yrbC, yfdQ, yfeY, ygiM, yhgA, yhjQ, yfcF, yfcI, yjiD, yfbP, yphB, yfbN, ylbH, ybhM, yrbL, yjfY, ynfA, yajI, yedI, yafZ, yjjU, yfhH, yafN, yrbE, yfgC, yfjQ, ycaK, yfeS, b4250, ybgA, yeeA, ypfI, b2394, yegK, ybcJ, yhiN, ypfG, ydiY, yjjJ, ycaP, yfgJ.

In a preferred embodiment of the library, the biofilms is a Escherichia coli biofilms.

### EXAMPLES

### Experimental Procedures

Bacterial Strains and Culture Conditions

Bacterial strains used in this work are described in Table 2. All experiments were performed in 0.4% glucose M63B1 minimal medium at 37° C. except flow chamber experiments that were performed at 30° C. in 0.02% glucose FAB minimal medium. Proline was added at 400 μg/ml for TG growth.

Early Adhesion and Biofilm Formation Assay

Microtiter plate assays were performed as described in (O'Toole, G. A., and Kolter, R. (1998) Mol Microbiol 30: 295-304). Biofilm development comparisons in aerated micro-fermenters were conducted as described in (Ghigo, J. M. (2001) Nature 412: 442-445.). The biofilms formed on the removable glass slide were photographed and then resuspended in 10 ml of M63B1 minimal medium. The optical density at 600 nm (OD₆₀₀) of the resuspension was then measured. After 24 hours the average resuspended E. coli TG1 biofilm biomass reached OD₆₀₀=5. Each mutant was tested in at least 3 independent experiments alongside with the control strain TG1.

Macroarray Analysis

Genomic expression profiles were performed on E. coli TG1 and TG strains grown in 0.4% glucose M63B1 at 37° C. either as planktonic cultures or mature biofilms. Planktonic cultures were realized in agitated Erlenmeyer flasks (main experiment) or aerated micro-fermenters, both in exponential phase OD₆₀₀˜0.6 or stationary phase OD₆₀₀˜3. Mature biofilms were grown in aerated micro-fermenters (8 and 5 day old biofilms for TG1 and TG respectively). For all conditions, the equivalent of 15 OD₆₀₀ of bacterial cells was collected. The cells were then broken in a Fast Prep apparatus (Bio 101). Total RNA was extracted by Trizol (Gibco-BRL) treatment. Genomic DNA was degraded using the DNA-free™ kit (Ambion). Radioactively labeled cDNAs, generated by using E. coli K12 CDS-specific primers (SIGMA-GenoSys), were hybridized to E. coli K12 panorama gene arrays containing duplicated spots for each of the 4,290 predicted E. coli K12 ORFs (SIGMA-GenoSys). The intensity of each dot was quantified with the XDOTSREADER software (Cose) as described in (Hommais, (2001) Mol Microbiol 40: 20-36). Experiments were carried out using three independent RNA preparations of TG1 planktonic flask cultures versus TG1 biofilm. For the F free TG experiment and the TG1 planktonic fermenter versus TG1 biofilm experiments, two independent RNA preparations were used. Each hybridization with each independent sample was carried out with 1 μg and 10 μg of total RNA. Comparison of the signal intensity of arrays from duplicates or from independent hybridizations showed that the results were highly reproducible (data not shown).

Statistical Analysis of the Macroarray Data

Genes that were statistically significantly over- and under-expressed were identified using the non-parametric Wilcoxon rank sum test. For each gene, the expression in E. coli TG1 flask exponential and stationary planktonic cultures (n=10 and n=12, respectively), TG flask planktonic cultures (n=4), TG1 fermenter planktonic culture (n=4) and TG1 biofilm (n=10) or TG biofilm (n=4) were compared. Analyses were performed with one tailed tests. Genes were considered to be statistically significantly over- or under-expressed when p<0.05. Low (less than 0.01) or negative levels of expression were removed from the analysis.

Disruption of Genes Identified Through Macroarray Analysis

fimA, msrA, recA, cpxA and pspF mutants were transferred to TG1 by P1 transduction. For the other genes, a non-polar mutation that deletes the entire target gene from the initiation to the stop codon, was created by allelic exchange with the non-polar aphA gene cassette from Tn903. We used a 3-step PCR procedure as described in (Chaveroche, M. K., Ghigo, J. M., and d'Enfert, C. (2000) Nucleic Acids Res 28: E97; Derbise, A., (2003) FEMS Immunol Med Microbiol 38: 113-116) and detailed at previously.

The primers used to inactivate the 54 genes presented in this study, as well as nlpE and cpxR genes, are described in Table 6.

Quantitative RT-PCR

Quantitative reverse transcription PCR (Q-RT-PCR) was used to confirm the DNA macroarray data. Total RNAs used for macroassay were used for real-time PCR and RT-PCR. PCR and RT-PCR were performed using a light-cycler (Roche Diagnostics). The RNA preparation was subjected twice to DNase I (Roche Diagnostics) treatment for 30 min at room temperature to remove any contaminating genomic DNA. The enzyme was then inactivated 15 min at 65° C. in the presence of 2.5 mM EDTA. Samples were checked for residual genomic DNA by real-time PCR using the cpxP-RT-5 and cpxP-RT-3 primers (see Table 7). Reactions were performed in a 20 μl reaction volume using LightCycler FastStart DNA master SYBR Green I (Roche Diagnostics) according to the manufacturer's instructions. RNA samples were considered to be free of genomic DNA if no amplification was detected after at least 35 cycles of amplification. Quantitative RT-PCR reactions were performed twice with two independent RNA preparations and using primers specifics for several biofilm up-regulated genes (see Table 7) or control 16S rDNA primers (TM1, 5′-ATGACCAGCCACACTGGAAC-3′ (SEQ ID NO:297) and TM2, 5′-CTTCCTCCCCGCTGAAAGTA-3′ (SEQ ID NO:298)) with 50 ng of total RNA. Control 16S rDNA primers were always used to ensure the same quantity of total RNA in each reaction sample. Quantification of mRNA or 16S rRNA (as control) was done using RNA master SYBR Green I (Roche Diagnostics) according to the manufacturer's instructions. Amplification of a single PCR product was confirmed by fusion curve analysis and electrophoresis on 2% agarose gels.

Construction of GFP-Tagged Strains

The strain TG1gfp was constructed by integration at the λ-att site of a bla-gfpmut3 cassette amplified from plasmid pZER1-GfpSal using a 3-step PCR procedure (Table 4S) as described in (Chaveroche, M. K., Ghigo, J. M., and d'Enfert, C. (2000) Nucleic Acids Res 28: E97; Derbise, A., (2003) FEMS Immunol Med Microbiol 38: 113-116). Plasmid pZER1-GfpSal is a gift from C. C. Guet where the gfpmut3 gene (Cormack et al., 1996 Gene 173: 33-38) is controlled by the lambda right promoter. Strains TG1gfpΔycfJ, TG1gfpΔyccA, TG1gfpΔcpxP and TG1gfpΔcpxR were constructed by P1vir transduction into TG1gfp.

Flow Chamber Experiments

Biofilms were cultivated at 30° C. in three-channel flow cells with individual channel dimensions of 1×4×40 mm. The flow system was assembled and prepared as previously described (Christensen et al., 1999, Methods Enzymol 310: 20-42). A microscope glass cover slip (Knittel 24×50 mm st1; Knittel Gläser) was used as substratum for biofilm growth.

Inocula were prepared as follows: 16-20 h old overnight cultures in LB supplemented with the appropriate antibiotics were harvested and resuspended in 0.9% NaCl. 250 μL of OD₆₀₀-normalized dilutions in 0.9% NaCl (OD₆₀₀=0.05) were injected into each flow channel after medium flow was arrested. Flow was started 1 h after inoculation at a constant rate of 3 mL h⁻¹ using a Watson Marlow 205S peristaltic pump.

Microscopy and Image Analysis

Biofilm development in micro-fermenters was recorded with a Nikon Coolpix 950 digital camera. Transmission and scanning laser electronic microscopy were performed on biofilm grown in micro-fermenters on thermanox slides (Nalgene) attached to the internal removable glass slide and treated as described in (Prigent-Combaret et al., 2000, J Bacteriol 181: 5993-6002.).

For flow chamber experiments, microscopic observations and image acquisitions were performed on a Zeiss LSM510 Scanning Confocal Laser Microscope (Carl Zeiss, Jena, Germany). Images were obtained using a 40×/1.3 Plan-Neofluar oil objective. Simulated three-dimensional images were generated by using the IMARIS software package (Bitplane AG, Zürich, Switzerland). Images were further processed for display using Adobe Photoshop. For COMSTAT analysis (Heydorn et al., 2000, Microbiology 146 (Pt 10): 2395-2407) and quantification of the E. coli biofilm development with the wild type and the different mutants, each strain was grown in two separate channels, and six image stacks were acquired randomly down through each channel at different time points (20 h, 45 h, 70 h and 95 h after inoculation).

### Results

Production of Mature E. coli Biofilms

The capacity of different E. coli K12 strains to form mature biofilms was tested in M63B1-glucose minimal medium in a micro-fermenter-based continuous flow culture system (Ghigo, 2001, Nature 412: 442-445.). Most of the strains tested formed only thin biofilms after 2 to 5 days. However, high biomass and thick biofilm production (>200 μM) was reproducibly achieved using E. coli TG1, a strain carrying the F conjugative plasmid previously shown to promote biofilm formation (Ghigo, 2001, Nature 412: 442-445.; Reisner et al., 2003, Mol Microbiol 48: 933-946). To identify E. coli genes that are differentially expressed in mature biofilms, we compared 8 day-old TG1 biofilms to late exponential TG1 planktonic (OD=0.6) or stationary phase cultures (OD=3). Whereas in agitated flask and planktonic culture conditions, no surface adhesion was observed, a significant amount of contaminating biofilm formation occurred in planktonic TG1 continuous cultures grown in fermenters. This led us, in the main experiment described in this study, to compare planktonic cultures grown in agitated flasks to TG1 biofilms grown in fermenters. However, differential gene expression between planktonic and biofilm bacteria both grown in fermenters was also investigated (see discussion).

Biofilm Formation Has a Global Impact on Gene Expression When Compared to Exponential Growth Phase

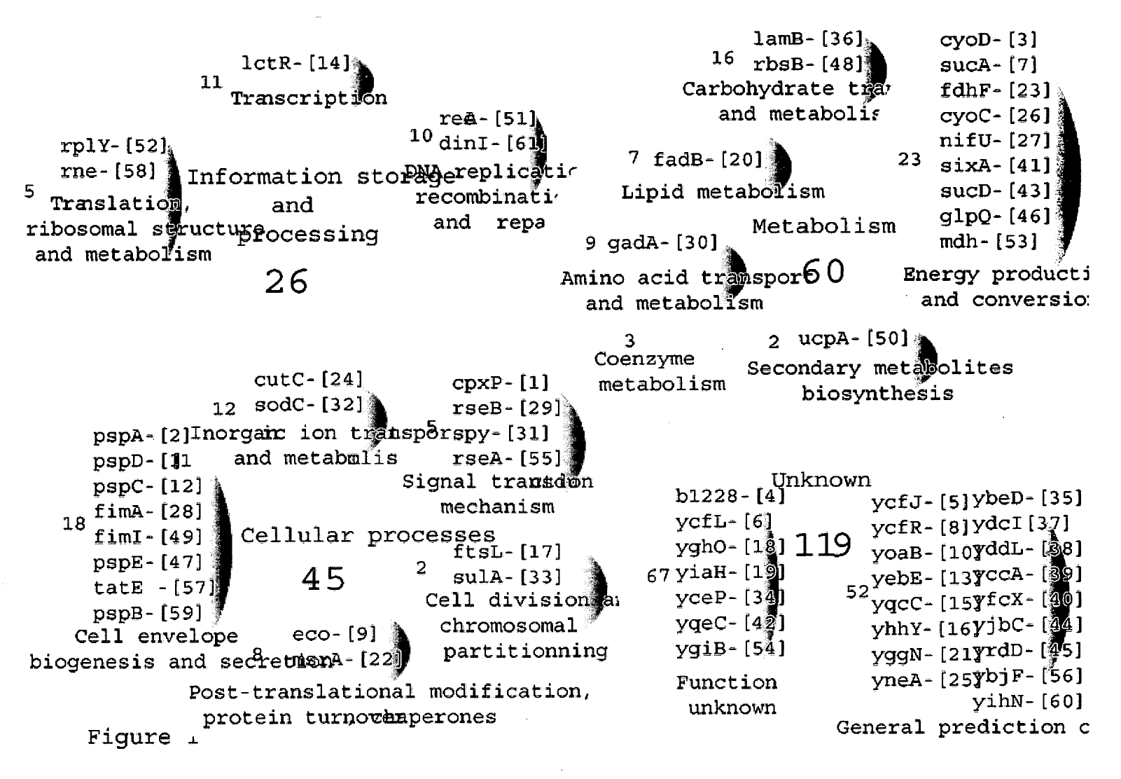

Total RNAs were isolated from independent biofilm and exponential growth phase cultures and subjected to a stringent expression profiling procedure using E. coli membrane DNA macroarrays. Data were subjected to a Wilcoxon rank test. The expression pattern and predicted function of differentially expressed genes are summarized in FIG. 1 and FIG. 7. In biofilms, 250 genes (5.8%) were over-expressed (p<0.05, 82% of them with p<0.005) whereas 188 genes (4.4%) were under-expressed (p<0.05, 85% of them with p<0.005). This indicates that 10.2% of the E. coli genome is differentially expressed in TG1 biofilm at a statistically significant level (FIGS. 1, 7 and Table 3 and 4). Among these identified genes, 1.9% were up or down-regulated by a factor of two-fold or more.

The most significant classes of biofilm-induced genes when compared to the planktonic exponential growth phase either by level of over-expression or by number are i) genes involved in cellular processes such as envelope stress-responses (pspABCDE, cpxP, spy, rpoE, rseA, rseB) and stress (recA, dinI) as well as cell envelope biogenesis and transport (fimA, tatE), ii) genes involved in energy (cyoD, sucA, sixA, nifU) and carbohydrate metabolic functions (rbsB, lamB) and iii) genes of unknown function (48%) (FIG. 1).

The main classes of repressed genes include genes involved in amino acid, carbohydrate transport and inorganic ion transport and genes of unknown function (FIG. 7 and Table 4). In the rest of this study, we focus on genes that were found to be the most over-expressed in E. coli biofilms. The role and significance of the repressed functions will be reported elsewhere.

Both Stationary Phase and Biofilm-Specific Genes Are Expressed in Mature Biofilms

Mature biofilms constitute heterogeneous environments where bacteria grow at different rates. This heterogeneity is proposed to be mostly dependent on nutrient availability and depth-related conditions created within the biofilm. We wished to determine to what extent the genes identified above were truly biofilm-specific or, instead, a consequence of the stationary phase-like conditions prevailing in the mature biofilm. Total RNAs were isolated from independent stationary phase planktonic cultures, subjected to the expression profiling procedure and compared to biofilm profiling (complete comparison is published). Among the 64 genes found to be the most induced in biofilm versus exponential phase (≧two-fold ratio, see FIG. 1), 61% (39/64) of them were not induced in biofilm when compared to stationary phase (Table 1). This suggests that these 39 genes are not biofilm-specific, but may, instead, reflect the stationary phase-like growth conditions within the mature E. coli biofilm.

In contrast, 39% (25/64) of the remaining genes were also over-expressed in biofilm versus stationary growth phase, 24 of which with a ratio ≧2, thus defining a set of biofilm-specific genes (Table 1 and Table 5).

Validation of the Macroarray Data

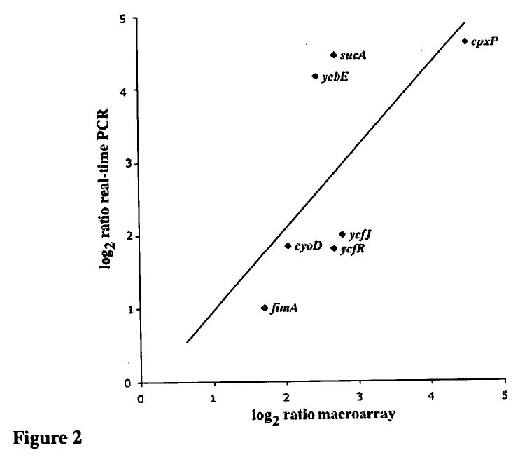

Several approaches were used to validate the data issued from transcriptional profiling experiments. We checked the correlation between expression data and operons structure in E. coli. An analysis restricted to the genes with known function found to be induced by at least a two-fold factor in biofilm compared to exponentially grown cells showed that 51% of them (21/41) were predicted to be included in 14 different operons, using the EcoCyc Database. For 10 of these 14 operons, we identified at least two members of the operon whose expression was induced in biofilms compared to exponentially grown cells. Furthermore, in order to verify the expression level changes, we then performed a Quantitative RT-PCR analysis (Q-RT-PCR) on a selection of the biofilm growth-regulated genes. Q-RT-PCR was performed for 7 of the most biofilm-induced genes compared to exponentially grown cells (cpxP, ycfJ, ycfR, yebE, cyoD, sucA and fimA, see FIG. 1 and Table 1). FIG. 2 shows a good correlation between the data obtained by the two different techniques (r=1.12).

These results indicate both a good internal consistency of our macroarray data as well as a good correlation between our analysis and actual mRNA level, as experimentally determined by Q-RT-PCR. To extract further functional information from our DNA-array data, we then wished to analyze the biofilm-related phenotypes of isogenic mutants of the identified biofilm-induced genes.

Functional Profiling of E. coli Biofilms: 20 Biofilm-Induced Genes Are Involved in Mature Biofilm Development

Among genes significantly induced in TG1 biofilms (when compared to planktonic exponential growth phase cells), 64 genes were found to be over-expressed by at least a factor of two (Table 1). To test directly the contribution of these genes to biofilm development, we deleted 23 of the 25 genes that were over-expressed in biofilms compared to both planktonic phases (biofilm-specific genes) as well as 31 of the 39 genes that were only induced in biofilms versus exponential growth phase. Mutations in sixA, sucA, yfhN (nifU), yfhO (nifS), ybeD, yhhY, rpoE and rseA impaired growth in M63B1 glucose minimal medium (data not shown). Mutants in these genes, along with ftsL, an essential cell division gene, could not be meaningfully tested for biofilm formation and were therefore excluded from further biofilm analysis. rpoE is an essential gene which mutations can be suppressed by extragenic mutations (De Las Penas et al., 1997, J Bacteriol 179: 6862-6864). Although our rpoE mutant did not exhibit full wild-type growth, we cannot exclude the appearance of such suppressor mutations in this mutant.

The ability to form a mature biofilm within 24 hours was assessed for each mutant and compared to TG1. Both macroscopic biofilm development in micro-fermenters and biofilm cell density after dispersion of the biofilm grown on the removable glass slide of the fermenter were examined. Twenty mutants displayed a reduced biofilm phenotype (see Table 1, FIG. 3 and FIG. 8). Nine of the mutants with reduced biofilm biomass correspond to genes of known function: fimA, msrA, rbsB, mdh, lctR, tatE, recA, cpxP and spy.

fimA, msrA, rbsB and mdh are genes encoding proteins that have been already linked to biofilm formation or adhesion properties (see above). As expected, adhesion appeared to be a key factor of TG1 biofilm formation. Indeed, fimA encodes for the major subunit of type I fimbriae, a known initial adhesion factor (Klemm and Christiansen, 1987, Mol Gen Genet 208: 439-445) whose role has been previously demonstrated in biofilm formation (Austin et al., 1998, FEMS Microbiol Lett 162: 295-301; Cookson et al., 2002, Int J Med Microbiol 292: 195-205; Cormio et al., 1996, Scand J Urol Nephrol 30: 19-24; Pratt and Kolter, 1998, Mol Microbiol 30: 285-293; Watnick et al., 1999, J Bacteriol 181: 3606-3609). In contrast with our results, Reisner et al. recently showed that a fimA mutation had no effect on the development of biofilms formed in flow chambers by a F plasmid-bearing E. coli strain (Reisner et al., 2003, Mol Microbiol 48: 933-946). Differences in strain, medium and biofilm growing system used might account for this discrepancy. msrA encodes a peptide methionine sulfoxide reductase (MsrA), a repair enzyme, that contributes to the maintenance of adhesins in Streptococcus pneumoniae, Neisseria gonorrhoeae, E. coli (Wizemann et al., 1996, Proc Natl Acad Sci USA 93: 7985-7990) and in Mycoplasma genitalium (Dhandayuthapani et al., 2001, J Bacteriol 183: 5645-5650), which could explain the alteration of biofilm formation in the msrA mutant.

The biofilm lifestyle leads to a profound modification of energy metabolism as judged by the identification of mdh, rbsB and lctR as biofilm-induced genes. The rbsB and mdh genes have been already identified as being over-expressed in biofilms formed by pathogenic E. coli (Tremoulet et al., 2002, FEMS Microbiol Lett 215: 7-14). rbsB is part of the rbsDACBK operon that encodes high affinity transport of and chemotaxis towards D-ribose (rbsC and rbsD are also induced in biofilm, see Table 3). mdh encodes malate dehydrogenase, an enzyme of the TCA cycle. The lctR gene encodes for a regulator of L-Lactate dehydrogenase. Furthermore, several sugar metabolism/transport systems are activated in biofilm (maltose transport, glycerol metabolism and uptake, galactose binding proteins, see Table 3).

Our results also suggest that mature E. coli biofilm formation might require Tat-dependent secretion of a specific set of proteins. Indeed, tatE is proposed to be involved in the twin-arginine cell envelope protein transport system (Chanal et al., 1998, Mol Microbiol 30: 674-676). In P. aeruginosa, tatA and tatB, encoding components of this secretion system, have been shown to be induced in biofilms (Whiteley et al., 2001, Nature 413: 860-864), whereas tatC have been shown to be required for biofilm formation (Ochsner et al., 2002, Proc Natl Acad Sci USA 99: 8312-8317).

We also observed a defect in mature biofilm formation in a recA mutant (FIG. 3). This underlines the importance of stress-responses in E. coli TG1 biofilm. Consistent with this result, several stress-response genes are over-expressed in TG1 biofilm (SOS response: dinI, dinP, dinG, sbmC, recN, sulA; general stress: rpoS; chaperones: dnaJ and dnaK; heat-shock proteins: htpX, htpG and ddg; DNA repair: exo, xthA and envelope stress: see Table 3 and below). cpxP and spy are both linked to envelope stress response (Connolly et al., 1997, Genes Dev 11: 2012-2021; Danese and Silhavy, 1997, Genes Dev 11: 1183-1193; Raivio and Silhavy, 2001, Annu Rev Microbiol 55: 591-624) and will be investigated below.

We could also assign a biofilm-related function to 11 genes of previously unknown function (ycfJ, ycfR, yoaB, yqcC, yggN, yneA, yccA, yfcX, yghO, yceP and ygiB). YfcX may be required for fatty acid utilization as a carbon source in anaerobic conditions (Campbell et al., 2003, Mol Microbiol 47: 793-805). Among these 11 genes, 5 encode putative extra-cytoplasmic proteins (ycfJ, ycfR, yqcC, yneA, yccA). YcfJ is homologous to UmoD of P. mirabilis, a protein that negatively regulates the flhDC flagellar and swarming master operon (Dufour et al., 1998, Mol Microbiol 29: 741-751). yccA is a putative cpx-regulon member (De Wulf et al., 2002, J Biol Chem 277: 26652-26661) encoding a protein of unknown function but it has been shown to be a substrate for the membrane protease FtsH (Kihara et al., 1998, J Mol Biol 279: 175-188). Among the mutants lacking any one of these five putative extra-cytoplasmic proteins, ΔycfJ and ΔyccA were the most affected for mature biofilm formation, with a reduction of about 50% compared to wild type strain TG1 (FIG. 8).

To investigate the biofilm-related role of these two putative membrane proteins further and to confirm their importance in mature biofilm formation, we genetically introduced the Green Fluorescent Protein (GFP) gene into the wild type strain TG1, and in the mutant strains TG1ΔycfJ and TG1ΔyccA. This allowed us to compare biofilm formation between TG1gfp and TG1gfpΔycfJ and TG1gfpΔyccA in continuous flow chamber cultures, another well established experimental model that is a non-invasive means of observing where the spatial arrangement of the cells is preserved. This experimental system allows the quantitative, real-time monitoring of biofilm architecture development using Confocal Laser Scanning Microscopy and COMSTAT analysis (Heydorn et al., 2000, Microbiology 146 (Pt 10): 2395-2407) (FIG. 4). Initial adhesion of the two ycfJ and yccA mutants was not affected, as measured by substrate coverage and biomass analysis. However, the maturation of the biofilm formed by these two mutants was strongly delayed, especially for the yccA mutant. Indeed, in the yccA mutant, the accumulated biomass remained very low over time and typical biofilm mushroom structures appeared only sporadically and much later compared to wild type strain TG1 (see FIG. 4). This suggests a role of YcfJ and YccA proteins in biofilm maturation.

These results demonstrate the involvement in mature biofilm formation of 30% of the most highly expressed genes identified in our study. 50% of these genes (10/20) were induced in biofilm versus both exponential and stationary growth phase (cpxP, spy, tatE, lctR, mdh, rbsB, ygiB, yqcC, yceP and yfcX) whereas the other 50% (10/20) were only induced in biofilm versus exponential growth phase (fimA, msrA, recA, yoaB, ycfJ, ycfR, yneA, yccA, yggN and yghO) (see Table 1 and Table 5).

Biofilm-Induced Genes Are Not Involved in the Early Stage of Biofilm Formation

A failure to form a wild type mature biofilm could result from an initial adhesion defect. Therefore, we investigated whether the genes identified as over-expressed in mature TG1 biofilms and that impaired mature biofilm formation when mutated were also involved in the early adhesion steps. For this, we tested this mutants in a static microtiter plate-based assay that has been widely used to study the first steps of biofilm formation (Genevaux et al., 1996, FEMS Microbiol Lett 142: 27-30; O'Toole et al., 1999, Methods Enzymol 310: 91-109). With the exception of fimA, the early adhesion capacity of the mutants could not be distinguished from the parental strain (FIG. 9). This result indicates that most genes over-expressed in mature biofilms are not involved in the early steps of this process and confirms that they participate in mature biofilm functions.

Comparison of E. coli F⁺/F⁻ Biofilm Global Response: General Relevance to E. coli Biofilm

In this study, we used an E. coli strain carrying a conjugative plasmid, a widespread situation which promotes biofilm formation (Ghigo, 2001, Nature 412: 442-445; Reisner et al., 2003., Mol Microbiol 48: 933-946). To distinguish general features of E. coli biofilms from those specific to our model, we analyzed the transcription profile of the E. coli strain TG, an F-free isogenic derivative of TG1. This control is of particular relevance because some of the genes found to be the most over-expressed (pspA, cpxP) have either been shown to be related to the conjugation process (cpx stands for conjugation plasmid expression (McEwen and Silverman, 1980, Proc Natl Acad Sci USA 77: 513-517) or to stress-responses that could correlate with the expression of membrane appendages such as conjugative pili. TG forms a thin and fragile biofilm after 5 days of culture in micro-fermenters (data not shown). Total RNA was isolated from E. coli TG biofilm and flask planktonic exponential cultures, and was subjected to the same macroarray analysis as described for TG1. TG1 and TG biofilms were not strictly comparable in terms of depth and structure (and therefore, possibly, for biofilm-induced responses). As expected, some functions induced in TG1, for instance RecA and part of the SOS stress pathway, were not induced in TG (Table 1), suggesting that F-specific, possibly transfer-related, responses are induced in TG1 biofilm. Despite this fact, 33% of the genes induced in TG1 biofilm by an over two-fold factor were also found to be statistically significantly over-expressed in TG biofilm (including cpxP, rseA, rseB, spy, psp operon members, tatE, and fimA, see Table 1). This demonstrates that many of the biofilm-induced genes identified in this study are F-independent and part of a general E. coli K12 biofilm response.

Envelope Stress Pathways in E. coli Mature Biofilm

cpxP is one the most over-expressed genes in E. coli TG1 biofilms versus planktonic growth phase (FIG. 1, Table 1 and Table 5). cpxP is a target of the cpx two-component system, which is known to respond to a variety of extra-cytoplasmic stress (envelope stress) (Raivio and Silhavy, 2001, Annu Rev Microbiol 55: 591-624).

We therefore investigated the effect of deletion mutations in key components of the cpx pathway on biofilm formation. As shown in FIG. 5, inactivation of the sensor-regulator components of the cpx system (cpxA, cpxR), but also of cpxP and of nlpE affected biofilm formation in micro-fermenters. A mutation in spy (a biofilm-induced cpxP homolog) has no effect on biofilm biomass. rpoE and rseA mutants displayed a growth rate defect and consequently could not be studied in micro-fermenters. A mutation in rseB, the second anti-sigma E factor of the RpoE envelope stress pathway, did not affect growth and a rseB mutant formed a wild type biofilm. Whereas it is difficult to conclude that the rpoE pathway has a role in biofilm formation, the cpx pathway appears to contribute to biofilm development, based on the morphological effects caused by mutations in several of its key components. Indeed, the biofilms produced by both TG1ΔcpxR and TG1ΔcpxP in micro-fermenters were very fragile compared to wild type TG1 biofilms. TG1ΔcpxP biofilm was made of large plaques, in strong contrast to the homogeneous TG1 biofilm (FIG. 6ABC). Consistent with this observation, a detailed electron microscopy analysis revealed that a cpxP mutation strongly altered biofilm macromorphology (FIG. 6DE). Despite its fragility, no clear structural defect could be detected in the TG1ΔcpxR biofilm (data not shown). Even though slight structural differences could also be seen in the TG1Δspy mutant biofilms, structural alterations were not found in nlpE, cpxA nor rseB mutant biofilms grown in micro-fermenters (data not shown).

To further investigate the role of cpxP and cpxR, we introduced a gfp allele into TG1ΔcpxP and TG1ΔcpxR and we compared their biofilm formation to the parental TG1gfp strain in continuous flow chamber cultures. Single cells and very small colonies were observed on the surface for these two mutants during the initial steps of biofilm development in contrast to the wild-type that forms normal three-dimensional colonies (FIG. 4, 20 and 45 h). Furthermore, both cpxP and cpxR mutants were also strongly affected for maturation of the biofilm (FIG. 4). These experiments suggest that stress envelope pathways are involved in the establishment of a structured mature biofilm in E. coli.

Phage-shock protein operon (psp) is expressed in response to a variety of environmental and intracellular stresses including processes related to protein insertion in the outer membrane (Weiner and Model, 1994, Proc Natl Acad Sci USA 91: 2191-2195). While the precise functions of the psp genes are not understood, they help to ensure survival of E. coli in adverse conditions, suggesting that psp genes are part of a stress-response operon (Model et al., 1997, Mol Microbiol 24: 255-261). In our analysis, pspA and other members of the operon (pspBCDE) were consistently over-expressed in biofilm (FIG. 1, Table 3 and Table 5). Nevertheless, the disruption of the pspABCDE operon did not have a major impact on early (FIG. 9) or late biofilm formation nor on biofilm structure (data not shown).

### Discussion

In this study we investigated the differences in gene expression between E. coli K12 mature biofilm and planktonic laboratory cultures. Using DNA macroarrays we showed that the biofilm lifestyle, while sharing similarities with the stationary growth phase, triggers the expression of specific sets of genes.

Modifications of E. coli K12 Gene Expression Induce by the Biofilm Lifestyle

The use of large scale fusion technology had already suggested that a significant fraction of the bacterial genome could be involved in biofilm physiology (Prigent-Combaret et al., 1999, J Bacteriol 181: 5993-6002). Accordingly, P. putida and P. aeruginosa biofilm proteome analyses showed that a large number of genes are differentially regulated during biofilm development (Sauer and Camper, 2001, J Bacteriol 183: 6579-6589; Sauer et al., 2002, J Bacteriol 184: 1140-1154). In contrast, a transciption profiling of the P. aeruginosa planktonic and biofilm phases led to the conclusion that only 1% of P. aeruginosa genes display over a two-fold difference in gene expression (Whiteley et al., 2001, Nature 413: 860-864).

In E. coli, Schembri et al. recently showed that approximately 5 to 10% of the E. coli genes exhibited altered microarray expression profiles when compared planktonic growth phases and young biofilm cultures. They hypothesized that this could be due to the rather early stages of biofilm development analyzed in their study, where the still ongoing switch from planktonic to sessile growth could result in a high level of transient gene expression (Schembri et al., 2003, Mol Microbiol 48: 253-267).

Here, we compared mature biofilms to the planktonic exponential growth phase and showed that, as in the case of mature P. aeruginosa biofilms, only a small fraction (1.9%) of the E. coli genes are differentially expressed by more than a factor of two. However, below that threshold, biofilm formation still leads to the statistically significant differential expression of more than 10% of the E. coli genome. These results therefore support the proposal that biofilm formation results in and from significant differences in the overall make-up of bacterial cells (Sauer, 2003, Genome Biol 4: 219; Stoodley et al., 2002, Annu Rev Microbiol 56: 187-209).

Mature biofilm cells have been proposed to have stationary growth phase traits such as reduced growth and metabolic activity. To investigate the stationary phase character of bacterial life within biofilm, we also compared the expression pattern of stationary phase cultures with those determined for the exponential growth phase and the mature biofilm. Biofilm-specific genes, i.e. genes differentially regulated in biofilm versus both forms of planktonic phases, correspond to 4% of the genome (118 over- and 53 under-expressed/4290) and this proportion decreases to less than 1% (0.67%, 23 over and 6 under/4290) for genes varying by a factor of more than two. When one only considers the genes induced in response to the stationary growth phase character of the biofilm lifestyle, these genes represent 3% of the genome. The biofilm lifestyle, while sharing similarities with the stationary growth phase, thus triggers the expression of specific sets of genes.

Functional Profiling of the Biofilm-Induced Genes

The biological importance of the differential gene expression exhibited upon biofilm versus planktonic growth was tested by the disruption of the majority of the highly-induced genes in biofilms, including all biofilm-specific induced genes. We show that, while the mutants were not impaired in initial steps of adhesion to surfaces (with the exception of fimA), a third of them (20 genes) were affected in the biofilm maturation (Table 1, FIG. 3 and FIG. 8). This high proportion of genes involved in the biofilm maturation strongly supports the pertinence of our analysis. Among these 20 genes, half correspond to biofilm-specific genes whereas the other half was only induced in biofilms versus exponential growth phase (see Table 1 and Table 5). This indicates that the development of a full mature biofilm requires not only biofilm-specific genes but also genes related to the stationary phase character of the biofilm. The individual role of some of these newly identified genes is currently being investigated.

Biofilm-Related Physiological Functions

We show that genes found to be the most over-expressed in TG1 biofilm versus exponential growth phase were also part of the E. coli F-free biofilm response, therefore indicating that genes identified in this study are involved in the general response developed in mature E. coli K12 biofilms. Those genes are not distributed randomly into all potential functional classes. Instead they display a strong bias toward specific functional categories and we propose that they are part of the biofilm genetic signature. Genes whose expression is required for full maturation of TG1 biofilm belong to functions linked to adhesion (fimA, msrA), energy metabolism (rbsB, mdh, lctR), transport (tatE), general stress (recA), and envelope stress response (cpxP and spy). However, it is likely that many genes identified in our study are not specifically involved in biofilm-specific functions but rather correspond to adaptive responses to the biofilm environment. Mutations in many biofilm-induced genes that also correspond to information storage and processing, metabolism, cellular processes and unknown functions have indeed no effect on TG1 biofilm formation (Table 1).

Moreover, 48% of the genes significantly over-expressed in biofilms versus exponential growth phase were of uncharacterized function. Compared to 19.6% of such genes found in the E. coli genome (Serres et al., 2001, Genome Biol 2: RESEARCH0035), this high proportion of genes of unknown functions expressed in mature biofilm suggests that new aspects of E. coli biology are adopted during biofilm formation. We show that 11 of these uncharacterized genes are necessary for full mature biofilm formation, thus experimentally assigning them a biofilm-related function (Table 1, FIG. 3 and FIG. 8). Among them 5 encode putative membrane proteins that could be of particular relevance when considering the importance of envelope-related physiology within a biofilm.

Consistent with the drastic phenotypic changes occurring inside biofilms, we found that 15% of the genes identified as over- or under-expressed in biofilms versus exponential growth phase are involved in either energy processes or carbohydrate metabolism (FIG. 1, Table 1 and 3). Despite the presence of polysaccharides in the TG1 biofilm (data not shown), we could not clearly associate the expression of any of those genes with the production of the biofilm matrix (i.e., cellulose, colanic acid). This could reflect, among other explanations, a lack of sensitivity of our approach due to the averaging occurring while extracting transcription information from the heterogeneous bacterial biofilm population.

A partial comparison of the most over-expressed genes in our analysis (>2 fold factor) and in the study by Schembri et al. (>8 fold factor) only revealed a few genes identified as over-expressed in E. coli biofilm in both studies (rbsB, b0836, yfjO, yceP, glgS, ydeW, yneA, yqeC, ylcC, rplV, rplD, rpsS, b1550, rplP, rpsR, flu, rplM, ppc, oppA, gatD, cydA, atpB, rpsN, malK, atpG) (Schembri et al., 2003, Mol Microbiol 48: 253-267). Three of these genes (rbsB, yceP, yneA) were nevertheless also found here to be required for mature biofilm formation. This relatively low overlap between the two studies may be due to technical differences. Different scenarios were used in terms of strain background, media and experimental set-up. This could also reflect the difference in the gene expression pattern between two biofilms at very different stages of maturation (i.e. young and thin biofilms in Schembri et al. versus mature and thick biofilms in our study). Further studies comparing the expression profile of E. coli biofilms at different maturation stages within the same experimental set-up will provide a more dynamic view of biofilm gene expression.

Heterogeneity of Oxygen Conditions in E. coli K12 Biofilms

Biofilms are heterogeneous environments and, with respect to aerobiosis, our analysis supports these results. In the main experiment described in this study, we compared exponentially grown agitated flask cultures to TG1 biofilm in aerated conditions. Under these conditions, numerous genes known to be induced by aerobiosis were also induced in biofilms, including some genes for TCA cycle enzymes (e.g. aceB, cyo operon members, fadB, mdh, glpD, sucAB). In addition, some genes known to be repressed by aerobiosis were repressed in biofilms (eg. adhE, cydAB, dcuC, focA, fumB). This tends to indicate that our biofilms were mainly grown under aerobic conditions. Consequently, we also compared differential gene expression between TG1 biofilms and TG1 planktonic cultures, both grown in aerated fermenters (data not shown). In this configuration, we clearly observed that some typical aerobic genes were induced in biofilms whereas others were repressed. This was also the case for typical anaerobic genes. This could reflect the heterogeneity of the aerobic conditions in biofilms, in which external bacteria are in contact with oxygen while internal bacteria are in conditions close to anaerobiosis.

Stress-Responses in Biofilms

Our study revealed that a major physiological response to biofilm formation is the induction of stress-responses. Interestingly, such a stress-response induction may also take place in P. aeruginosa biofilms. Indeed, the most highly activated genes identified in a P. aeruginosa biofilm transcriptome analysis were those of temperate bacteriophages (Whiteley et al., 2001, Nature 413: 860-864). As stresses are known to induce prophages and other mobile genetic elements, our results suggest that Pseudomonas prophage induction may be a consequence of stresses created by the drastic conditions that prevail inside the biofilm. As such, stress may well be a key factor in the mechanisms that lead to the observed antibiotic resistance inside biofilm communities.

Owing to the possible role of cell-cell and cell-surface interactions in biofilm, it may be of significance that envelope stress genes such as cpxP, spy and the psp genes are consistently induced in this environment. CpxP may inhibit the cpx-mediated induction through a direct interaction with the two-component system sensor CpxA, while Spy may play a similar role on the rpoE pathway (Raivio et al., 2000, Mol Microbiol 37: 1186-1197). The cpx system is known to respond to envelope stresses such as over-production and misfolding of membrane proteins or elevated pH (Raivio and Silhavy, 2001, Annu Rev Microbiol 55: 591-624). However, relatively little is known about the physiological role of envelope stress-responses. Recently, adhesion of E. coli cells to hydrophobic but not hydrophilic surfaces was shown to activate the cpx system, including cpxP, through a process called surface sensing which requires both cpxR and nlpE (Otto and Silhavy, 2002, Proc Natl Acad Sci USA 99: 2287-2292). Consistently, we find that cpxP and spy are highly induced in mature biofilms where bacteria are de facto in contact with the hydrophobic surfaces of other cells.

Our results thus provide additional experimental evidence that stress response pathways are key factors in biofilm formation. The structure of biofilms grown in micro-fermenters is altered in a cpxP mutant (FIG. 6) and to a lesser extent in a spy mutant. Observation of spy mutant biofilms by transmission electron microscopy also revealed a high proportion of spheroblasts as compared to wt TG1 (data not shown), suggesting a possible cause for the affected structure of the biofilm in this mutant. In addition, a cpxP and a cpxR mutant are both impaired in forming wild type micro-colonies (FIG. 4). This strongly corroborates the idea that cpxP and cpxR mutants have reduced cell-to-cell adherence, since any growth up in the water column will be counteracted by the shearing forces of the flow. It appears, then, that the inappropriate expression of the cpx regulated genes in biofilm, i.e. a derepression of the cpx regulon in the cpxP mutant or an absence of induction of the cpx regulon in the cpxR mutant, leads to an alteration of the process of biofilm formation. Considering the importance of environmental conditions in biofilm formation, two component systems, which sense perturbations or changes in the bacterial environment, might play a regulatory role in bacterial biofilm formation, a proposal that requires further investigation.

Our analysis identified the biofilm mode of growth as an environment that induces the expression of the pspABCDE stress operon. However no biofilm-related phenotype could be observed in a strain deleted for the pspABCDE operon. Nevertheless, the deletion of pspF, a constitutively expressed positive regulator of the pspABCDE operon, affects biofilm formation (FIG. 8). Since pspABCDE is not required for biofilm formation, pspF might also regulate a biofilm-related locus that is not part of pspABCDE operon. Evidence for such an additional PspF regulated target has been provided in the case of Yersinia enterolitica psp regulon (Darwin and Miller, 2001, Mol Microbiol 39: 429-444).

Changes in Gene Expression and Biofilm Development

The changes in gene expression demonstrated here and in other studies could be considered either as part of the E. coli biofilm development (needed for maturation) or as caused by the conditions progressively created within the biofilm during its maturation (consequence of the maturation). The first hypothesis implies that the biofilm formation is a developmental process in which genetic checkpoints could control the maturation of the biofilm by inducing a succession of biofilm-specific genes. Whereas 8 mutations out of 54 mutants created in this study display a 50% decrease in biofilm biomass and maturation, none of them lead to a total loss of biofilm formation. Considering the existence of multiple and partially overlapping or complementing pathways that can lead to biofilm formation, this result, without formally excluding the existence of a biofilm developmental program, rather speaks in favor of the second working hypothesis. In this case, most changes observed in biofilm gene induction could be a consequence of, rather than a prerequisite for the biofilm maturation.

The results presented here provide new insights into the global effect triggered by biofilm formation in E. coli. By monitoring the changes in gene expression occurring in mature biofilms, we have identified biofilm-related physiological pathways and previously uncharacterized biofilm-induced genes. This may lead to new biofilm control strategies that will likely hinge upon a better understanding of biofilm-induced physiological responses.

### Discussion

In this study we investigated the differences in gene expression between E. coli K12 mature biofilm and planktonic laboratory cultures. Using DNA macroarrays we showed that the biofilm lifestyle, while sharing similarities with the stationary growth phase, triggers the expression of specific sets of genes.

Modifications of E. coli K12 Gene Expression Induce by the Biofilm Lifestyle

The use of large scale fusion technology had already suggested that a significant fraction of the bacterial genome could be involved in biofilm physiology (Prigent-Combaret et al., 1999, J Bacteriol 181: 5993-6002). Accordingly, P. putida and P. aeruginosa biofilm proteome analyses showed that a large number of genes are differentially regulated during biofilm development (Sauer and Camper, 2001, J Bacteriol 183: 6579-6589; Sauer et al., 2002, J Bacteriol 184: 1140-1154). In contrast, a transciption profiling of the P. aeruginosa planktonic and biofilm phases led to the conclusion that only 1% of P. aeruginosa genes display over a two-fold difference in gene expression (Whiteley et al., 2001, Nature 413: 860-864).

In E. coli, Shembri et al. recently showed that approximately 5 to 10% of the E. coli genes exhibited altered microarray expression profiles when compared planktonic growth phases and young biofilm cultures. They hypothesized that this could be due to the rather early stages of biofilm development analyzed in their study, where the still ongoing switch from planktonic to sessile growth could result in a high level of transient gene expression (Schembri et al., 2003, Mol Microbiol 48: 253-267).

Here, we compared mature biofilms to the planktonic exponential growth phase and showed that, as in the case of mature P. aeruginosa biofilms, only a small fraction (1.9%) of the E. coli genes are differentially expressed by more than a factor of two. However, below that threshold, biofilm formation still leads to the statistically significant differential expression of more than 10% of the E. coli genome. These results therefore support the proposal that biofilm formation results in and from significant differences in the overall make-up of bacterial cells (Sauer, 2003, Genome Biol 4: 219; Stoodley et al., 2002, Annu Rev Microbiol 56: 187-209).

Mature biofilm cells have been proposed to have stationary growth phase traits such as reduced growth and metabolic activity. To investigate the stationary phase character of bacterial life within biofilm, we also compared the expression pattern of stationary phase cultures with those determined for the exponential growth phase and the mature biofilm. Biofilm-specific genes, i.e. genes differentially regulated in biofilm versus both forms of planktonic phases, correspond to 4% of the genome (118 over- and 53 under-expressed/4290) and this proportion decreases to less than 1% (0.67%, 23 over and 6 under/4290) for genes varying by a factor of more than two. When one only considers the genes induced in response to the stationary growth phase character of the biofilm lifestyle, these genes represent 3% of the genome. The biofilm lifestyle, while sharing similarities with the stationary growth phase, thus triggers the expression of specific sets of genes.

Functional Profiling of the Biofilm-Induced Genes

The biological importance of the differential gene expression exhibited upon biofilm versus planktonic growth was tested by the disruption of the majority of the highly-induced genes in biofilms, including all biofilm-specific induced genes. We show that, while the mutants were not impaired in initial steps of adhesion to surfaces (with the exception of fimA), a third of them (20 genes) were affected in the biofilm maturation (Table 1, FIG. 3 and FIG. 8). This high proportion of genes involved in the biofilm maturation strongly supports the pertinence of our analysis. Among these 20 genes, half correspond to biofilm-specific genes whereas the other half was only induced in biofilms versus exponential growth phase (see Table 1 and Table 5). This indicates that the development of a full mature biofilm requires not only biofilm-specific genes but also genes related to the stationary phase character of the biofilm. The individual role of some of these newly identified genes is currently being investigated.

Biofilm-Related Physiological Functions

We show that genes found to be the most over-expressed in TG1 biofilm versus exponential growth phase were also part of the E. coli F-free biofilm response, therefore indicating that genes identified in this study are involved in the general response developed in mature E. coli K12 biofilms. Those genes are not distributed randomly into all potential functional classes. Instead they display a strong bias toward specific functional categories and we propose that they are part of the biofilm genetic signature. Genes whose expression is required for full maturation of TG1 biofilm belong to functions linked to adhesion (fimA, msrA), energy metabolism (rbsB, mdh, lctR), transport (tatE), general stress (recA), and envelope stress response (cpxP and spy). However, it is likely that many genes identified in our study are not specifically involved in biofilm-specific functions but rather correspond to adaptive responses to the biofilm environment. Mutations in many biofilm-induced genes that also correspond to information storage and processing, metabolism, cellular processes and unknown functions have indeed no effect on TG1 biofilm formation (Table 1).

Moreover, 48% of the genes significantly over-expressed in biofilms versus exponential growth phase were of uncharacterized function. Compared to 19.6% of such genes found in the E. coli genome (Serres et al., 2001, Genome Biol 2: RESEARCH0035), this high proportion of genes of unknown functions expressed in mature biofilm suggests that new aspects of E. coli biology are adopted during biofilm formation. We show that 11 of these uncharacterized genes are necessary for full mature biofilm formation, thus experimentally assigning them a biofilm-related function (Table 1, FIG. 3 and FIG. 8). Among them 5 encode putative membrane proteins that could be of particular relevance when considering the importance of envelope-related physiology within a biofilm.

Consistent with the drastic phenotypic changes occurring inside biofilms, we found that 15% of the genes identified as over- or under-expressed in biofilms versus exponential growth phase are involved in either energy processes or carbohydrate metabolism (FIG. 1, Table 1 and 3). Despite the presence of polysaccharides in the TG1 biofilm (data not shown), we could not clearly associate the expression of any of those genes with the production of the biofilm matrix (i.e., cellulose, colanic acid). This could reflect, among other explanations, a lack of sensitivity of our approach due to the averaging occurring while extracting transcription information from the heterogeneous bacterial biofilm population.

A partial comparison of the most over-expressed genes in our analysis (>2 fold factor) and in the study by Schembri et al. (>8 fold factor) only revealed a few genes identified as over-expressed in E. coli biofilm in both studies (rbsB, b0836, yfjO, yceP, glgS, ydeW, yneA, yqeC, ylcC, rplV, rplD, rpsS, b1550, rplP, rpsR, flu, rplM, ppc, oppA, gatD, cydA, atpB, rpsN, malK, atpG) (Schembri et al., 2003, Mol Microbiol 48: 253-267). Three of these genes (rbsB, yceP, yneA) were nevertheless also found here to be required for mature biofilm formation. This relatively low overlap between the two studies may be due to technical differences. Different scenarios were used in terms of strain background, media and experimental set-up. This could also reflect the difference in the gene expression pattern between two biofilms at very different stages of maturation (i.e. young and thin biofilms in Schembri et al. versus mature and thick biofilms in our study). Further studies comparing the expression profile of E. coli biofilms at different maturation stages within the same experimental set-up will provide a more dynamic view of biofilm gene expression.

Heterogeneity of Oxygen Conditions in E. coli K12 Biofilms

Biofilms are heterogeneous environments and, with respect to aerobiosis, our analysis supports these results. In the main experiment described in this study, we compared exponentially grown agitated flask cultures to TG1 biofilm in aerated conditions. Under these conditions, numerous genes known to be induced by aerobiosis were also induced in biofilms, including some genes for TCA cycle enzymes (e.g. aceB, cyo operon members, fadB, mdh, glpD, sucAB). In addition, some genes known to be repressed by aerobiosis were repressed in biofilms (eg. adhE, cydAB, dcuC, focA, fumB). This tends to indicate that our biofilms were mainly grown under aerobic conditions. Consequently, we also compared differential gene expression between TG1 biofilms and TG1 planktonic cultures, both grown in aerated fermenters (data not shown). In this configuration, we clearly observed that some typical aerobic genes were induced in biofilms whereas others were repressed. This was also the case for typical anaerobic genes. This could reflect the heterogeneity of the aerobic conditions in biofilms, in which external bacteria are in contact with oxygen while internal bacteria are in conditions close to anaerobiosis.

Stress-Responses in Biofilms

Our study revealed that a major physiological response to biofilm formation is the induction of stress-responses. Interestingly, such a stress-response induction may also take place in P. aeruginosa biofilms. Indeed, the most highly activated genes identified in a P. aeruginosa biofilm transcriptome analysis were those of temperate bacteriophages (Whiteley et al., 2001, Nature 413: 860-864). As stresses are known to induce prophages and other mobile genetic elements, our results suggest that Pseudomonas prophage induction may be a consequence of stresses created by the drastic conditions that prevail inside the biofilm. As such, stress may well be a key factor in the mechanisms that lead to the observed antibiotic resistance inside biofilm communities.

Owing to the possible role of cell-cell and cell-surface interactions in biofilm, it may be of significance that envelope stress genes such as cpxP, spy and the psp genes are consistently induced in this environment. CpxP may inhibit the cpx-mediated induction through a direct interaction with the two-component system sensor CpxA, while Spy may play a similar role on the rpoE pathway (Raivio et al., 2000, Mol Microbiol 37: 1186-1197). The cpx system is known to respond to envelope stresses such as over-production and misfolding of membrane proteins or elevated pH (Raivio and Silhavy, 2001, Annu Rev Microbiol 55: 591-624). However, relatively little is known about the physiological role of envelope stress-responses. Recently, adhesion of E. coli cells to hydrophobic but not hydrophilic surfaces was shown to activate the cpx system, including cpxP, through a process called surface sensing which requires both cpxR and nlpE (Otto and Silhavy, 2002, Proc Natl Acad Sci USA 99: 2287-2292). Consistently, we find that cpxP and spy are highly induced in mature biofilms where bacteria are de facto in contact with the hydrophobic surfaces of other cells.

Our results thus provide additional experimental evidence that stress response pathways are key factors in biofilm formation. The structure of biofilms grown in micro-fermenters is altered in a cpxP mutant (FIG. 6) and to a lesser extent in a spy mutant. Observation of spy mutant biofilms by transmission electron microscopy also revealed a high proportion of spheroblasts as compared to wt TG1 (data not shown), suggesting a possible cause for the affected structure of the biofilm in this mutant. In addition, a cpxP and a cpxR mutant are both impaired in forming wild type micro-colonies (FIG. 4). This strongly corroborates the idea that cpxP and cpxR mutants have reduced cell-to-cell adherence, since any growth up in the water column will be counteracted by the shearing forces of the flow. It appears, then, that the inappropriate expression of the cpx regulated genes in biofilm, i.e. a derepression of the cpx regulon in the cpxP mutant or an absence of induction of the cpx regulon in the cpxR mutant, leads to an alteration of the process of biofilm formation. Considering the importance of environmental conditions in biofilm formation, two component systems, which sense perturbations or changes in the bacterial environment, might play a regulatory role in bacterial biofilm formation, a proposal that requires further investigation.

Our analysis identified the biofilm mode of growth as an environment that induces the expression of the pspABCDE stress operon. However no biofilm-related phenotype could be observed in a strain deleted for the pspABCDE operon. Nevertheless, the deletion of pspF, a constitutively expressed positive regulator of the pspABCDE operon, affects biofilm formation (FIG. 8). Since pspABCDE is not required for biofilm formation, pspF might also regulate a biofilm-related locus that is not part of pspABCDE operon. Evidence for such an additional PspF regulated target has been provided in the case of Yersinia enterolitica psp regulon (Darwin and Miller, 2001, Mol Microbiol 39: 429-444).

Changes in Gene Expression and Biofilm Development

The changes in gene expression demonstrated here and in other studies could be considered either as part of the E. coli biofilm development (needed for maturation) or as caused by the conditions progressively created within the biofilm during its maturation (consequence of the maturation). The first hypothesis implies that the biofilm formation is a developmental process in which genetic checkpoints could control the maturation of the biofilm by inducing a succession of biofilm-specific genes. Whereas 8 mutations out of 54 mutants created in this study display a 50% decrease in biofilm biomass and maturation, none of them lead to a total loss of biofilm formation. Considering the existence of multiple and partially overlapping or complementing pathways that can lead to biofilm formation, this result, without formally excluding the existence of a biofilm developmental program, rather speaks in favor of the second working hypothesis. In this case, most changes observed in biofilm gene induction could be a consequence of, rather than a prerequisite for the biofilm maturation.

The results presented here provide new insights into the global effect triggered by biofilm formation in E. coli. By monitoring the changes in gene expression occurring in mature biofilms, we have identified biofilm-related physiological pathways and previously uncharacterized biofilm-induced genes. This may lead to new biofilm control strategies that will likely hinge upon a better understanding of biofilm-induced physiological responses.

TABLE 1
Over-expressed genes (≧2) in E. coli TG1 and TG biofilms versus
exponential growth phase

| | | | | | | |
|---|---|---|---|---|---|---|
| Genes | | Ratio | Rank | Phenotype | TG | Function - description |
| a | b | c | d | e | f | g |

INFORMATION STORAGE AND PROCESSING
J: Translation, ribosomal structure and metabolism

| | | | | | |
|---|---|---|---|---|---|
| rplY* | b2185 | 2.23 | 52 | ND | 50S ribosomal subunit protein L25 |
| rne^{#} | b1084 | 2.06 | 58 | ND | RNase E, mRNA turnover, maturation 5S |
| | | | | | RNA |

K: Transcription

| | | | | | | |
|---|---|---|---|---|---|---|
| Ë | lctR^{#} | b3604 | 4.76 | 14 | — | Regulator of L-Lactate dehydrogenase |
| | | | | | | genes |

L: DNA replication, recombination and repair

| | | | | | | |
|---|---|---|---|---|---|---|
| Ë | recA* | b2699 | 2.30 | 51 | — | DNA strand exchange and renaturation. |
| | | | | | | SOS |
| | dinI^{#} | b1061 | 2.02 | 61 | wt | Inhibits RecA-mediated self-cleavage. SOS |

METABOLISM
C: Energy production and conversion

| | | | | | | | |
|---|---|---|---|---|---|---|---|
| | cyoD* | b0429 | 7.41 | 3 | wt | | Cytochrome o oxidase subunit IV |
| | sucA* | b0726 | 6.54 | 7 | NA | | 2-oxoglutarate dehydrogenase |
| | fdhF* | b4079 | 3.85 | 23 | wt | | S subunit of formate dehydrogenase H |
| | cyoC* | b0430 | 3.59 | 26 | wt | | Cytochrome o oxidase subunit III |
| | nifU* | b2529 | 3.41 | 27 | NA | | Formation of [fe-s] clusters in iron-sulfur |
| | | | | | | | proteins |
| | sixA^{#} | b2340 | 2.74 | 41 | wt | ✓ | Phosphatase affecting ArcB phosphorelay |
| | sucD* | b0729 | 2.69 | 43 | NA | | Succinyl-CoA synthetase, alpha subunit |
| | glpQ^{#} | b2239 | 2.50 | 46 | ND | | Glycerol-3-phosphate diesterase, |
| | | | | | | | periplasmic |
| Ë | mdh^{#} | b3236 | 2.19 | 53 | — | | Malate dehydrogenase |

G: Carbohydrate transport and metabolism

| | | | | | | |
|---|---|---|---|---|---|---|
| | lamB^{#} | b4036 | 2.94 | 36 | wt | Phage lambda receptor, maltose receptor |
| Ë | rbsB^{#} | b3751 | 2.41 | 48 | — | D-ribose periplasmic binding protein, |
| | | | | | | chemotaxis |
| | sfsA* | b0146 | 1.97 | 64 | ND | Regulatory protein for maltose metabolism |

E: Amino acid transport and metabolism

| | | | | | | |
|---|---|---|---|---|---|---|
| gadA^{#} | b3517 | 3.15 | 30 | wt | ✓ | Glutamate decarboxylase isozyme |
| nifS* | b2530 | 1.98 | 62 | NA | | Cysteine desulfurase |

I: Lipid metabolism

| | | | | | |
|---|---|---|---|---|---|
| fadB* | b3846 | 4.18 | 20 | wt | Fatty acid oxidation complex; 4-enzyme |
| | | | | | protein |

Q: Secondary metabolites biosynthesis, transport and metabolism

| | | | | | |
|---|---|---|---|---|---|
| ucpA* | b2426 | 2.32 | 50 | ND | Oxido reductase, dehydrogenase/reductase |
| | | | | | family |

CELLULAR PROCESSES
D: Cell division and chromosomal partitionning

| | | | | | | |
|---|---|---|---|---|---|---|
| ftsL* | b0083 | 4.34 | 17 | NA | | Cell division and growth, septum |
| | | | | | | localization |
| sulA* | b0958 | 3.07 | 33 | wt | 47 | Inhibits cell division. SOS |

O: Post-translational modification, protein turnover, chaperones

| | | | | | |
|---|---|---|---|---|---|
| eco* | b2209 | 6.17 | 9 | wt | Ecotin, a periplasmic serine protease |
| | | | | | inhibitor |

a: Gene names according to E. coli Colibri database.
b: Gene names according to Blattner nomenclature.
c: Ratio of gene expression in E. coli biofilm versus gene expression in planktonic cultures.
d: Rank position; 1 = the most over-expressed gene in E. coli biofilm.
e: Biofilm phenotype of the mutants:
ND: not determined;
NA: not applicable due to growth defect in M63B1 glucose medium;
wt: similar to wild type;
—: biofilm reduced compared to wt;
Struct: biofilm structure impaired compared to wt.
f: ✓, genes also found to be significantly over-expressed in F minus E. coli strain TG.
g: Function description according to E. coli Colibri database.
h: pspF was expressed by only a 1.22 factor in TG1 biofilm but has been included for comparison with other members of the psp operon.
Arrow: mutants affected for biofilm formation.
*genes that were not induced in TG1 biofilm versus stationary phase.
^{#}genes that were also induced in TG1 biofilm versus stationary phase by at least a factor of two. These genes are also summarized in Table 3S.
The genes have been classified according to the COGs functional categories annotation system used by the NCBI.

TABLE 2
Strains and plasmids used in this study
*Additional individual mutants in the following genes: cutC, cyoC, dinI, eco,
fadB, fdhF, gadA, lctR, malM-G, mdh, nifS, nifU, nlpE, pspA-E, rbsB, rpoE,
rseB, sixA, sodC, spy, sucA, sulA, tatE, ybeD, ybjF, yccA, yceP, ycfJ, ycfL,
ycfR, ydcI, yebE, yfcX, yggN, yghO, ygiB, yhhY, yiaH, yjbO, yneA, yoaB,
yqcC, yqeC, were named TG1Δ□gene.name]::aphA, (Km^{R}).

| Strain/plasmid | Relevant characteristics | Reference/source |
|---|---|---|
| E. coli strains | | |
| PAP6181 | K1519pspF::miniTn10 (Tet^{R}) | (Jovanovic et al., 1996) |
| PHL904 | cpxA:: Ωcat(Cm^{R}) | (Dorel et al., 1999) |
| RG075 | MG1655ΔmsrA::ΩSpec (Spec^{R}) | A gift of F. Barras |
| STC27 | fimA1::cat (Cm^{R}) | (Pratt and Kolter, 1998) |
| TG1 | F′[traD36 proAB+ lacI^{q} lacZΔM15] supE | Laboratory collection |
| | hsdΔ5 thi Δ(lac-proAB) | |
| TG | A F minus derivative of TG1 | Laboratory collection |
| TG1ΔcpxA | TG1cpxA:: Ωcat(Cm^{R}) | This work |
| TG1ΔcpxP | TG1ΔcpxP::Δfrt | This work |
| TG1ΔcpxR | TG1ΔcpxR::Δfrt | This work |
| TG1ΔfimA | TG1ΔfimA::cat (Cm^{R}) | This work |
| TG1ΔmsrA | TG1ΔmsrA::ΩSpec, (Spec^{R}) | This work |
| TG1ΔpspF | TG1pspF::miniTn10 (Tet^{R}) | This work |
| TG1recA | TG1recA56 SrlC300::Tn10 (Tet^{R}) | Laboratory collection |
| TG1ΔrseA | TG1ΔrseA::Δfrt | This work |
| TG1gfp | TG1λatt::gfp-bla, (Amp^{R}) | A gift of A. Roux |
| TG1gfpΔcpxP | TG1ΔcpxP λatt::gfp-bla (Amp^{R}) | This work |
| TG1gfpΔcpxR | TG1ΔcpxR λatt::gfp-bla (Amp^{R}) | This work |
| TG1gfpΔyccA | TG1ΔyccA λatt::gfp-bla (Km^{R}, Amp^{R}) | This work |
| TG1gfpΔycfJ* | TG1ΔycfJ λatt::gfp-bla (Km^{R}, Amp^{R}) | This work |
| Plasmids | | |
| pKOBEG | pSC101 ts (replicates at 30° C.), araC | (Chaveroche et al., |
| | arabinose-inducible λ redγβα operon, (Cm^{R}) | 2000) |
| pCP20 | ts (replicates at 30° C.) plasmid bearing the flp | (Cherepanov and |
| | recombinase gene, (Cm^{R} and Amp^{R}) | Wackernagel, 1995) |

*Additional individual mutants in the following genes: cutC, cyoC, dinI, eco, fadB, fdhF, gadA, lctR, malM-G, mdh, nifS, nifU, nlpE, pspA-E, rbsB, rpoE, rseB, sixA, sodC, spy, sucA, sulA, tatE, ybeD, ybjF, yccA, yceP, ycfJ, ycfL, ycfR, ydcI, yebE, yfcX, yggN, yghO, ygiB, yhhY, yiaH, yjbO, yneA, yoaB, yqcC, yqeC, were named TG1Δ□gene.name]::aphA, (Km^{R}).

TABLE 3
Genes over-expressed in E. coli TG1 biofilm versus exponential growth phase.

| Genes | | Rank | Bio/Exp | Function - description |
|---|---|---|---|---|
| a | b | c | d | e |

INFORMATION STORAGE AND PROCESSING
J: Translation. ribosomal structure and metabolism

| | | | | |
|---|---|---|---|---|
| lysU | b4129 | 146 | 1.45 | Lysine tRNA synthetase |
| miaA | b4171 | 79 | 1.84 | Delta(2)-isopentenylpyrophosphate tRNA-adenosine transferase |
| rluC | b1086 | 166 | 1.35 | Ribosomal large subunit pseudouridine synthase C |
| rne | b1084 | 58 | 2.06 | RNase E |
| rplY | b2185 | 52 | 2.23 | 50S ribosomal subunit protein L25 |

K: Transcription

| | | | | |
|---|---|---|---|---|
| crl | b0240 | 81 | 1.83 | Transcriptional regulator of genes for curli |
| cspD | b0880 | 135 | 1.50 | Cold shock protein |
| dniR | b0211 | 199 | 1.24 | Transcriptional regulator for nitrite reductase |
| fruR | b0080 | 133 | 1.50 | Transcriptional repressor of fru operon and others |
| idnR | b4264 | 227 | 1.18 | L-idonate transcriptional regulator |
| lacI | b0345 | 170 | 1.34 | Transcriptional repressor of the lac operon |
| → lctR | b3604 | 14 | 4.76 | Regulatory protein for L-Lactate dehydrogenase genes |
| nac | b1988 | 105 | 1.66 | Nitrogen assimilation control protein |
| rnk | b0610 | 126 | 1.53 | Regulator of nucleoside diphosphate kinase |
| rpoS | b2741 | 88 | 1.78 | RNA polymerase sigma S factor |
| ttk | b3641 | 134 | 1.50 | Putative transcriptional regulator |

L: DNA replication. recombination and repair

| | | | | |
|---|---|---|---|---|
| b0299 | b0299 | 245 | 1.13 | IS3 putative transposase |
| dinG | b0799 | 114 | 1.60 | ATP-dependent helicase. SOS |
| dinI | b1061 | 61 | 2.02 | Inhibits RecA-mediated self-cleavage. SOS |
| dinP | b0231 | 82 | 1.81 | Putative tRNA synthetase. SOS |
| exo | b2798 | 78 | 1.84 | 5′-3′ exonuclease. excision repair |
| intA | b2622 | 177 | 1.31 | Prophage CP4-57 integrase |
| → recA | b2699 | 51 | 2.30 | DNA strand exchange and renaturation. SOS |
| recN | b2616 | 237 | 1.16 | Recombination and DNA repair. SOS |
| sbmC | b2009 | 94 | 1.74 | SbmC protein. SOS |
| xthA | b1749 | 207 | 1.23 | Exonuclease III |

METABOLISM
C: Energy production and conversion

| | | | | |
|---|---|---|---|---|
| aceA | b4015 | 195 | 1.25 | Isocitrate lyase |
| aceB | b4014 | 120 | 1.56 | Malate synthase A |
| aldA | b1415 | 67 | 1.93 | Aldehyde dehydrogenase. NAD-linked |
| atpA | b3734 | 186 | 1.28 | Membrane-bound ATP synthase alpha-subunit |
| cyoA | b0432 | 89 | 1.78 | Cytochrome o ubiquinol oxidase subunit II |
| cyoC | b0430 | 26 | 3.59 | Cytochrome o ubiquinol oxidase subunit III |
| cyoD | b0429 | 3 | 7.41 | Cytochrome o ubiquinol oxidase subunit IV |
| dctA | b3528 | 74 | 1.89 | Uptake of C4-dicarboxylic acids |
| fdhF | b4079 | 23 | 3.85 | Subunit of formate dehydrogenase H. |
| fdoG | b3894 | 69 | 1.92 | Formate dehydrogenase-O major subunit |
| glpD | b3426 | 178 | 1.31 | Sn-glycerol-3-phosphate dehydrogenase |
| glpK | b3926 | 92 | 1.75 | Glycerol kinase |
| glpQ | b2239 | 46 | 2.50 | Glycerol-3-phosphate diesterase |
| → mdh | b3236 | 53 | 2.19 | Malate dehydrogenase |
| nifU | b2529 | 27 | 3.41 | Formation/repair of[Fe—S] clusters present in iron-sulfur proteins |
| pckA | b3403 | 112 | 1.62 | Phosphoenolpyruvate carboxykinase |
| sdhB | b0724 | 173 | 1.32 | Succinate dehydrogenase. Iron sulfur protein |
| sdhD | b0722 | 131 | 1.52 | Succinate dehydrogenase. Hydrophobic subunit |
| sixA | b2340 | 41 | 2.74 | Phosphohistidine phosphatase affecting phosphorelay of ArcB |
| sucA | b0726 | 7 | 6.54 | 2-oxoglutarate dehydrogenase (decarboxylase) |
| sucB | b0727 | 83 | 1.81 | 2-oxoglutarate dehydrogenase (dihydrolipoyltranssuccinate) |
| sucD | b0729 | 43 | 2.69 | Succinyl-CoA synthetase. Alpha subunit |
| xdhD | b2881 | 115 | 1.59 | Putative dehydrogenase |

G: Carbohydrate transport and metabolism

| | | | | |
|---|---|---|---|---|
| agp | b1002 | 85 | 1.80 | Periplasmic glucose-1-phosphatase |
| gcd | b0124 | 197 | 1.25 | Glucose dehydrogenase |
| glgS | b3049 | 168 | 1.35 | Glycogen biosynthesis. rpoS dependent |
| glpX | b3925 | 140 | 1.47 | Unknown function in glycerol metabolism |
| lamB | b4036 | 36 | 2.94 | Maltose high-affinity receptor |
| malE | b4034 | 91 | 1.76 | Periplasmic maltose-binding protein |
| malF | b4033 | 87 | 1.79 | Part of maltose permease |
| malS | b3571 | 164 | 1.36 | Alpha-amylase |
| mglA | b2149 | 215 | 1.21 | ATP-binding galactose-binding transport protein |
| mglB | b2150 | 66 | 1.93 | Galactose-binding transport protein |
| mrsA | b3176 | 111 | 1.62 | Similar to phosphoglucomutases and phosphomannomutases |
| pgm | b0688 | 149 | 1.45 | Phosphoglucomutase |
| → rbsB | b3751 | 48 | 2.41 | D-ribose periplasmic binding protein, chemotaxis |
| rbsC | b3750 | 159 | 1.41 | D-ribose high-affinity transport system |
| rbsD | b3748 | 154 | 1.42 | D-ribose high-affinity transport system |
| sfsA | b0146 | 64 | 1.97 | Regulatory for maltose metabolism |

E: Amino acid transport and metabolism

| | | | | |
|---|---|---|---|---|
| ansB | b2957 | 65 | 1.96 | Periplasmic L-asparaginase II |
| argC | b3958 | 201 | 1.24 | N-acetyl-gamma-glutamylphosphate reductase |
| argR | b3237 | 137 | 1.49 | Repressor of arg regulon |
| gadA | b3517 | 30 | 3.15 | Glutamate decarboxylase isozyme |
| idnD | b4267 | 175 | 1.31 | L-idonate dehydrogenase |
| leuD | b0071 | 106 | 1.66 | Isopropylmalate isomerase subunit |
| metH | b4019 | 72 | 1.90 | Repressor of metE and metF |
| nifS | b2530 | 62 | 1.98 | Cysteine desulfurase |
| putP | b1015 | 183 | 1.29 | Major sodium/proline symporter |

F: Nucleotide transport and metabolism: none
H: Coenzyme metabolism

| | | | | |
|---|---|---|---|---|
| metK | b2942 | 110 | 1.63 | Methionine adenosyltransferase |
| pnuC | b0751 | 169 | 1.34 | Required for NMN transport |
| ubiE | b3833 | 220 | 1.20 | Ubiquinone/menaquinone biosynthesis methyltransferase |

I: Lipidmetabolism:

| | | | | |
|---|---|---|---|---|
| fabA | b0954 | 127 | 1.53 | Trans-2-decenoyl-ACP isomerase |
| fadB | b3846 | 21 | 4.18 | Fatty acid oxidation complex. 4-enzyme protein |
| fadE | b0221 | 84 | 1.80 | Acyl-coenzyme A dehydrogenase |
| fadL | b2344 | 181 | 1.29 | Transport of long-chain fatty acids |
| pgpA | b0418 | 102 | 1.67 | Phosphatidylglycerophosphatase |
| pssA | b2585 | 239 | 1.15 | Phosphatidylserine synthase. Phospholipid synthesis |
| uppS | b0174 | 156 | 1.41 | Undecaprenyl pyrophosphate synthetase (peptidoglycan) |

Q: Secondary metabolites biosynthesis. transport and metabolism

| | | | | |
|---|---|---|---|---|
| idnO | b4266 | 148 | 1.45 | 5-keto-D-gluconate 5-reductase |
| ucpA | b2426 | 50 | 2.32 | Short-chain dehydrogenases/reductases (SDR) family |

CELLULAR PROCESSES
D: Cell division and chromosomal partitioning

| | | | | |
|---|---|---|---|---|
| fisL | b0083 | 17 | 4.34 | Cell division and growth |
| sulA | b0958 | 33 | 3.07 | Inhibits cell division and ftsZ ring formation. SOS |

O: Post-translational modification. protein turnover, chaperones

| | | | | |
|---|---|---|---|---|
| dnaJ | b0015 | 163 | 1.36 | Chaperone with DnaK. Heat shock protein |
| dnaK | b0014 | 107 | 1.64 | Chaperone Hsp70. Heat shock proteins |
| eco | b2209 | 9 | 6.17 | Ecotin. Periplasmic serine protease inhibitor |
| fkpA | b3347 | 93 | 1.74 | FKBP-type peptidyl-prolyl cis-trans isomerase (rotamase) |
| glnE | b3053 | 100 | 1.69 | Adenylylating enzyme for glutamine synthetase |
| htpG | b0473 | 218 | 1.20 | Chaperone Hsp90. Heat shock protein |
| htpX | b1829 | 76 | 1.88 | Heat shock protein. Integral membrane protein |
| → msrA | b4219 | 22 | 3.87 | Peptide methionine sulfoxide reductase |

M + N: Cell envelope biogenesis and secretion

| | | | | |
|---|---|---|---|---|
| amiB | b4169 | 243 | 1.13 | N-acetylmuramoyl-1-alanine amidase II. Murein hydrolase |
| ddg | b2378 | 124 | 1.54 | Putative heat shock protein |
| fhiA | b0229 | 71 | 1.91 | Flagellar biosynthesis |
| → fimA | b4314 | 28 | 3.29 | Major type 1 subunit fimbrin (pilin) |
| fimI | b4315 | 49 | 2.33 | Fimbrial protein |
| htrL | b3618 | 214 | 1.21 | Involved in lipopolysaccharide biosynthesis |
| lepB | b2568 | 121 | 1.56 | Leader peptidase (signal peptidase 1) |
| mraW | b0082 | 238 | 1.15 | Putative apolipoprotein |
| nlpB | b2477 | 191 | 1.26 | Lipoprotein-34 |
| nlpC | b1708 | 182 | 1.29 | Lipoprotein |
| ompC | b2215 | 129 | 1.52 | Outer membrane protein 1b |
| ompG | b1319 | 162 | 1.37 | Outer membrane protein G |
| pspA | b1304 | 2 | 8.42 | Phage shock protein. Inner membrane protein |
| pspB | b1305 | 59 | 2.04 | Phage shock protein |
| pspC | b1306 | 12 | 5.58 | Phage shock protein. Activates phage shock-protein expression |
| pspD | b1307 | 11 | 5.61 | Phage shock protein |
| pspE | b1308 | 47 | 2.47 | Phage shock protein |
| → pspF | b1303 | 211 | 1.22 | psp operon transcriptional activator |
| → tatE | b0627 | 57 | 2.12 | Membrane translocation of folded periplasmic proteins |

P: Inorganic ion transport and metabolism

| | | | | |
|---|---|---|---|---|
| chaA | b1216 | 234 | 1.16 | Sodium-calcium/proton antiporter |
| chaC | b1218 | 70 | 1.91 | Accessory and regulatory protein for chaA |
| cutC | b1874 | 24 | 3.74 | Copper homeostasis protein |
| cysP | b2425 | 74 | 1.89 | Thiosulfate binding protein |
| cysU | b2424 | 136 | 1.49 | Thiosulfate transport system permease |
| fur | b0683 | 188 | 1.27 | Ferric iron uptake negative regulator |
| modA | b0763 | 99 | 1.70 | Molybdate-binding periplasmic protein. Permease |
| modB | b0764 | 223 | 1.19 | Molybdate transport permease protein |
| modC | b0765 | 216 | 1.20 | ATP-binding component of molybdate transport |
| modE | b0761 | 139 | 1.47 | Molybdate uptake regulatory protein |
| sodC | b1646 | 32 | 3.10 | Superoxide dismutase precursor (Cu—Zn) |
| trkH | b3849 | 226 | 1.19 | Potassium uptake |

T: Signal transduction mechanism:

| | | | | |
|---|---|---|---|---|
| → cpxP | b3914 | 1 | 22.9 | Suppresses toxic envelope protein effects. CpxA/R activated |
| rpoE | b2573 | 63 | 1.98 | Extra-cytoplasmic Sigma-E factor |
| rseA | b2572 | 55 | 2.15 | Negative regulatory protein of sigma-E factor |
| rseB | b2571 | 29 | 3.20 | Negative regulatory protein of sigma-E factor |
| → spy | b1743 | 31 | 3.13 | Periplasmic protein related to spheroblast formation |

NOT CHARACTERIZED
R: Function unknown: General prediction only

| | | | | |
|---|---|---|---|---|
| → ycfJ | b1110 | 5 | 6.95 | Similarity to Rickettsia 17 kda surface antigen |
| → ycfR | b1112 | 8 | 6.4 | Exported/Outer membrane protein? |
| yoaB | b1809 | 10 | 6.03 | Putative translation initiation inhibitor |
| yebE | b1846 | 13 | 5.47 | Similarity to an Y. enterocolitica protein |
| → yqcC | b2792 | 15 | 4.45 | Similarity to E. carotovora orf1 exoenzyme |
| yhhY | b3441 | 16 | 4.35 | Putative acetyltransferase |
| → yggN | b2958 | 21 | 4.1 | Activated by RpoE |
| → yneA | b1516 | 25 | 3.6 | Putative periplasmic binding protein |
| ybeD | b0631 | 35 | 2.97 | Homology to one histine kinase sensor domain of M. grisea |
| ydcI | b1422 | 37 | 2.83 | Putative transcriptional regulator LysR-type |
| yddL | b1472 | 38 | 2.82 | Putative outer membrane porin protein |
| → yccA | b0970 | 39 | 2.76 | Putative carrier/transport membrane protein. Degraded by FtsH |
| → yfcX | b2341 | 40 | 2.75 | Putative fatty oxidation complex alpha subunit |
| yjbO | b4050 | 44 | 2.58 | Similarity to a putative exported Y. pestis protein |
| yrdD | b3283 | 45 | 2.54 | Putative DNA topoisomerase |
| ybjF | b0859 | 56 | 2.14 | Putative 23S rRNA (uracil-5-)-methyltransferase |
| yihN | b3874 | 60 | 2.03 | Putative resistance protein (transport) |
| ycfT | b1115 | 68 | 1.93 | Integral membrane protein |
| yeeF | b2014 | 77 | 1.84 | Putative amino acid/amine transport protein |
| yfiE | b2577 | 86 | 1.79 | Putative transcriptional regulator LysR-type |
| yeeD | b2012 | 90 | 1.77 | Belongs to the UPF0033 family |
| yliH | b0836 | 95 | 1.73 | Putative receptor |
| yfcM | b2326 | 96 | 1.73 | Putative transporting ATPase |
| ybiX | b0804 | 97 | 1.73 | Putative enzyme |
| yfhF/nifA | b2528 | 101 | 1.68 | Putative regulator |
| ygfQ | b2884 | 113 | 1.62 | Belongs to YicO/YieG/YjcD family |
| ybhR | b0792 | 118 | 1.58 | Simlarity to E. coli YbhS, YhhJ and YhiG. IM protein |
| ybdH | b0599 | 130 | 1.52 | Putative oxidoreductase |
| yihR | b3879 | 132 | 1.51 | Putative aldose-1-epimerase |
| ydcT | b1441 | 138 | 1.47 | Putative ATP-binding component of a transport system |
| ygiS | b3020 | 143 | 1.45 | Putative transport periplasmic protein |
| ybaZ | b0454 | 150 | 1.44 | Similarity to Cysteine methyltransferase |
| ydaM | b1341 | 155 | 1.41 | Contains 1 GGDEF Duf1 domain |
| tfaR | b1373 | 157 | 1.41 | Phage lambda tail fiber gene homolog in prophage Rac |
| yceL | b1065 | 161 | 1.38 | Belongs to the major fator family. Integral Membrane Protein |
| yheT | b3353 | 167 | 1.35 | Belongs to the UPF0017 family |
| yjdC | b4135 | 172 | 1.33 | Similarity to S. glaudescens TcmR |
| ybiW | b0823 | 174 | 1.32 | Putative formate acetyltransferase |
| ybiF | b0813 | 176 | 1.31 | Putative transmembrane subunit |
| ynaI | b1330 | 180 | 1.3 | Belongs to the UPF0003 family. Integral membrane protein |
| yceE | b1053 | 185 | 1.28 | Putative transport protein |
| yhdP | b1657 | 196 | 1.25 | Putative transport protein |
| ygjE | b3063 | 213 | 1.21 | Putative tartrate carrier |
| csiE | b2535 | 217 | 1.2 | Stationary phase inducible protein |
| yfdE | b2371 | 219 | 1.2 | Putative enzyme |
| yeeE | b2013 | 221 | 1.19 | Putative transport system permease protein |
| yegQ | b2081 | 228 | 1.18 | Putative peptidase (family U32) |
| glcA | b2975 | 229 | 1.17 | Putative permease |
| yfdW | b2374 | 232 | 1.17 | Putative enzyme |
| yfeT | b2427 | 233 | 1.17 | Belongs to the Sis family, RpiR subfamily |
| ygjK | b3080 | 236 | 1.16 | Putative isomerase |
| ydeW | b1512 | 242 | 1.13 | Putative transcriptional regulator. SorC family |

S: Function unknown

| | | | | |
|---|---|---|---|---|
| b1228 | b1228 | 4 | 7.04 | Unknown |
| ycfL | b1104 | 6 | 6.70 | Unknown |
| → yghO | b2981 | 18 | 4.31 | Unknown |
| yiaH | b3561 | 19 | 4.18 | Unknown. Integral membrane protein |
| → yceP | b1060 | 34 | 3.06 | Unknown |
| yqeC | b2876 | 42 | 2.70 | Unknown |
| □ygiB | b3037 | 54 | 2.15 | Unknown |
| ycfT | b1115 | 68 | 1.93 | Unknown. Integral membrane protein |
| yhjJ | b3527 | 73 | 1.90 | Unknown |
| yceB | b1063 | 80 | 1.84 | Unknown |
| ybiX | b0804 | 97 | 1.73 | Unknown |
| ygiQ | b3015 | 98 | 1.71 | Unknown |
| yagV | b0289 | 103 | 1.67 | Unknown |
| yoeA | b1995 | 104 | 1.66 | Unknown |
| ybhQ | b0791 | 108 | 1.64 | Unknown |
| ybcI | b0527 | 109 | 1.63 | Unknown |
| ybbF | b0524 | 116 | 1.59 | Unknown |
| ybgI | b0710 | 117 | 1.58 | Unknown |
| yncH | b1455 | 119 | 1.58 | Unknown |
| yfbM | b0681 | 122 | 1.56 | Unknown |
| yjiM | b4335 | 123 | 1.54 | Unknown |
| yjfO | b4189 | 125 | 1.54 | Unknown |
| ychN | b1219 | 128 | 1.53 | Unknown |
| ynaC | b1373 | 141 | 1.47 | Unknown |
| ymfE | b1138 | 142 | 1.46 | Unknown |
| yfcN | b2331 | 144 | 1.45 | Unknown |
| yrbC | b3192 | 145 | 1.45 | Unknown |
| yfdQ | b2360 | 147 | 1.45 | Unknown |
| yfeY | b2432 | 151 | 1.44 | Unknown |
| ygiM | b3055 | 152 | 1.43 | Unknown |
| yhgA | b3411 | 153 | 1.43 | Unknown |
| yhjQ | b3534 | 158 | 1.41 | Unknown |
| yfcF | b2301 | 160 | 1.39 | Unknown |
| yfcI | b2305 | 165 | 1.35 | Unknown |
| yjiD | b4326 | 171 | 1.34 | Unknown |
| yfbP | b2275 | 179 | 1.30 | Unknown |
| yphB | b2544 | 184 | 1.28 | Unknown |
| yfbN | b2273 | 187 | 1.28 | Unknown |
| ylbH | b0499 | 189 | 1.27 | Unknown |
| ybhM | b0787 | 190 | 1.26 | Unknown. Integral membrane protein |
| yrbL | b3207 | 192 | 1.26 | Unknown |
| yjfY | b4199 | 193 | 1.25 | Unknown |
| ynfA | b1582 | 194 | 1.25 | Unknown |
| yajI | b0412 | 198 | 1.25 | Unknown |
| yedI | b1958 | 200 | 1.24 | Unknown |
| yafZ | b0252 | 202 | 1.24 | Unknown |
| yjjU | b4377 | 203 | 1.24 | Unknown |
| yfhH | b2561 | 204 | 1.24 | Unknown |
| yafN | b0232 | 205 | 1.23 | Unknown |
| yrbE | b3194 | 206 | 1.23 | Unknown |
| yfgC | b2494 | 208 | 1.22 | Unknown |
| yfjQ | b2633 | 209 | 1.22 | Unknown |
| ycaK | b0901 | 210 | 1.22 | Unknown |
| yfeS | b2420 | 212 | 1.22 | Unknown |
| b4250 | b4250 | 222 | 1.19 | Unknown |
| ybgA | b0707 | 224 | 1.19 | Unknown |
| yeeA | b2008 | 225 | 1.19 | Unknown |
| ypfI | b2474 | 230 | 1.17 | Unknown |
| b2394 | b2394 | 231 | 1.17 | IS186 hypothetical protein |
| yegK | b2072 | 235 | 1.16 | Unknown |
| ybcJ | b0528 | 240 | 1.14 | Unknown |
| yhiN | b3492 | 241 | 1.14 | Unknown |
| ypfG | b2466 | 244 | 1.13 | Unknown |
| ydiY | b1722 | 246 | 1.13 | Unknown |
| yjjJ | b4385 | 247 | 1.12 | Unknown |
| ycaP | b0906 | 248 | 1.11 | Unknown |
| yfgJ | b2510 | 249 | 1.10 | Unknown |

The genes found to be over-expressed at a significant level (P-value ≦0.05) are indicated. They have been classified according to the COGs functional categories annotation system.
a: Gene names according to E. coli Colibri database.
b: Gene names according to Blattner nomenclature.
c: Ranking position 1 = the most over-expressed gene in E. coli biofilm.
d: Ratio of gene expression in E. coli biofilm versus gene expression in planktonic cultures.
e: Function description according to E. coli Colibri database.
Arrow: mutants affected for biofilm formation.

TABLE 4
Genes under-expressed in E. coli TG1 biofilm versus exponential growth phase.

| Genes | | Rank | Bio/Exp | Function - description |
|---|---|---|---|---|
| a | b | c | d | e |

INFORMATION STORAGE AND PROCESSING
J: Translation, ribosomal structure and metabolism

| | | | | |
|---|---|---|---|---|
| def | b3287 | 62 | 0.67 | Peptide deformylase |
| frr | b0172 | 81 | 0.70 | Ribosome releasing factor |
| prfA | b1211 | 166 | 0.84 | Peptide chain release factor RF-1 |
| rbfA | b3167 | 40 | 0.61 | Ribosome-binding factor A |
| rbn | b3886 | 176 | 0.87 | tRNA processing exoribonuclease BN |
| rimJ | b1066 | 88 | 0.72 | Acetylation of 30S ribosomal subunit protein S5 |
| rpmG | b3636 | 79 | 0.69 | 50S ribosomal subunit protein L33 |
| rpsV | b1480 | 161 | 0.83 | 30S ribosomal subunit protein S22 |
| serS | b0893 | 96 | 0.73 | Serine tRNA synthetase |
| thrS | b1719 | 63 | 0.67 | Threonine tRNA synthetase |

K: Transcription

| | | | | |
|---|---|---|---|---|
| gcvR | b2479 | 69 | 0.67 | Transcriptional regulation of gcv operon |
| malT | b3418 | 119 | 0.77 | Positive regulator of mal regulon |
| osmE | b1739 | 66 | 0.67 | Osmotically inducible lipoprotein E |
| oxyR | b3961 | 108 | 0.75 | Activator of hydrogen peroxide-inducible genes |
| xylR | b3569 | 170 | 0.86 | Putative regulator of xyl operon |

L: DNA replication, recombination and repair

| | | | | |
|---|---|---|---|---|
| dnaG | b3066 | 187 | 0.92 | DNA primase |
| holA | b0640 | 134 | 0.79 | DNA polymerase III delta subunit |
| hupB | b0440 | 41 | 0.61 | DNA-binding protein HU-beta |
| intE | b1140 | 164 | 0.84 | Prophage e14 integrase |
| nudG | b1759 | 174 | 0.87 | CTP pyrophosphohydrolase |
| uvrD | b3813 | 183 | 0.91 | DNA-dependent ATPase I and helicase II |
| xerC | b3811 | 109 | 0.76 | Site-specific recombinase |

METABOLISM
C: Energy production and conversion

| | | | | |
|---|---|---|---|---|
| adhE | b1241 | 1 | 0.23 | Iron-dependent alcohol dehydrogenase |
| aldB | b3588 | 180 | 0.89 | Aldehyde dehydrogenase |
| cydA | b0733 | 93 | 0.73 | Cytochrome d terminal oxidase. Polypeptide subunit I |
| cydB | b0734 | 127 | 0.78 | Cytochrome d terminal oxidase Polypeptide subunit II |
| dcuC | b0621 | 106 | 0.75 | Transport of dicarboxylates |
| fumB | b4122 | 145 | 0.81 | Fumarase B |
| icdA | b1136 | 136 | 0.80 | Isocitrate dehydrogenase |
| pflB | b0903 | 29 | 0.56 | Formate acetyltransferase 1 |
| pta | b2297 | 120 | 0.77 | Phosphotransacetylase |

G: Carbohydrate transport and metabolism

| | | | | |
|---|---|---|---|---|
| bglX | b2132 | 159 | 0.83 | Beta-D-glucoside glucohydrolase |
| cmr | b0842 | 158 | 0.83 | Proton motive force efflux pump |
| cpsG | b2048 | 135 | 0.80 | Phosphomannomutase |
| crr | b2417 | 70 | 0.68 | Glucose-specific IIA component |
| eno | b2779 | 27 | 0.55 | Enolase. Glycolysis |
| fba | b2925 | 48 | 0.63 | Fructose-bisphosphate aldolase. Glycolysis |
| fbp | b4232 | 103 | 0.75 | Fructose-bisphosphatase |
| fruA | b2167 | 61 | 0.66 | Fructose-specific transport protein |
| fruB | b2169 | 71 | 0.68 | Fructose-specific IIA/fpr component |
| fruK | b2168 | 33 | 0.57 | Fructose-1-phosphate kinase |
| gapA | b1779 | 4 | 0.32 | Glyceraldehyde-3-phosphate dehydrogenase A. |
| gpmA | b0755 | 36 | 0.60 | Phosphoglyceromutase 1. Glycolysis |
| manY | b1818 | 18 | 0.47 | PTS enzyme IIC. Mannose-specific |
| nagZ | b1107 | 94 | 0.73 | Beta-hexosaminidase. Cell wall synthesis |
| pfkA | b3916 | 56 | 0.66 | 6-phosphofructokinase I. Glycolysis |
| pgk | b2926 | 53 | 0.65 | Phosphoglycerate kinase. Glycolysis |
| ptsI | b2416 | 47 | 0.62 | PEP-protein phosphotransferase system enzyme I |
| sgaH | b4196 | 90 | 0.72 | Hexulose-6-phosphate synthase |
| sgaU | b4197 | 146 | 0.82 | Hexulose-6-phosphate isomerase |
| shiA | b1981 | 144 | 0.81 | Putative shikimate transport protein |
| torT | b0994 | 38 | 0.60 | Part of regulation of tor operon. |
| tpiA | b3919 | 45 | 0.62 | Triosephosphate isomerase. Glycolysis |

E: Amino acid transport and metabolism

| | | | | |
|---|---|---|---|---|
| arcC | b0521 | 80 | 0.69 | Putative carbamate kinase. Arginine degradation |
| argF | b0273 | 102 | 0.74 | Ornithine carbamoyltransferase 2 |
| aroG | b0754 | 11 | 0.43 | DAHP synthetase. Aromatic amino acids biosynthesis |
| aroH | b1704 | 143 | 0.81 | DAHP synthetase. Aromatic amino acids biosynthesis |
| asnB | b0674 | 148 | 0.82 | Asparagine synthetase B |
| edd | b1851 | 162 | 0.84 | 6-phosphogluconate dehydratase |
| ggt | b3447 | 186 | 0.92 | Gamma-glutamyltranspeptidase |
| glnA | b3870 | 58 | 0.66 | Glutamine synthetase |
| glnB | b2553 | 20 | 0.49 | Regulatory protein P-II for glutamine synthetase |
| hisB | b2022 | 25 | 0.53 | Imidazole glycerolphosphate dehydratase |
| hisC | b2021 | 17 | 0.47 | Histidinol-phosphate aminotransferase |
| hisG | b2019 | 165 | 0.84 | ATP phosphoribosyltransferase |
| hisI | b2026 | 44 | 0.62 | Phosphoribosyl-ATP pyrophosphatase |
| ilvL | b3766 | 14 | 0.45 | ilvGEDA operon leader peptide |
| oppA | b1243 | 42 | 0.61 | Oligopeptide transport. Periplasmic binding protein |
| oppB | b1244 | 74 | 0.68 | Oligopeptide transport. Permease protein |
| oppC | b1245 | 129 | 0.78 | Oligopeptide transport. Permease protein |
| oppD | b1246 | 65 | 0.67 | ATP-binding protein of oligopeptide transport system |
| oppF | b1247 | 172 | 0.86 | ATP-binding protein of oligopeptide transport system |
| pepQ | b3847 | 101 | 0.74 | Proline dipeptidase |
| trpA | b1260 | 72 | 0.68 | Tryptophan synthase. alpha protein |
| trpB | b1261 | 39 | 0.60 | Tryptophan synthase. beta protein |

F: Nucleotide transport and metabolism

| | | | | |
|---|---|---|---|---|
| cyaA | b3806 | 43 | 0.61 | Adenylate cyclase |
| hpt | b0125 | 59 | 0.66 | Purine salvage |
| tdk | b1238 | 113 | 0.77 | Thymidine kinase |

H: Coenzyme metabolism

| | | | | |
|---|---|---|---|---|
| bioH | b3412 | 118 | 0.77 | Biotin biosynthesis |
| dxs | b0420 | 150 | 0.82 | 1-deoxyxylulose-5-phosphate synthase. Flavoprotein |
| folC | b2315 | 179 | 0.89 | Dihydrofolate synthetase |
| mobA | b3857 | 49 | 0.63 | Molybdopterin |
| tbpA | b0068 | 76 | 0.69 | Thiamin-binding periplasmic protein |

I: Lipid metabolism: none
Q: Secondary metabolites biosynthesis, transport and metabolism

| | | | | |
|---|---|---|---|---|
| pmbA | b4235 | 149 | 0.82 | Maturation of antibiotic MccB17 |

CELLULAR PROCESSES
D: Cell division and chromosomal partitioning

| | | | | |
|---|---|---|---|---|
| zipA | b2412 | 126 | 0.78 | Cell division protein involved in FtsZ ring |

O: Post-translational modification, protein turnover, chaperones

| | | | | |
|---|---|---|---|---|
| clpA | b0882 | 133 | 0.79 | ATP-binding component of serine protease |
| fkpB | b0028 | 50 | 0.64 | Peptidyl-prolyl cis-trans isomerase (a rotamase) |
| ppiB | b0525 | 52 | 0.64 | Peptidyl-prolyl cis-trans isomerase B (rotamase B) |

M + N: Cell envelope biogenesis and secretion

| | | | | |
|---|---|---|---|---|
| cpsB | b2049 | 91 | 0.72 | Colanic acid biosynthesis |
| dacA | b0632 | 34 | 0.58 | D-alanyl-D-alanine carboxypeptidase |
| exbB | b3006 | 16 | 0.46 | Uptake of enterochelin |
| exbD | b3005 | 15 | 0.46 | Uptake of enterochelin |
| lpp | b1677 | 24 | 0.53 | Murein lipoprotein |
| lpxD | b0179 | 84 | 0.71 | Third step of endotoxin (lipidA) synthesis |
| pbpG | b2134 | 128 | 0.78 | Penicillin-binding protein 7 |
| sohB | b1272 | 60 | 0.66 | Putative protease |
| yfbE | b2253 | 64 | 0.67 | Putative enzyme |

P: Inorganic ion transport and metabolism

| | | | | |
|---|---|---|---|---|
| bfd | b3337 | 10 | 0.41 | Iron storage and mobility [2Fe—2S] |
| feoA | b3408 | 12 | 0.43 | Ferrous iron transport protein A |
| feoB | b3409 | 57 | 0.66 | ferrous iron transport protein B |
| fhuF | b4367 | 31 | 0.57 | Ferric hydroxamate transport |
| focA | b0904 | 2 | 0.30 | Formate transporter |
| hcaAl | b2538 | 86 | 0.72 | Large subunit of phenylpropionate dioxygenase |

T: Signal transduction mechanism: none
NOT CHARACTERIZED
R: Function unknown: General prediction only

| | | | | |
|---|---|---|---|---|
| yncE | b1452 | 5 | 0.34 | Putative receptor |
| yhiX | b3516 | 8 | 0.39 | Putative AraC-type regulatory protein |
| yfiD | b2579 | 13 | 0.44 | Putative formate acetyltransferase |
| yodB | b1974 | 21 | 0.51 | Putative cytochrome |
| ynfK | b1593 | 22 | 0.52 | Putative dethiobiotin synthetase |
| ycgT | b1200 | 26 | 0.53 | Putative dihydroxyacetone kinase |
| yebL | b1857 | 35 | 0.59 | Putative high-affinity zinc uptake system protein |
| yeeX | b2007 | 37 | 0.60 | Putative alpha helix protein |
| ykgM | b0296 | 51 | 0.64 | Putative ribosomal protein |
| ybaO | b0447 | 54 | 0.65 | Putative Lrp-like transcriptional regulator |
| etp | b0982 | 55 | 0.66 | Putative protein-tyrosine-phosphatase |
| ygjH | b3074 | 67 | 0.67 | Putative tRNA synthetase |
| yqhC | b3010 | 68 | 0.67 | Putative AraC-type regulatory protein |
| yhcJ | b3223 | 73 | 0.68 | Putative enzyme |
| yeiA | b2147 | 77 | 0.69 | Putative oxidoreductase |
| ybgS | b0753 | 82 | 0.71 | Putative homeobox protein |
| yhfW | b3380 | 85 | 0.71 | Putative mutase |
| ydgF | b1600 | 98 | 0.74 | Possible chaperone |
| ybcC | b0539 | 99 | 0.74 | Putative exonuclease |
| ybjW | b0873 | 100 | 0.74 | Putative prismane |
| yjiL | b4334 | 105 | 0.75 | Putative enzyme |
| ctsA | b0598 | 112 | 0.76 | Putative carbon starvation protein |
| ydjG | b1771 | 114 | 0.77 | Hypothetical oxidoreductase |
| yeaU | b1800 | 116 | 0.77 | Putative tartrate dehydrogenase |
| ygjU | b3089 | 117 | 0.77 | Putative symporter protein |
| yejO | b2190 | 121 | 0.78 | Putative ATP-binding component of a transport system |
| yeiC | b2166 | 124 | 0.78 | Putative sugar kinase |
| ynjE | b1757 | 130 | 0.79 | Putative thiosulfate sulfur transferase |
| yjbC | b4022 | 142 | 0.81 | Putative pseudo-uridine synthase |
| yadF | b0126 | 147 | 0.82 | Putative carbonic anhydrase |
| essD | b0554 | 151 | 0.82 | Lysis protein homolog to lambdoid prophage DLP12 |
| yneI | b1525 | 152 | 0.82 | Putative aldehyde dehydrogenase |
| perM | b2493 | 156 | 0.83 | Putative permease |
| yjjP | b4364 | 157 | 0.83 | Putative structural protein |
| yhdX | b3269 | 160 | 0.83 | Putative transport system permease protein |
| yihO | b3876 | 167 | 0.85 | Putative permease |
| ycgS | b1199 | 169 | 0.85 | Putative dihydroxyacetone kinase |
| yqhH | b3014 | 173 | 0.86 | Putative lipoprotein |
| b0878 | b0878 | 175 | 0.87 | Putative membrane protein |
| ygfH | b2920 | 177 | 0.88 | Putative coenzyme A transferase |
| yegH | b2063 | 178 | 0.88 | Putative transport protein |
| ydiF | b1694 | 181 | 0.89 | Putative enzyme |
| yeeZ | b2016 | 182 | 0.90 | Putative enzyme of sugar metabolism |
| ydhM | b1649 | 185 | 0.91 | Putative transcriptional regulator |
| ydjK | b1775 | 188 | 0.93 | Putative transport protein |

S: Function unknown

| | | | | |
|---|---|---|---|---|
| b3007 | b3007 | 3 | 0.30 | Unknown |
| yfjF | b2618 | 6 | 0.35 | Unknown |
| ynaK | b1365 | 7 | 0.37 | Unknown |
| b3004 | b3004 | 9 | 0.39 | Unknown |
| yodA | b1973 | 19 | 0.48 | Unknown |
| ymfA | b1122 | 23 | 0.52 | Unknown |
| yjgD | b4255 | 28 | 0.56 | Unknown |
| yeaQ | b1795 | 30 | 0.57 | Unknown |
| yaiI | b0387 | 32 | 0.57 | Unknown |
| yceD | b1088 | 46 | 0.62 | Unknown |
| b0100 | b0100 | 75 | 0.69 | Unknown |
| ydcN | b1434 | 78 | 0.69 | Unknown |
| ygiH | b3059 | 83 | 0.71 | Unknown |
| ytfI | b4215 | 87 | 0.72 | Unknown |
| ymfO | b1151 | 89 | 0.72 | Unknown |
| ytfH | b4212 | 92 | 0.73 | Unknown |
| ynhA | b1679 | 95 | 0.73 | Unknown |
| ybaM | b0466 | 97 | 0.73 | Unknown |
| ynfB | b1583 | 104 | 0.75 | Unknown |
| ydgA | b1614 | 107 | 0.75 | Unknown |
| yggJ | b2946 | 110 | 0.76 | Unknown |
| yadS | b0157 | 111 | 0.76 | Unknown |
| yfeK | b2419 | 115 | 0.77 | Unknown |
| ycgR | b1194 | 122 | 0.78 | Unknown |
| yfdS | b2362 | 123 | 0.78 | Unknown |
| yadH | b0128 | 125 | 0.78 | Unknown |
| yhhZ | b3442 | 131 | 0.79 | Unknown |
| yhiJ | b3488 | 132 | 0.79 | Unknown |
| ycbJ | b0919 | 137 | 0.81 | Unknown |
| elaA | b2267 | 138 | 0.81 | Unknown |
| ybhN | b1788 | 139 | 0.81 | Unknown |
| ydgH | b1604 | 140 | 0.81 | Unknown |
| yfjR | b2634 | 141 | 0.81 | Unknown |
| ynfC | b1585 | 153 | 0.82 | Unknown |
| yhgG | b3410 | 154 | 0.82 | Unknown |
| ydjZ | b1752 | 155 | 0.83 | Unknown |
| ydjY | b1751 | 163 | 0.84 | Unknown |
| yhbV | b3159 | 168 | 0.85 | Unknown |
| b2791 | b2791 | 171 | 0.86 | Unknown |
| ynjB | b1754 | 184 | 0.91 | Unknown |

The genes found to be under-expressed at a significant level (P-value ≦ 0.05) are indicated. They have been classified according to the COGs functional categories annotation system.
a: Gene names according to E. coli Colibri database.
b: Gene names according to Blattner nomenclature.
c: Rank position 1 = the most repressed gene in E. coli biofilm.
d: Ratio of gene expression in E. coli biofilm versus gene expression in planktonic cultures.
e: Function description according to E. coli Colibri database.

TABLE 5
Genes over-expressed (≧2) in E. coli TG1 biofilm versus both exponential
and stationary growth phase.

| Genes | | Bio/Exp | Bio/Sta | Function - description |
|---|---|---|---|---|
| a | b | c | d | e |

INFORMATION STORAGE AND PROCESSING
J: Translation. ribosomal structure and metabolism

| | | | | |
|---|---|---|---|---|
| rne | b1084 | 2.06 | 3.57 | RNase E |

K: Transcription

| | | | | |
|---|---|---|---|---|
| → lctR | b3604 | 4.76 | 8.07 | Regulatory protein for L-Lactate dehydrogenase genes |

L: DNA replication. recombination and repair

| | | | | |
|---|---|---|---|---|
| dinI | b1061 | 2.02 | 2.92 | Inhibits RecA-mediated self-cleavage. SOS |

METABOLISM
C: Energy production and conversion

| | | | | |
|---|---|---|---|---|
| glpQ | b2239 | 2.50 | 2.15 | Glycerol-3-phosphate diesterase |
| → mdh | b3236 | 2.19 | 3.68 | Malate dehydrogenase |
| sixA | b2340 | 2.74 | 3.24 | Phosphohistidine phosphatase affecting phosphorelay of ArcB |

G: Carbohydrate transport and metabolism

| | | | | |
|---|---|---|---|---|
| lamB | b4036 | 2.94 | 3.73 | Maltose high-affinity receptor |
| → rbsB | b3751 | 2.41 | 3.83 | D-ribose periplasmic binding protein. chemotaxis |

E: Amino acid transport and metabolism

| | | | | |
|---|---|---|---|---|
| gadA | b3517 | 3.15 | 4.84 | Glutamate decarboxylase isozyme |

F: Nucleotide transport and metabolism: none
H: Coenzyme metabolism: none
I: Lipid metabolism: none
Q: Secondary metabolites biosynthesis. transport and metabolism: none
CELLULAR PROCESSES
D: Cell division and chromosomal partitioning: none
O: Post-translational modification. protein turnover. Chaperones: none

| | | | | |
|---|---|---|---|---|
| pspA | b1304 | 8.42 | 3.86 | Phage shock protein. Inner membrane protein |
| pspB | b1305 | 2.04 | 3.44 | Phage shock protein |
| pspC | b1306 | 5.58 | 2.55 | Phage shock protein. Activates phage shock-protein expression |
| pspD | b1307 | 5.61 | 2.48 | Phage shock protein |
| → tatE | b0627 | 2.12 | 5.02 | Membrane translocation of folded periplasmic proteins |

P: Inorganic ion transport and metabolism: none
T: Signal transduction mechanism:

| | | | | |
|---|---|---|---|---|
| → cpxP | b3914 | 22.9 | 13.15 | Suppresses toxic envelope protein effects. CpxA/R activated |
| rseA | b2572 | 2.15 | 2.28 | Negative regulatory protein of sigma-E factor |
| rpoE | b2573 | 1.98 | 5.86 | Extracytoplasmic sigma E factor |
| → spy | b1743 | 3.13 | 5.02 | Periplasmic protein related to spheroblast formation |

NOT CHARACTERIZED
R: Function unknown: General prediction only

| | | | | |
|---|---|---|---|---|
| yebE | b1846 | 5.47 | 3.14 | Similarity to an Y. enterocolitica protein |
| → yqcC | b2792 | 4.45 | 2.31 | Similarity to E. carotovora orfl exoenzyme |
| → yfcX | b2341 | 2.75 | 4.83 | Putative fatty oxidation complex alpha subunit |
| yjbO | b4050 | 2.58 | 5.66 | Similarity to a putative exported Y. pestis protein |

S: Function unknown

| | | | | |
|---|---|---|---|---|
| → yceP | b1060 | 3.06 | 6.46 | Unknown |
| → ygiB | b3037 | 2.15 | 2.09 | Unknown |

a: Gene names according to E. coli Colibri database.
b: Gene names according to Blattner nomenclature.
c: Ratio of gene expression in E. coli bioflim versus exponential growth phase.
d: Ratio of gene expression in E. coli bioflim versus stationary growth phase.
e: Function description according to E. coli Colibri database.
Arrow: mutants where bioflim formation were reduced compared to wt.
The genes have been classified according to the COGs functional categories annotation system used by the NCBI.

TABLE 6

Inactivation of the genes described in the study and TG1gfp strain
construction: primers used in the linear DNA, 3 step PCR
inactivation protocol.

| | | | |
|---|---|---|---|
| | | SEQ | |
| Target | | ID | |
| genes^{a} | Primers name^{b} | NO | Primers sequence |

| | | | |
|---|---|---|---|
| cpxP* | CpxP.A1.500-5 | 1 | 5′CGGCATCATTACGTCAAGCAAAAG3′ |
| | CpxP.B1.500-3 | 2 | 5′GCGCCAGCGCCGCGAGGGACTCAG3′ |
| | CpxP.B2.frtL-5 | 3 | 5′GAACTTCGGAATAGGAACTAATAGTAAACCCTGTTTTCCTTGCC3′ |
| | CpxP.A2.frtL-3 | 4 | 5′GAAGCAGCTCCAGCCTACACCATCATTTGCTCCCAAAATCTTTC3′ |
| | CpxP.ext-5 | 5 | 5′CCCGAATTCCGAAGTGCTTTTAATGTGTCG3′ |
| | CpxP.ext-3 | 6 | 5′CGCCTGGATCTGTCATCGGTG3′ |
| cpxR* | CpxR.A1.500-5 | 7 | 5′CGTGAGTTGCTACTACTCAATAG3′ |
| | CpxR.B1.500-3 | 8 | 5′GCCGGACGAATCAGATAAAG3′ |
| | CpxR.B2.frtL-5 | 9 | 5′GAACTTCGGAATAGGAACTAAGGTTTAAAACCTTGCGTGGTC3′ |
| | CpxR.A2.frtL-3 | 10 | 5′GAAGCAGCTCCAGCCTACACGAAATTACGTCATCAGACGTCGC3′ |
| | CpxR.ext-5 | 11 | 5′GATTGATTCATAAATACTCC3′ |
| | CpxR.ext:3 | 12 | 5′CAAACAGTAAGTTAATGAAATC3′ |
| cutC | CutC.A1.500-5 | 13 | 5′CACTATTGCATCAGAAGCGG3′ |
| | CutC.B1.500-3 | 14 | 5′CCTTTCTGGTTCGAAAAGTGG3′ |
| | CutC.B2.GBL-5 | 15 | 5′CTTCACGAGGCAGACCTCAGCGCCTGATTTTTACCGTTGCATCATGTCGC3′ |
| | CutC.A2.GBL-3 | 16 | 5′GATTTTGAGACACAACGTGGCTTTCATTTTTACTCCTTAATTACGCCGAC3′ |
| | CutC.ext-5 | 17 | 5′GGAATACCTTACATTGATGA3′ |
| | CutC.ext-3 | 18 | 5′CTTTAGATGCCTTTAATTTAG3′ |
| cyoC | CyoC.A1.500-5 | 19 | 5′CCATGCTGATGATTGCAGCC3′ |
| | CyoC.B1.500-3 | 20 | 5′CCGACGCCACAACCAGTGAC3′ |
| | CyoC.B2.GBL-5 | 21 | 5′CTTCACGAGGCAGACCTCAGCGCCTAATGAGTCATTCTACCGATCAC3′ |
| | CyoC.A2.GBL-3 | 22 | 5′GATTTTGAGACACAACGTGGCTTTCATTTTTCAGCCCTGCCTTAGTAATC3′ |
| | CyoC.ext-5 | 23 | 5′CAGGGATGACCTACTGGTGG3′ |
| | CyoC.ext-3 | 24 | 5′GGATTCGCGCCAAACCACAG3′ |
| dinI | DinI.A1.500-5 | 25 | 5′GTTTAACCGCAACCATATGC3′ |
| | DinI.B1.500-3 | 26 | 5′CGATTCCTGCTTCTAATATC3′ |
| | DinI.B2.GBL-5 | 27 | 5′CTTCACGAGGCAGACCTCAGCGCCTAATATGCAGTGATTTTTTTTGCC3′ |
| | DinI.A2.GBL-3 | 28 | 5′GATTTTGAGACACAACGTGGCTTTCATAATAGCCCCCTGTTGAA3′ |
| | DinI.ext-5 | 29 | 5′CCTGACTGCGCTGAAAGTCG3′ |
| | DinI.ext-3 | 30 | 5′GACGCCGATACTCGTTTACC3′ |
| ecO | EcO.A1.500-5 | 31 | 5′CGCCGCGTTGCAGAATGTTG3′ |
| | EcO.B1.500-3 | 32 | 5′CCGGATGTGGCGTATGCTGATAAGACGC3′ |
| | EcO.B2.GBL-5 | 33 | 5′CTTCACGAGGCAGACCTCAGCGCCCAACGCGGTAGTTCGCTAAACTGCCG3′ |
| | EcO.A2.GBL-3 | 34 | 5′GATTTTGAGACACAACGTGGCTTTCCATTTTTTTGCTTTCCTTC3′ |
| | EcO.ext-5 | 35 | 5′ATTTTTGAAATTAACGCTCG3′ |
| | EcO.ext-3 | 36 | 5′GTTGAAACCGCAACCCGTTC3′ |
| fadB | FadB.A1.500-5 | 37 | 5′GATCACTTCCACATCTTCAG3′ |
| | FadB.B1.500-3 | 38 | 5′GATTTCATTTTTAAATGCGG3′ |
| | FadB.B2.GBL-5 | 39 | 5′CTTCACGAGGCAGACCTCAGCGCCTAAGGAGTCACAATGGAACAGGTTG3′ |
| | FadB.A2.GBL-3 | 40 | 5′GATTTTGAGACACAACGTGGCTTTCATGTCAGTCTCCTGAATCC3′ |
| | FadB.ext-5 | 41 | 5′CTGGCCTCAATACCCAGTTG3′ |
| | FadB.ext-3 | 42 | 5′GTTTACTGGATCAAACGCCGGACGC3′ |
| fdhF | FdhF.A1.500-5 | 43 | 5′GTCTGCAAACGCTCAACGAC3′ |
| | FdhF.B1.500-3 | 44 | 5′GTCGTTCTCCAGATCTTCCG3′ |
| | FdhF.B2.GBL-5 | 45 | 5′CTTCACGAGGCAGACCTCAGCGCCTAATACCGTCCTTTCTACAG3′ |
| | FdhF.A2.GBL-3 | 46 | 5′GATTTTGAGACACAACGTGGCTTTCCATCGGTCTCGCTCCAGTTAATC3′ |
| | FdhF.ext-5 | 47 | 5′GCCGCTGTTTGACGGTGGAC3′ |
| | FdhF.ext-3 | 48 | 5′CGCCCAGTACTCGGAATAAC3′ |
| gadA | GadA.A1.500-5 | 49 | 5′CCTTTGAACCGTTGGGGCTG3′ |
| | GadA.B1.500-3 | 50 | 5′CTTATCTACTCGAATTTGGC3′ |
| | GadA.B2.GBL-5 | 51 | 5′CTTCACGAGGCAGACCTCAGCGCCGATAACATAACGTTGTAAAAAC3′ |
| | GadA.A2.GBL-3 | 52 | 5′GATTTTGAGACACAACGTGGCTTTCATTTCGAACTCCTTAAATTTATTTG3′ |
| | GadA.ext-5 | 53 | 5′GTTGCGCGGAGATGAAAATG3′ |
| | GadA.ext-3 | 54 | 5′CATGAAGATTTAATGCCTCC3′ |
| lctR | LctR.A1.500-5 | 55 | 5′GCACTGCTCTCGATTGTCTG3′ |
| | LctR.B1.500-3 | 56 | 5′GGGCCGCTCATACCTGAATG3′ |
| | LctR.B2.GBL-5 | 57 | 5′CTTCACGAGGCAGACCTCAGCGCCTGATTATTTCCGCAGCCAGCGAT3′ |
| | LctR.A2.GBL-3 | 58 | 5′GATTTTGAGACACAACGTGGCTTTCCATTAAGGAATCATCCACGTTAAG3′ |
| | LctR.ext-5 | 59 | 5′GGTGGCGCGCTGTATGAGTG3′ |
| | LctR.ext-3 | 60 | 5′CCTAAATCATGTGGACC3′ |
| malM | MalMG.A1.500-5 | 61 | 5′ACGACTCCAGCGGATCGCGCGGCAAC3′ |
| to | MalMG.B1.500-3 | 62 | 5′CAATAGTGGAATTGTTGCTTTATC3′ |
| MalG | MalMG.B2.GBL-5 | 63 | 5′CTTCACGAGGCAGACCTCAGCGCCTAGCCCTTGTGGAGGTTCCTGCAAT3′ |
| | MalMG.A2.GBL-3 | 64 | 5′GATTTTGAGACACAACGTGGCTTTCATTTCTCATCCTTGTTTTATC3′ |
| | MalMG.ext-5 | 65 | 5′GGTTTTCGACCAGTTTGACTAAG3′ |
| | MalMG.ext-3 | 66 | 5′CGTTGGTGCTGTTAGCACTGTATC3′ |
| mdh | Mdh.A1.500-5 | 67 | 5′GCATAAGTCACCCGATATGGTGG3′ |
| | Mdh.B1.500-3 | 68 | 5′CTCGCTGGGCGAACTGATGGG3′ |
| | Mdh.B2.GBL-5 | 69 | 5′CTTCACGAGGCAGACCTCAGCGCCTAATTGATTAGCGGATAATAAAAAAC3′ |
| | Mdh.A2.GBL-3 | 70 | 5′GATTTTGAGACACAACGTGGCTTTCATCCTAAACTCCTTATTATATTG3′ |
| | Mdh.ext-5 | 71 | 5′CTGCAACGCGGCGACGATTTC3′ |
| | Mdh.ext-3 | 72 | 5′GGCAAAACTTCCTCCAAACCG3′ |
| nifS | NifS.A1.500-5 | 73 | 5′CCTTTCTTATCTGGAACAAC3′ |
| | NifS.B1.500-3 | 74 | 5′CGCCCAGACGCAGGCCAAAC3′ |
| | NifS.B2.GBL-5 | 75 | 5′CTTCACGAGGCAGACCTCAGCGCCTAATCGGTATCGGAATCAG3′ |
| | NifS.A2.GBL-3 | 76 | 5′GATTTTGAGACACAACGTGGCTTTCATTGCTCTATAAACTCCGTACATCAC3′ |
| | NifS.ext-5 | 77 | 5′CATGAGACTGACATCTAAAG3′ |
| | NifS.ext-3 | 78 | 5′CTTCTTTTACGAAGTCCAGC3′ |
| nifU | NifU.A1.500-5 | 79 | 5′CATCGCAAAAGAAGAGATGG3′ |
| | NifU.B1.500-3 | 80 | 5′CTCAGCGCCTGGGTATCGAG3′ |
| | NifU.B2.GBL-5 | 81 | 5′CTTCACGAGGCAGACCTCAGCGCCTAAGAGTTGAGGTTTGGTTATG3′ |
| | NifU.A2.GBL-3 | 82 | 5′GATTTTGAGACACAACGTGGCTTTCATTATAAATTCTCCTGATTC3′ |
| | NifU.ext-5 | 83 | 5′GGTGCGCTGTATGTACGTCG3′ |
| | NifU.ext-3 | 84 | 5′GGTTAATGGTTGCAGATTGC3′ |
| nlpE | NlpE.A1.500-5 | 85 | 5′ACATGTTGCTATTCCCGATG3′ |
| | NlpE.B1.500-3 | 86 | 5′GCAGTGTGGGCGAAGGAGAC3′ |
| | NlpE.B2.GBL-5 | 87 | 5′CACGAGGCAGACCTCAGCGCTAACCCGTCTTGAGACAGAAACAAAC3′ |
| | NlpE.A2.GBL-3 | 88 | 5′TTGAGACACAACGTGGCTTTCATCCATTCCTTCTTTTTATTCCCG3′ |
| | NlpE.ext-5 | 89 | 5′ATCTTTCCGTCTGGTATCTG3′ |
| | NlpE.ext-3 | 90 | 5′GACTCGCCAGATGTGCTCAC3′ |
| pspA to | PspAE.A1.500-5 | 91 | 5′CCCGAGCTCACCATCATCGGTGCCGTAGCGAG3′ |
| pspE | PspAE.B1.500-3 | 92 | 5′GATAATCAATTACCGAAAAGCCATC3′ |
| | PspAE.B2.GBL-5 | 93 | 5′CTTCACGAGGCAGACCTCAGCGCCTAAAAGAATTCACCATGAGCGG3′ |
| | PspAE.A2.GBL-3 | 94 | 5′GATTTTGAGACACAACGTGGCTTTCCATAATGTTGTCCTCTTGATTTCTG3′ |
| | PspAE.ext-5 | 95 | 5′CAGTTCACCGTACTCAATCACGC3′ |
| | PspAE.ext-3 | 96 | 5′CGAGTTGCTGAATATCCTGCCACTCC3′ |
| rbsB | RbsB.A1.500-5 | 97 | 5′GGTATTGGTCGTCCGCTGGG3′ |
| | RbsB.B1.500-3 | 98 | 5′CGCTCACGTTGCGCTTCCAC3′ |
| | RbsB.B2.GBL-5 | 99 | 5′CTTCACGAGGCAGACCTCAGCGCCTAGTTTTAATCAGGTTGTATG3′ |
| | RbsB.A2.GBL-3 | 100 | 5′GATTTTGAGACACAACGTGGCTTTCATATTCAAGATGTCCTGTAG3′ |
| | RbsB.ext-5 | 101 | 5′GGCGTGACCATGGTTTATAC3′ |
| | RbsB.ext-3 | 102 | 5′GAAGTTCGCGAGCCGGAGCC3′ |
| rpoE | RpoE.A1.500-5 | 103 | 5′GACCTGATGCTGGTCAGCCAGGCGTAG3′ |
| | RpoE.B1.500-3 | 104 | 5′CGCTTCAGAAGGTACTCCCAG3′ |
| | RpoE.B2.GBL-5 | 105 | 5′CTTCACGAGGCAGACCTCAGCGCCCAGGCGTTGACGATAGCGGG3′ |
| | RpoE.A2.GBL-3 | 106 | 5′GATTTTGAGACACAACGTGGCTTTCATCCGAGGTAAAGTCTCCCCA3′ |
| | RpoE.ext-5 | 107 | 5′GAACCTTCCGTTACCGGGCCTTTAC3′ |
| | RpoE.ext-3 | 108 | 5′GCAACATTGCATTAATGCGACGAC3′ |
| rseA* | RseA.A1.500-5 | 109 | 5′GCATAAAGTGGCGAGTCTGG3′ |
| | RseA.B1.500-3 | 110 | 5′GTAATTTCGATTCGGTGTCC3′ |
| | RseA.B2.frtL-5 | 111 | 5′GAACTTCGGAATAGGAACTAAGTTTGAGCAGGCGCAAACCCAGC3′ |
| | RseA.A2.frtL-3 | 112 | 5′GAAGCAGCTCCAGCCTACACCATGCCTAATACCCTTATCC3′ |
| | RseA.ext-5 | 113 | 5′GGTCCTGGTTGAACGGGTCC3′ |
| | RseA.ext-3 | 114 | 5′GTTCCAGCGTTTCACCATCG3′ |
| rseB | RseB.A1.500-5 | 115 | 5′CCATTTCGATATCTCTTCAC3′ |
| | RseB.B1.500-3 | 116 | 5′CGTCCTCGCATTTGTTATGC3′ |
| | RseB.B2.GBL-5 | 117 | 5′CTTCACGAGGCAGACCTCAGCGCCATGATCAAAGAGTGGGCTAC3′ |
| | RseB.A2.GBL-3 | 118 | 5′GATTTTGAGACACAACGTGGCTTTCATTACTGCGATTGCGTTCC3′ |
| | RseB.ext-5 | 119 | 5′CTTAATCCGTGACTCAATGC3′ |
| | RseB.ext-3 | 120 | 5′GAAATGTTCATACCGTATGG3′ |
| sixA | SixA.A1.500-5 | 121 | 5′CGCACCGCAGGTTGCTGAAC3′ |
| | SixA.B1.500-3 | 122 | 5′GTGATGTTTTCACTCCCCTGATTC3′ |
| | SixA.B2.GBL-5 | 123 | 5′CTTCACGAGGCAGACCTCAGCGCCTGATGAGTTCCAAATTATGC3′ |
| | SixA.A2.GBL-3 | 124 | 5′GATTTTGAGACACAACGTGGCTTTCATATTGCACCGCTTTTGTTAACCAG3′ |
| | SixA.ext-5 | 125 | 5′GCTGATTGGCACACAAGGGC3′ |
| | SixA.ext-3 | 126 | 5′CATTGATTCAGTCAATAGCCAATG3′ |
| sodC | SodC.A1.500-5 | 127 | 5′GCAATCACGTCTGCCGTTTACC3′ |
| | SodC.B1.500-3 | 128 | 5′GATCGGATGCTCGTAAAAGCC3′ |
| | SodC.B2.GBL-5 | 129 | 5′CTTCACGAGGCAGACCTCAGCGCCCCGATCAACCTAAACCGCTGGG3′ |
| | SodC.A2.GBL-3 | 130 | 5′GATTTTGAGACACAACGTGGCTTTCATAGGACCTCCGTTCATTG3′ |
| | SodC.ext-5 | 131 | 5′CGTTCAAACATCTGCATCAGAG3′ |
| | SodC.ext-3 | 132 | 5′GGCGTCGCGTTGGCGTGGTTAG3′ |
| spy | Spy.A1.500-5 | 133 | 5′GACACGCTGAATTTTATGCC3′ |
| | Spy.B1.500-3 | 134 | 5′CTGCCCTGCCGTCAGTTTCG3′ |
| | Spy.B2.GBL-5 | 135 | 5′CTTCACGAGGCAGACCTCAGCGCCTAATCTTTCAGCCAAAAAACTTAAGAC3′ |
| | Spy.A2.GBL-3 | 136 | 5′GATTTTGAGACACAACGTGGCTTTCCATATTCTATATCCTTCCTTTC3′ |
| | Spy.ext-5 | 137 | 5′GTCGGTATCGTGAGAACACC3′ |
| | Spy.ext-3 | 138 | 5′CTTACAGACATCCAGGCGTG3′ |
| sucA | SucA.A1.500-5 | 139 | 5′GGCTTGTTAGCGGCATATCG3′ |
| | SucA.B1.500-3 | 140 | 5′GACACGTTTTTCACTACGTG3′ |
| | SucA.B2.GBL-5 | 141 | 5′CTTCACGAGGCAGACCTCAGCGCCTAAATAAAGGATACACAATG3′ |
| | SucA.A2.GBL-3 | 142 | 5′GATTTTGAGACACAACGTGGCTTTCATCGTGATCCCTTAAGCATC3′ |
| | SucA.ext-5 | 143 | 5′CGCGAGCATTTACAGATGCC3′ |
| | SucA.ext-3 | 144 | 5′GCTTCACCGTACTGCTTACG3′ |
| sulA | SulA.A1.500-5 | 145 | 5′CAGCTTCAGTTGATTTCGCC3′ |
| | SulA.B1.500-3 | 146 | 5′CAGTTGGTTTTCATGGGTCG3′ |
| | SulA.B2.GBL-5 | 147 | 5′CTTCACGAGGCAGACCTCAGCGCCTAAGTAAATTTAGGATTAATCCTG3′ |
| | SulA.A2.GBL-3 | 148 | 5′GATTTTGAGACACAACGTGGCTTTCCATAATCAATCCAGCCCCTG3′ |
| | SulA.ext-5 | 149 | 5′GCAAATCTTTCAGTCTTTCC3′ |
| | SulA.ext-3 | 150 | 5′CATTTCAAAGCCAACATACG3′ |
| tatE | TatE.A1.500-5 | 151 | 5′GTCTGATGACCTGTTATGAC3′ |
| | TatE.B1.500-3 | 152 | 5′CAACGCCACCAGATGTGTTC3′ |
| | TatE.B2.GBL-5 | 153 | 5′CTTCACGAGGCAGACCTCAGCGCCTGACGTGGCGAGCAGGACGC3′ |
| | TatE.A2.GBL-3 | 154 | 5′GATTTTGAGACACAACGTGGCTTTCATAGATACCTTCTTGAC3′ |
| | TatE.ext-5 | 155 | 5′TGATGCTGGTAATGAAATCG3′ |
| | TatE.ext-3 | 156 | 5′CGCGGTCGTATGGATCGTGC3′ |
| ybeD | YbeD.A1.500-5 | 157 | 5′TACTTTTAAAGGCCGTGAAG3′ |
| | YbeD.B1.500-3 | 158 | 5′GCCCGAGGATGCGCTTCTAT3′ |
| | YbeD.B2.GBL-5 | 159 | 5′CTTCACGAGGCAGACCTCAGCGCCTAACTCGCTTCTCCGTTAC3′ |
| | YbeD.A2.GBL-3 | 160 | 5′GATTTTGAGACACAACGTGGCTTTCATGTCAGCTCCGGCGTAAC3′ |
| | YbeD.ext-5 | 161 | 5′CGGACACACTGACAAAGCAG3′ |
| | YbeD.ext-3 | 162 | 5′CCATATTGACGTTTAATGCC3′ |
| ybjF | YbjF.A1.500-5 | 163 | 5′TCATGGAAGACGAAACGTTG3′ |
| | YbjF.B1.500-3 | 164 | 5′CGGAAGTGAAAACTGTCTCT3′ |
| | YbjF.B2.GBL-5 | 165 | 5′CTTCACGAGGCAGACCTCAGCGCCTAAAAAAGCCGCATGTG3′ |
| | YbjF.A2.GBL-3 | 166 | 5′GATTTTGAGACACAACGTGGCTTTCATACATTGACCTTCACATC3′ |
| | YbjF.ext-5 | 167 | 5′CAACCTGGCTACATAATGCC3′ |
| | YbjF.ext-3 | 168 | 5′GATACCTACAAAACGTTTGC3′ |
| yccA | YccA.A1.500-5 | 169 | 5′CGGGCGGTGGGGATGTTTAG3′ |
| | YccA.B1.500-3 | 170 | 5′CAGTGGTTAAAGAGTGGCGG3′ |
| | YccA.B2.GBL-5 | 171 | 5′CTTCACGAGGCAGACCTCAGCGCCTAATCTCACCCGCTAACAC3′ |
| | YccA.A2.GBL-3 | 172 | 5′GATTTTGAGACACAACGTGGCTTTCATTGAGTCACTCTCTATG3′ |
| | YccA.ext-5 | 173 | 5′CTGCACTGGCGCACGTCGCC3′ |
| | YccA.ext-3 | 174 | 5′CGATGGCAGCGTGGAAGTGG3′ |
| yceP | YceP.A1.500-5 | 175 | 5′GCGAAAACTTCTCCATTGCC3′ |
| | YceP.B1.500-3 | 176 | 5′CAGCGGGCCATAATCCCTTG3′ |
| | YceP.B2.GBL-5 | 177 | 5′CTTCACGAGGCAGACCTCAGCGCCTAACATGACATGACCATCC3′ |
| | YceP.A2.GBL-3 | 178 | 5′GATTTTGAGACACAACGTGGCTTTCATCATGGCCCCCTAATTCG3′ |
| | YceP.ext-5 | 179 | 5′CCAGTATATTCAACAGGGGG3′ |
| | YceP.ext-3 | 180 | 5′CTTCGCCAGTTGGATCCAGG3′ |
| ycfJ | YcfJ.A1.500-5 | 181 | 5′CAGGCTGCACACCAGATGGC3′ |
| | YcfJ.B1.500-3 | 182 | 5′CGGAATTTACCAACAAAGAG3′ |
| | YcfJ.B2.GBL-5 | 183 | 5′CTTCACGAGGCAGACCTCAGCGCCTAACAAGGCTGTACTCTG3′ |
| | YcfJ.A2.GBL-3 | 184 | 5′GATTTTGAGACACAACGTGGCTTTCACGGGAACACCTCCTTC3′ |
| | YcfJ.ext-5 | 185 | 5′CAGACATTTACGCTATTGGC3′ |
| | YcfJ.ext-3 | 186 | 5′GGACCTCGTCGAAGCGACCG3′ |
| ycfL | YcfL.A1.500-5 | 187 | 5′GATATATACGGCAGCAAAAC3′ |
| | YcfL.B1.500-3 | 188 | 5′GGCAATGCCTATGGCTTTAC3′ |
| | YcfL.B2.GBL-5 | 189 | 5′CTTCACGAGGCAGACCTCAGCGCCTAAGGGGTGAATCTTGATG3′ |
| | YcfL.A2.GBL-3 | 190 | 5′GATTTTGAGACACAACGTGGCTTTCATCGTTACAGACCTTTATG3′ |
| | YcfL.ext-5 | 191 | 5′GCGATTATATTTAGTGTGCG3′ |
| | YcfL.ext-3 | 192 | 5′CTGACCAGATAATTTCGCCC3′ |
| ycfR | YcfR.A1.500-5 | 193 | 5′CAGCTGTGCTTCATGCTTAG3′ |
| | YcfR.B1.500-3 | 194 | 5′GCCGGCTGGACTGGATAACC3′ |
| | YcfR.B2.GBL-5 | 195 | 5′CTTCACGAGGCAGACCTCAGCGCCTAAGCATTAACCCTCATT3′ |
| | YcfR.A2.GBL-3 | 196 | 5′GATTTTGAGACACAACGTGGCTTTCATAATAGTGGCCTTATGC3′ |
| | YcfR.ext-5 | 197 | 5′CATGAAGCAGCCTGCCGGGG3′ |
| | YcfR.ext-3 | 198 | 5′GACAAACGTGCAAACCCAAC3′ |
| ydcI | YdcI.A1.500-5 | 199 | 5′GTCGAATGTACCGGCACCCC3′ |
| | YdcI.B1.500-3 | 200 | 5′CATCAACAGTATTGCTTTCC3′ |
| | YdcI.B2.GBL-5 | 201 | 5′CTTCACGAGGCAGACCTCAGCGCCTGAAAGGTGAAGGGATCTGTC3′ |
| | YdcI.A2.GBL-3 | 202 | 5′GATTTTGAGACACAACGTGGCTTTCATAAGCGATGTTAAAAAC3′ |
| | YdcI.ext-5 | 203 | 5′GCGTGTCGTATTCTTCTTGC3′ |
| | YdcI.ext-3 | 204 | 5′CGCTTCATCTCACTGAGGAC3′ |
| yebE | YebE.A1.500-5 | 205 | 5′CAAAAAATTGTCGGTCAGGC3′ |
| | YebE.B1.500-3 | 206 | 5′GCATATTCACAGCCTGGTTC3′ |
| | YebE.B2.GBL-5 | 207 | 5′CTTCACGAGGCAGACCTCAGCGCCTAATTCCGCTCTCTGGATAG3′ |
| | YebE.A2.GBL-3 | 208 | 5′GATTTTGAGACACAACGTGGCTTTCATATTTGCTCCTCAATAAC3′ |
| | YebE.ext-5 | 209 | 5′GTGAAGATCTGGATGCTGCC3′ |
| | YebE.ext-3 | 210 | 5′GGTGTTATCGGGCGTAATCG3′ |
| yfcX | YfcX.A1.500-5 | 211 | 5′CGCAAACACGGAACGGTAAC3′ |
| | YfcX.B1.500-3 | 212 | 5′GAGATCACCAGTACCGAAGC3′ |
| | YfcX.B2.GBL-5 | 213 | 5′CTTCACGAGGCAGACCTCAGCGCCTAAGAAGGTCAAAGCTATATGAA3′ |
| | YfcX.A2.GBL-3 | 214 | 5′GATTTTGAGACACAACGTGGCTTTCATTATTCCGCCTCCAGAACCA3′ |
| | YfcX.ext-5 | 215 | 5′GGTGATGACTGCCTTTATCC3′ |
| | YfcX.ext-3 | 216 | 5′CATCTTCAGATTACACGGGC3′ |
| yggN | YggN.A1.500-5 | 217 | 5′GAACCGTAGCCGTCGTCTGC3′ |
| | YggN.B1.500-3 | 218 | 5′CATCGTGTCGGTACCGTGGG3′ |
| | YggN.B2.GBL-5 | 219 | 5′CTTCACGAGGCAGACCTCAGCGCCTAATCCTCTATTTTAAGACG3′ |
| | YggN.A2.GBL-3 | 220 | 5′GATTTTGAGACACAACGTGGCTTTCATAGTCTTCCCTCAAG3′ |
| | YggN.ext-5 | 221 | 5′GTGATGTCTTCTATTGACGG3′ |
| | YggN.ext-3 | 222 | 5′GTTGGCGGAGGCTTTATCAG3′ |
| yghO | YghO.A1.500-5 | 223 | 5′CGACCAAGGTGCCTTGAGTC3′ |
| | YghO.B1.500-3 | 224 | 5′GCAGCCGCGAACGCTGTACG3′ |
| | YghO.B2.GBL-5 | 225 | 5′CTTCACGAGGCAGACCTCAGCGCCTAATACCAGCTAACTCAGGTTC3′ |
| | YghO.A2.GBL-3 | 226 | 5′GATTTTGAGACACAACGTGGCTTTATTAAGGAAGGTGCGAACAAGTC3′ |
| | YghO.ext-5 | 227 | 5′CTGCTCTTTGTTCTTGGTCG3′ |
| | YghO.ext-3 | 228 | 5′GCGCAGGGTCGCGATTCTTCG3′ |
| ygiB | YgiB.A1.500-5 | 229 | 5′GCGATGGAAGCGGGCTACTC3′ |
| | YgiB.B1.500-3 | 230 | 5′GTTCACGCAGCTCAACGAAG3′ |
| | YgiB.B2.GBL-5 | 231 | 5′CTTCACGAGGCAGACCTCAGCGCCTGATACCGATGGAAAGAGTC3′ |
| | YgiB.A2.GBL-3 | 232 | 5′GATTTTGAGACACAACGTGGCTTTTCATTTTTGTCTTCCGGGACC3′ |
| | YgiB.ext-5 | 233 | 5′GAATGGTTAACTCGCAGGTG3′ |
| | YgiB.ext-3 | 234 | 5′CCTGATCCTGTAAATCCGTG3′ |
| yhhY | YhhY.A1.500-5 | 235 | 5′CGCTGGTGAAATGGATATGG3′ |
| | YhhY.B1.500-3 | 236 | 5′GATAAAAAAGCGCCTCTTAG3′ |
| | YhhY.B2.GBL-5 | 237 | 5′CTTCACGAGGCAGACCTCAGCGCCTAAGATAGTGCCCTTTTTCTG3′ |
| | YhhY.A2.GBL-3 | 238 | 5′GATTTTGAGACACAACGTGGCTTTCATTCCTTTGTCCTCTTTGG3′ |
| | YhhY.ext-5 | 239 | 5′GTTTCGCGTACTCGAAATGG3′ |
| | YhhY.ext-3 | 240 | 5′CGATAAGATGTTGACAGAGG3′ |
| yiaH | YiaH.A1.500-5 | 241 | 5′GGAAAAAGCAGGGCTTAACG3′ |
| | YiaH.B1.500-3 | 242 | 5′GTCAAATGCGTTTGTTTCGC3′ |
| | YiaH.B2.GBL-5 | 243 | 5′CTTCACGAGGCAGACCTCAGCGCCTAAGTAAAAGCCCGGTCACATTGGAC3′ |
| | YiaH.A2.GBL-3 | 244 | 5′GATTTTGAGACACAACGTGGCTTTCATCTGTGTCTCTGTATCTG3′ |
| | YiaH.ext-5 | 245 | 5′CAAGCCCTGGAAGGTCCTGG3′ |
| | YiaH.ext-3 | 246 | 5′CATATCTGCCAGTTAGTTGC3′ |
| yjbO | YjbO.A1.500-5 | 247 | 5′CGATTAACGGTGGTATCAAG3′ |
| | YjbO.B1.500-3 | 248 | 5′CCGTGGGCAGAGACACCTGG3′ |
| | YJbO.B2.GBL-5 | 249 | 5′CTTCACGAGGCAGACCTCAGCGCCTAAGGGATTGTGCGGATGATCACAAC3′ |
| | YjbO.A2.GBL-3 | 250 | 5′GATTTTGAGACACAACGTGGCTTTCATGATGCTCTCCCAAATATG3′ |
| | YjbO.ext-5 | 251 | 5′GCAAAGGCGAGTGTGAGATG3′ |
| | YjbO.ext-3 | 252 | 5′GAGCGGTTAAAAGAGATCAC3′ |
| yneA | YneA.A1.500-5 | 253 | 5′GGCTGCATAAAACCCATGCC3′ |
| | YneA.B1.500-3 | 254 | 5′CGACTGATGTTCATATTCGC3′ |
| | YneA.B2.GBL-5 | 255 | 5′CTTCACGAGGCAGACCTCAGCGCCTGATGTGCATTACTTAACCG3′ |
| | YneA.A2.GBL-3 | 256 | 5′GATTTTGAGACACAACGTGGCTTTCATGAAGATATCCTTTATGG3′ |
| | YneA.ext-5 | 257 | 5′GCTAACCTGGATGTGCTGGG3′ |
| | YneA.ext-3 | 258 | 5′GGTACCGGACATCCGGCAAC3′ |
| yoaB | YoaB.A1.500-5 | 259 | 5′CCGGCAGATCGCCCCCCGCC3′ |
| | YoaB.B1.500-3 | 260 | 5′GGTGTTGGCGCTGATACATC3′ |
| | YoaB.B2.GBL-5 | 261 | 5′CTTCACGAGGCAGACCTCAGCGCCTAAGCTTTATCGAAGCAAAATAAG3′ |
| | YoaB.A2.GBL-3 | 262 | 5′GATTTTGAGACACAACGTGGCTTTCATCATTTTGTCCTCATTATAC3′ |
| | YoaB.ext-5 | 263 | 5′CCACGCCTGTGAATCTTCCG3′ |
| | YoaB.ext-3 | 264 | 5′CCAGGGTTCCAGCCTTCCTG3′ |
| yqcC | YqcC.A1.500-5 | 265 | 5′CTGTAAGCGCCTTGTAAGAC3′ |
| | YqcC.B1.500-3 | 266 | 5′CGAAGCTGATGTTTGCGTCC3′ |
| | YqcC.B2.GBL-5 | 267 | 5′CTTCACGAGGCAGACCTCAGCGCCTAATGCTGGAAATACTCTATC3′ |
| | YqcC.A2.GBL-3 | 268 | 5′GATTTTGAGACACAACGTGGCTTTCATAAAGCAACCTCAATAAG3′ |
| | YqcC.ext-5 | 269 | 5′CTTAAGCCTCTTCTGTAATC3′ |
| | YqcC.ext-3 | 270 | 5′GGCCCGCGTGAATAGTCAGC3′ |
| yqeC | YqeC.A1.500-5 | 271 | 5′GGGGATGCCATTATGGAGTG3′ |
| | YqeC.B1.500-3 | 272 | 5′CACCAAACGACTCAGCATGG3′ |
| | YqeC.B2.GBL-5 | 273 | 5′CTTCACGAGGCAGACCTCAGCGCCTAGCGGCCCGGGTATTCCGGG3′ |
| | YqeC.A2.GBL-3 | 274 | 5′GATTTTGAGAGACAACGTGGCTTTCACGAGTCTTTATGACCTC3′ |
| | YqeC.ext-5 | 275 | 5′CTGCATTTTCTATTTCGACG3′ |
| | YqeC.ext-3 | 276 | 5′GAACCTTGCGACGACTTGCC3′ |
| λatt-gfp | ATT.A1.500-5 | 277 | 5′CGATGGCGATAATATTTCACC3′ |
| | ATT.B1.500-3 | 278 | 5′CCCTGATACTCACCAGGCATC3′ |
| | ATT.B2.xfp-5 | 279 | 5′TGAGTAGGACAAATCCGCCGCTAAAAAAGCAGGCTTCAAC3′ |
| | ATT.A2.xfp-3 | 280 | 5′GCGTTTTTTATTGGTGAGAATTACTAACTTGAGCGAAACG3′ |
| | ATT-ext5 | 281 | 5′GGCGATAAATTGCCGCATCG3′ |
| | ATT-ext3 | 282 | 5′TGCCACCATCAAGGGAAAGCCC3′ |

^{a}Gene names according to E. coli Colibri database.
^{b}nomenclature according to Institute Pasteur database.
*Genes inactived by a removable frt kanamycin cassette.

TABLE 7

Primers used for the Q-RT-PCR experiments.
Primers were designed to amplify about 200-bp
internal gene sequence.

| Target | Primers | | |
|---|---|---|---|
| genes name | name | SEQ ID NO: | Primers sequence |
| cpxP | cpxP-RT-5 | 283 | 5′ CGCTGGCAGTCAGTTCATTAAGCC 3′ |
| | cpxP-RT-3 | 284 | 5′ GTCTCCAGTTCGCTAACATTAAC 3′ |
| cyoD | cyoD-RT-5 285 | 5′ CTACCGATCACAGCGGCGCGTCCC 3′ | |
| | cyoD-RT-3 | 286 | 5′ GTTCCAGCCTTCATCTGATTTGG 3′ |
| fimA | fimA-RT-5 | 287 | 5′ CTGGCAATCGTTGTTCTGTCGGCTC 3′ |
| | fimA-RT-3 | 288 | 5′ GCTCCTTCCTGTGCCAGCGATGCG 3′ |
| sucA | sucA-RT-5 | 289 | 5′ GAACAGCTCTATGAAGACTTCTTAAC 3′ |
| | sucA-RT-3 | 290 | 5′ GCTGCAGGACTTTAACCTGCTTCACAT 3′ |
| ycfJ | ycfJ-RT-5 | 291 | 5′ GTTGGCGGGTATCGGGATTGGTGTC 3′ |
| | ycfJ-RT-3 | 292 | 5′ [symbol] GTAATGCGATTTTCATCCTGCACC 3′ |
| ycfR | ycfR-RT-5 | 293 | 5′ CCCTCATCGCTGCGGCGATTTTAAGC 3′ |
| | ycfR-RT-3 | 294 | 5′ [symbol] CCGGTTACAGAAGTAATACGGAAAG 3′ |
| yebE | yebE-RT-5 | 295 | 5′ GGCTGCTGGTCGCAAATAAATCAG 3′ |
| | yebE-RT-3 | 296 | 5′ [symbol] GCAAGGATCAAACGTGCTGTACGC 3′ |

1. A method for identifying a substance which inhibits the formation of a bacterial biofilm, comprising
providing a host cell expressing at least one protein encoded by a gene selected from the group consisting of lctR, recA, mdh, rbsB, msrA, finA, tatE, pspF, cpxP, spy, ycfJ, ycfR, yoaB, yqcc, yggN, ymcA, yccA, yfcx, yghO, ycP, and ycuB;
contacting the host cell with the substance;
measuring the level of at least one protein or at least one RNA transcript of the at least one gene after said contacting; and
comparing the level of the at least one of the protein or RNA transcript of the at least one gene after said contacting with a host cell not contacted with the substance; wherein a reduced level of the at least one protein or the at least one RNA transcript relative to the cell not contacted with the substance indicates that the substance inhibits the formation of a bacterial biofilm.

2. The method of claim 1, wherein the bacterial biofilm is an E. coli biofilm.

3. The method of claim 1, wherein the at least one gene is yccA.

4. The method of claim 1, wherein the at least one gene is ycfJ.

5. The method of claim 1, wherein the at least one gene is yceP.

6. The method of claim 1, wherein the at least one gene is lctR.

7. The method of claim 1, wherein the at least one gene is recA.

8. The method of claim 1, wherein the at least one gene is mdh.

9. The method of claim 1, wherein the at least one gene is rbsB.

10. The method of claim 1, wherein the at least one gene is msrA.

11. The method of claim 1, wherein the at least one gene is finA.

12. The method of claim 1, wherein the at least one gene is tatE.

13. The method of claim 1, wherein the at least one gene is pspF.

14. The method of claim 1, wherein the at least one gene is cpxP.

15. The method of claim 1, wherein the at least one gene is spy.

16. The method of claim 1, wherein the at least one gene is ycfR.

17. The method of claim 1, wherein the at least one gene is yoaB.

18. The method of claim 1, wherein the at least one gene is yqcC.

19. The method of claim 1, wherein the at least one gene is yggN.

20. The method of claim 1, wherein the at least one gene is ymcA.

21. The method of claim 1, wherein the at least one gene is yfcx.

22. The method of claim 1, wherein the at least one gene is yghO.

23. The method of claim 1, wherein the at least one gene is yceP.

24. The method of claim 1, wherein the at least one gene is ycuB.

25. The method of claim 1, further comprising contacting a bacterial biofilm with the substance and measuring the inhibition of the bacterial biofilm growth relative to a bacterial biofilm not contacted with the substance.

26. A substance obtained by the method of claim 1.

27. A substance obtained by the method of claim 2.

28. A substance obtained by the method of claim 3.

29. A substance obtained by the method of claim 4.

30. A substance obtained by the method of claim 5.

31. A substance obtained by the method of claim 6.

32. A method of inhibiting the formation of a bacterial biofilm, comprising contacting the biofilm with at least one substance identified according to claim 1.

33. A method of inhibiting the formation of a bacterial biofilm on at least on substrate, comprising contacting the at least one substrate on which a biofilm is forming with at least one substance identified according to claim 1.

34. A method of treating at least one substrate on which a biofilm has developed, comprising contacting the at least one substrate with at least one substance identified according to claim 1.

35. A method of inhibiting the formation of a biofilm on at least one substrate which is susceptible to biofilm formation, comprising contacting the at least one substrate with at least one substance identified according to claim 1.

36. A method for detecting differentially expressed polynucleotide sequences which are specifically correlated with a mature bacterial biofilm, said method comprising:
obtaining a polynucleotide sample; labeling said polynucleotide sample by reacting said polynucleotide sample with a labeled probe immobilized on a solid support wherein said probe comprises at least one polynucleotide sequence selected from the group consisting of lctR, recA, mdh, rbsB, msrA, finA, tatE, pspF, cpxP, spy, ycfJ, ycfR, yoaB, yqcC, yggN, ymcA, yccA, yfcx, yghO, ycP, and ycuB or an expression product encoded by any of the polynucleotide sequences; and detecting a polynucleotide sample reaction product.

37. The method of claim 36, further comprising obtaining a control polynucleotide sample, labeling said control sample by reacting said control sample with said labeled probe, detecting a control sample reaction product, and comparing the amount of said polynucleotide sample reaction product to the amount of said control sample reaction product.

38. The method of claims 36, wherein RNA or mRNA is isolated from said polynucleotide sample.

39. The method of claim 38, wherein mRNA is isolated from said polynucleotide sample and cDNA is obtained by reverse transcription of said mRNA.

40. The method of claim 36, wherein said labeling is performed by hybridizing the polynucleotide sample with the labeled probe.

41. The method of claim 36, wherein said method is used for detecting mature bacterial biofilms.

42. The method of claim 36, wherein the bacterial biofilm is an Escherichia coli biofilm.

43. The method of claim 36, wherein the expression product is detected and is involved in a receptor-ligand interaction, and the detecting comprises detecting an interaction between a receptor and a ligand.

44. The method of claim 36, wherein the label is selected from the group consisting of radioactive, colorimetric, enzymatic, molecular amplification, bioluminescent, fluorescent labels, and mixtures thereof.

45. A method of detecting significantly overexpressed genes correlated with a mature bacterial biofilm comprising detecting at least one polynucleotide sequence or subsequence of a polynucleotide selected from the group consisting of lctR, recA, mdh, rbsB, msrA, finA, tatE, pspF, cpxP, spy, ycfJ, ycfR, yoaB, yqcC, yggN, ymcA, yccA, yfcx, yghO, ycP, and ycuB or detecting at least one product encoded by said polynucleotide library in a sample obtained from a patient.

46. A method according to claim 45, further comprising comparing an amount of said at least one polynucleotide sequence or subsequence or product encoded by said polynucleotide sequence with an amount of said polynucleotide sequence or subsequence or product encoded by said polynucleotide sequence or subsequence obtained from a control sample.

47. The method according to claim 45, comprising extracting mRNA from said polynucleotide sample.

48. The method according to claim 47, comprising reverse transcribing said mRNA to cDNA.

49. The method according to claim 45, comprising hybridizing said at least one polynucleotide sequence or subsequence with mRNA or cDNA from the polynucleotide sample.

50. The method according to claim 45, wherein the expression product is detected and is involved in a receptor-ligand interaction, and the detecting comprises detecting an interaction between a receptor and a ligand.

51. A polynucleotide library useful in the molecular characterization of a mature bacterial biofilm, said library comprising a pool of polynucleotide sequences or subsequences thereof wherein said sequences or subsequences are overexpressed in mature bacterial biofilms, further wherein said sequences or subsequences correspond substantially to one or more polynucleotide sequences selected from the group consisting of rne, lctR, dinI, glpQ, mdh, sixA, lamB, rbsB, gadA, pspA, pspB, pspC, pspD, tatE, cpxP, rseA, rpoE, spy, yebE, yqcC, yfcX, yjbO, yceP, and ygiB.

52. The polynucleotide library of claim 51, wherein the library further comprises one or more polynucleotide sequences or subsequences thereof selected from the group consisting of recA, msrA, fimA, pspF, ycfJ, ycfR, yoaB, yggN, yneA, yccA, and yghO.

53. The polynucleotide library of claim 51, wherein the library further comprises one or more polynucleotide sequences or subsequences selected from the group consisting of RplY, recA, cyoD, sucA, fdhF, cyoC, nifU, sucD, sfsA, nifS, fadB, ucpA, ftsL, sulA, eco, msrA, pspD, fimA, fimI, pspE, pspF, cutC, sodC, rseB, ycfJ, ycfR, yoaB, yhhY, yggN, yneA, ybeD, ydcI, yddL, yccA, yrdD, ybjF, yihN, 1228, ycfL, yiaH, and yqeC.

54. The polynucleotide library of claim 51, wherein the library further comprises one or more polynucleotide sequences or subsequences thereof selected from the group consisting of lysU, miaA, rluC, rplY, crl, cspD, dniR, fruR, idnR, lacI, nac, rnk, rpoS, ttk, b0299, dinG, dinP, exo, intA, recA, recN, sbmC, xthA, aceA, aceB, aldA, atpA, cyoA, cyoC, cyoD, dctA, fdhF, fdoG, glpD, glpK, nifU, pckA, sdhB, sdhD, sucA, sucB, sucD, xdhD, agp, gcd, glgS, glpX, malE, malF, malS, mglA, mglB, mrsA, pgm, rbsC, rbsD, sfsA, ansB, argC, argR, idnD, leuD, metH, nifS, putP, metK, pnuC, ubiE, fabA, fadB, fadE, fadL, pgpA, pssA, uppS, idnO, ucpA, ftsL, sulA, dnaJ, dnaK, eco, fkpA, glnE, htpG, htpX, msrA, amiB, ddg, fhiA, fimA, fimI, htrL, lepB, mraW, nlpB, nlpC, ompC, ompG, pspE, pspF, chaA, chaC, cutC, cysP, cysU, fur, modA, modB, modC, modE, sodC, trkH, rseB, ycfJ, ycfR, yoaB, yhhY, yggN, yneA, ybeD, ydcI, yddL, yccA, yrdD, ybjF, yihN, ycfT, yeeF, yfiE, yeeD, yliH, yfcM, ybiX, yfhF/nifA, ygfQ, ybhR, ybdH, yihR, ydcT, ygiS, ybaZ, ydaM, tfaR, yceL, yheT, yjdC, ybiW, ybiF, ynaI, yceE, yhdP, ygiE, csiE, yfdE, yeeE, yegQ, glcA, yfdW, yfeT, ygjK, ydeW, b1228, ycfL, yghO, yiaH, yqeC, ycfT, yhjJ, yceB, ybiX, ygiQ, yagV, yoeA, ybhQ, ybcI, ybbF, ybgI, yncH, yfbM, yjiM, yjfO, ychN, ynaC, ymfE, yfcN, yrbC, yfdQ, yfeY, ygiM, yhgA, yhjQ, yfcF, yfcI, yjiD, yfbP, yphB, yfbN, ylbH, ybhM, yrbL, yjfY, ynfA, yajI, yedi, yafZ, yjjU, yfhH, yafN, yrbE, yfgC, yfjQ, ycaK, yfeS, b4250, ybgA, yeeA, ypfI, b2394, yegK, ybcJ, yhiN, ypfG, ydiY, yjjJ, ycaP, and yfgJ.

55. The polynucleotide library of claim 51, wherein said biofilm are an Escherichia coli biofilm.

56. The polynucleotide library of 51, wherein said one or more polynucleotide sequences or subsequences of said pool are immobilized on a solid support to form a polynucleotide array.

57. The polynucleotide library of claim 56, wherein the solid support is selected from the group consisting of a nylon membrane, glass slide, glass beads, and a silicon chip.

58. A polynucleotide array useful to detect a mature bacterial biofilm comprising an immobilized polynucleotide library according to claim 51.
